(19) United States
(12) Patent Application Publication
(10) Pub. No.: US 2015/0231227 A1
(43) Pub. Date: Aug. 20, 2015

(54) COMPOSITIONS AND METHODS FOR GENERATING AN IMMUNE RESPONSE

(71) Applicants: Harriet L. Robinson, Atlanta, GA (US); James M. Smith, Cumming, GA (US); Jian Hua, Dunwoody, GA (US); Bernard Moss, Bethesda, MA (US); Rama R. Amara, Atlanta, GA (US); Linda S. Wyatt, Rockville, MD (US); Patricia L. Earl, Chevy Chase, MA (US); Ted M. Ross, Aspinall, PA (US); Rick A. Bright, Washington, DC (US); Salvatore T. Butera, Atlanta, GA (US); Dennis L. Ellenberger, Norcross, GA (US); Thomas M. Folks, Snellville, GA (US)

(72) Inventors: Harriet L. Robinson, Atlanta, GA (US); James M. Smith, Cumming, GA (US); Jian Hua, Dunwoody, GA (US); Bernard Moss, Bethesda, MA (US); Rama R. Amara, Atlanta, GA (US); Linda S. Wyatt, Rockville, MD (US); Patricia L. Earl, Chevy Chase, MA (US); Ted M. Ross, Aspinall, PA (US); Rick A. Bright, Washington, DC (US); Salvatore T. Butera, Atlanta, GA (US); Dennis L. Ellenberger, Norcross, GA (US); Thomas M. Folks, Snellville, GA (US)

(73) Assignee: EMORY UNIVERSITY

(21) Appl. No.: 12/749,164

(22) Filed: Mar. 29, 2010

Related U.S. Application Data
(63) Continuation of application No. 12/487,379, filed on Jun. 18, 2009.
Application No. 12/487,379 is a continuation of application No. 12/250,851, filed on Oct. 14, 2008, now abandoned.
Application No. 12/250,851 is a continuation of application No. 12/033,300, filed on Feb. 19, 2008, now abandoned.
Application No. 12/033,300 is a continuation of application No. 11/764,766, filed on Jun. 18, 2007, now abandoned.
Application No. 11/764,766 is a continuation-in-part of application No. 09/798,675, filed on Mar. 2, 2001, now abandoned.
(60) Provisional application No. 60/251,083, filed on Dec. 1, 2000.
(60) Provisional application No. 60/186,364, filed on Mar. 2, 2000.

(51) Int. Cl. A61K 39/21; A61K 39/145; C12N 7/00; A61K 39/165

(52) U.S. Cl. CPC A61K 39/21; A61K 39/165; A61K 39/145; C12N 7/00; A61K 2039/70

(57) ABSTRACT

Novel plasmid constructs useful for the delivery of DNA vaccines are provided having a transcription cassette capable of directing the expression of a vaccine nucleic acid insert encoding immunogens derived from any pathogen, including fungi, bacteria and viruses. The constructs are useful for inducing in a patient an immune response against pathogenic viruses such as HIV, measles or influenza. Immunodeficiency virus vaccine inserts of the present invention express non-infectious HIV virus-like particles (VLP) bearing multiple viral epitopes. VLPs allow presentation of the epitopes to multiple histocompatability types. Also described are methods for immunizing a patient by delivery of a novel plasmid of the present invention to a patient. Optionally, the immunization protocol may include a booster vaccination that may be a live vector vaccine such as a recombinant pox virus or modified vaccinia Arbora vector having a transcription cassette expressing the same vaccine insert as the primary immunizing vector.

This application claims the benefit of U.S. Ser. No. 60/324,845, filed Sep. 26, 2001, which is incorporated here by reference in its entirety. This application is a continuation-in-part of U.S. Ser. No. 09/798,675, filed Mar. 2, 2001, which claims the benefit of the filing dates of U.S. Ser. No. 60/251,083, filed Dec. 1, 2000, and U.S. Ser. No. 60/186,364, filed Mar. 2, 2000. The contents of U.S. Ser. Nos. 09/798,675, 60/251,083, and 60/186,364 are also incorporated here by reference in their entirety.

### GOVERNMENT SUPPORT

The work described herein may have been supported, at least in part, by grants from the National Institutes of Health (5 POI AI43045) and National Institutes of Health/National Institute of Allergy and Infectious Diseases (R21 AI44325-01). The United States Government may therefore have certain rights in this invention.

### FIELD OF THE INVENTION

The present invention is directed generally to the fields of molecular genetics and immunology. More particularly, the present invention features expression vectors (e.g., vectors comprising DNA encoding one or more antigens), and methods of immunizing animals (including humans) by administering one or more of these vectors.

### BACKGROUND OF THE INVENTION

Vaccines have had profound and long lasting effects on world health. Small poxhas been eradicated, polio is near elimination, and diseases such as diphtheria, measles, mumps, pertussis, and tetanus are contained. Nonetheless, microbes remain major killers with current vaccines addressing only a handful of the infections of man and his domesticated animals. Common infectious diseases for which there are no vaccines cost the United States $120 billion dollars per year (Robinson et al., American Academy of Microbiology, May 31-Jun. 2, 1996). In first world countries, emerging infections such as immunodeficiency viruses, as well as reemerging diseases like drug resistant forms of tuberculosis, pose new threats and challenges for vaccine development. The need for both new and improved vaccines is even more pronounced in third world countries where effective vaccines are often unavailable or cost-prohibitive. Recently, direct injections of antigen-expressing DNAs have been shown to initiate protective immune responses.

DNA-based vaccines use bacterial plasmids to express protein immunogens in vaccinated hosts. Recombinant DNA technology is used to clone cDNAs encoding immunogens of interest into euk:aryotic expression plasmids. Vaccine plasmids are then amplified in bacteria, purified, and directly inoculated into the hosts being vaccinated. DNA typically is inoculated by a needle injection of DNA in saline, or by a gene gun device that delivers DNA-coated gold beads into skin. The plasmid DNA is taken up by host cells, the vaccine protein is expressed, processed and presented in the context of self major histocompatibility (MHC) class I and class II molecules, and an immune response against the DNA-encoded immunogen is generated.

The historical foundations for DNA vaccines (also known as “genetic immunization”) emerged concurrently from studies on gene therapy and studies using retroviral vectors. Classic references for DNA vaccines include the first demonstration of the raising of an immune response (Tang et al., Nature 356: 152-154, 1992); the first demonstration of cytotoxic T cell (Tc)-mediated immunity (Ulmer et al., Science 259:1745-1749, 1993); the first demonstration of the protective efficacy of intradermal, intramuscular, intravenous, intranasal, and gene gun (or biolistic) immunizations (Fynan et al., Proc. Natl. Acad. Sci. USA 90:11478-11482, 1993; Robinson et al., Vaccine 11:957-960, 1993); the first use of genetic adjuvants (Xiang et al., Immunity 2:129-135, 1995); the first use of library immunizations (Barry et al., Nature, 377:632-635, 1995); and the first demonstration of the ability to modulate the T-helper type of an immuneresponse by the method of DNA delivery (Feltquate et al., J. Immunol. 158:2278-2284, 1997). Useful compilations of DNA vaccine information can also be found on the worldwide web.

Gene therapy studies on DNA delivery into muscle revealed that pure DNA was as effective as liposome-encapsulated DNA at mediating transfection of skeletal muscle cells (Wolff et al., Science 247:1465-1468, 1990). This unencapsulated DNA was termed “naked DNA,” a fanciful term that has become popular for the description of the pure DNA used for nucleic acid vaccinations. Gene guns, which had been developed to deliver DNA into plant cells, were also used in gene therapy studies to deliver DNA into skin. In a series of experiments testing the ability of plasmid-expressed human growth hormone to alter the growth of mice, it was realized that the plasmid inoculations, which had failed to alter growth, had elicited antibody ((Tang et al., Nature 356:152-154, 1992). This was the first demonstration of the raising of an immune response by an inoculated plasmid DNA. At the same time, with experiments using retroviral vectors, investigators demonstrated protective immune responses raised by very few infected cells (on the order of 104-105). Direct tests of the plasmid DNA that had been used to produce infectious forms of the retroviral vector for vaccination, performed in an influenza model in chickens, resulted in protective immunizations (Robinson et al., Vaccine 11:957-960, 1993).

The prevalence of HIV-1 infection has made vaccine development for this recently emergent agent a high priority for world health. Pre-clinical trials on DNA vaccines have demonstrated that DNA alone can protect against highly attenuated HIV-1 challenges in chimpanzees (Boyer et al., Nature Med. J.: 526-532, 1997), but not against more virulent SIV challenges in macaques (Lu et al., Vaccine 12.:920-923, 1997). A combination of DNA priming plus an envelope glycoprotein boost has raised neutralizing antibody-associated protection against a homologous challenge with a non-pathogenic chimera between SIV and HIV (SHIV-IIIB) (Letvin et al., Proc. Natl. Acad. Sci. USA 94:9378-9383, 1997). More recently, a comparative trial testing eight different protocols for the ability to protect against a series of challenges with SHIV s (chimeras betweensimian and human immunodeficiency viruses) revealed the best containment of challenge infections by an immunization protocol that included priming by intradermal inoculation of DNA and boosting with recombinant fowl pox virus vectors (Robinson et al., NatureMed. 5:526, 1999). This containment of challenge infections was independent of the presence of neutralizing antibody to the challenge virus. Protocols that proved less effective at containing challenge infections included immunization by both priming and boosting by intradermal or gene gun-administered DNA; immunization by priming with intradermal or gene gun-administered DNA inoculation and then boosting with a protein subunit; immunization by priming with gene gun-administered DNA inoculations and boosting with recombinant fowl pox virus; immunization with protein only; and immunization with recombinant fowl pox virus only (Robinson et al., Nature Med.:526, 1999). Early clinical trials of DNA vaccines in humans have revealed no adverse effects (MacGregor et al., Intl. Conj AIDS, 11:23, Abstract No. We.B.293, 1996) and the raising of cytolytic T cells (Calarota et al., Lancet 351:1320-1325, 1998). A number of investigators have examined the ability of co-transfected lymphokines and co-stimulatory molecules to increase the efficiency of immunization (Robinson and Pertmer, Adv. Virus Res. 55:1-74, 2000).

Of course, DNA vaccines are limited in that they can only be used to immunize patients with products encoded by DNA (e.g., proteins), and it is possible that bacterial and parasitic proteins may be atypically processed by eukaryotic cells. Another significant problem with existing DNA vaccines is the instability of some vaccine insert sequences during the growth and amplification of DNA vaccine plasmids in bacteria. Instability can arise during plasmid growth where the secondary structure of the vaccine insert or of the plasmid vector (the “backbone”) can be altered by bacterial endonucleases.

### SUMMARY OF THE INVENTION

There is a pressing need for effective vaccines, particularly against pathogens such as the human immunodeficiency (HIV) virus, which frequently mutates, and poxviruses, such as the variola virus that causes smallpox, for which there is no specific therapy. Insofar as these vaccines may be administered by DNA expression vectors and/or viruses constructed with such vectors, there is a need for plasmids that are more stable in bacterial hosts and safer in animals. Such vaccines and vectors are disclosed herein, together with methods for administering them to animals, including humans.

The present invention provides plasmid constructs that can be used to deliver a nucleic acid (e.g., DNA that encodes one or more antigens from one or more pathogens) to cells (the nucleic acids are as conventionally known, i.e., they can be any linear array of naturally occurring or synthetic nucleotides or nucleosides derived from cDNA (or mRNA) or genomic DNA, or derivatives thereof). The plasmid constructs can include, as a vaccine insert, a transcription unit (e.g., a DNA transcription unit) of a virus, bacterium, parasite or fungus or any fragments or derivatives thereof that elicit an immune response against the pathogen from which the insert was derived or obtained (the plasmid constructs may be referred to as, inter alia, expression vectors, expression constructs or, simply, plasmids, regardless of whether or not they include an insert). As described further below, therapeutically effective amounts of the plasmids of the present invention can be administered to patients. Accordingly, the invention features methods of immunizing a patient (or of eliciting an immune response in a patient, which can includemulti-epitope CD8+ T cell responses)) by administering a plasmid construct comprising a vaccine insert. The plasmid can be administered alone (i. e., a plasmid can be administered on one or several occasions without an alternative type of vaccine formulation (e.g., without administration of protein or another type of vector, such as a viral vector) and, optionally, with an adjuvant) or in conjunction with (e.g., prior to) an alternative booster immunization (e.g., a live-vectored vaccine such as a recombinant modified vaccinia Ankara vector (MV A, e.g., MV A48) comprising the same vaccine insert(s) or at least one of the same inserts as the plasmid administered as the “prime”portion of the inoculation protocol). Similarly, as described further below, one can immunize a patient (or elicit an immune response, which can include multi-epitope CDS+ T cell responses) by administering a live-vectored vaccine (e.g., MVA, including MVA48) without administering a plasmid-based (or “DNA”) vaccine. The alternative embodiments of an “MVA only” or “MVA-MVA” vaccine regimen are the same as those described herein for “DNA-MVA” regimens. For example, in either case, one can include an adjuvant and administer a variety of antigens, including those obtained from any HIV clade (e.g., clade B or clade AG).

As implied by the term “immunization” (and variants thereof), the compositions of the invention can be administered to a subject who has not yet become infected with a pathogen, but the invention is not so limited; the compositions described herein can also be administered to treat a patient who has already been exposed to, or who is known to be infected with, a pathogen (e.g., an HIV).

An advantage of DNA-based immunizations is that the immunogen can be presented by both MHC class I and class II molecules. Endogenously synthesized proteins readily enter processing pathways that load peptide epitopes onto MHC I as well as MHC II molecules. MHC I-presented epitopes raise cytotoxic T cell (Tc) responses, whereas MHC II presented epitopes raise helper T cells (Th). By contrast, immunogens that are not synthesized in cells are largely restricted to the loading of MHC II epitopes and therefore raise Th but not Tc. In addition, DNA plasmids are not infectious agents, and they can be used to focus the immune response on only those antigens desired for immunization. Another possible advantage of a DNA-based vaccine (whether used alone or in concert with a live-vectored vaccine) is that it can be manipulated to raise type 1 or type 2 T cell help. This allows the vaccine to be tailored for the type of immune response that will be mobilized to combat an infection.

The antigens encoded by DNA are necessarily proteinaceous. The terms “protein,” “polypeptide,” and “peptide” are generally interchangeable, although the term“peptide” is commonly used to refer to a short sequence of amino acid residues or a fragment of a larger protein. In any event, serial arrays of amino acid residues, linked through peptide bonds, can be obtained by using recombinant techniques (e.g., as was done for the vaccine inserts described and exemplified herein), purified from a natural source, or synthesized. Moreover, one or more amino acid residues within an antigen can be chemically modified or linked to a label, such as a fluorophore or radioisotope.

Other advantages of DNA-based vaccines (and of viral vectors, such as poxvirus-based vectors) are described below. The details of one or more embodiments of the invention are set forth in the accompanying drawings and the description below. Other features, objects, and advantages of the invention will be apparent from the description and drawings, and from the claims.

### BRIEF DESCRIPTION OF THE DRAWINGS

FIG. 1 is a schematic illustration of a plasmid construct termed pGA1. The identities and positions of elements in the vector (e.g., the promoter (here, a CMV promoter), the multi-cloning site, a terminator sequence (here, the lambda T0 terminator), and a marker gene (here, the kanamycin resistance gene)) are shown. Unique restriction endonuclease sites, which are useful for cloning vaccine inserts into the plasmid, are shown in italic type.

FIG. 2 is an illustration of the nucleotide sequence of pGA1 (SEQ ID NO:1). The boundaries of various elements in the plasmid (e.g., the CMV promoter), intron A, the tpa leader, the polyadenylation signal, etc. are indicated below the nucleotide sequence.

FIG. 3 is a schematic illustration of a plasmid construct termed pGA2. The identities and positions of elements in the vector (the promoter etc.) are shown. Unique restriction endonuclease sites, which are useful for cloning vaccine inserts into the plasmid, are shown in italic type.

FIG. 4 is an illustration of the nucleotide sequence of pGA2 (SEQ ID N0:2). The boundaries of various elements in the plasmid (e.g., the CMV promoter), the tpa leader, the polyadenylation signal, etc. are indicated below the nucleotide sequence.

FIG. 5 is a schematic illustration of a plasmid construct termed pGA3. The identities and positions of elements in the vector (the promoter etc.) are shown. Unique restriction endonuclease sites, which are useful for cloning vaccine inserts into the plasmid, are shown in italic type.

FIG. 6 is an illustration of the nucleotide sequence of pGA3 (SEQ ID NO: 3). The boundaries of various elements in the plasmid (e.g., the CMV promoter), intron A, the tpa leader, the polyadenylation signal, etc. are indicated below the nucleotide sequence.

FIGS. 7A and 7B are line graphs illustrating the levels of anti-HA IgG raised by the influenza H1 hemagglutinin expressed in the pGA3 vector (pGA3/H1) and in the pJW4303 research vector (JW4303/H1) when BALB/c mice were immunized and boosted with a low dose (0.1 μg; FIG. 7 A) or a high dose (1.0 μg; FIG. 7B) of the indicated plasmids using gene gun inoculations. A priming immunization was followed by a booster immunization at 4 weeks. The results obtained with vector only are also shown.

FIGS. 8A-8C are schematic representations of a provirus and two vaccine inserts. FIG. 8A illustrates the parent wt HIV-1-BH1O provirus from which constructs producing non-infectious virus like particles (VLPs) were produced. Sequences that were deleted in the VLP constructs are dotted. The positions and designations assigned to various regions of the HN-1-BH10 provirus are indicated in the rectangular boxes. The U3, R, and US regions that encode the long terminal repeats contain transcriptional control elements. All other indicated regions encode proteins. For clarity, products expressed by pol (Prt, RT, Int) and env (SU and TM) are indicated. FIG. 8B illustrates the JS2 vaccine insert. This 6.7 kb vaccine insert expresses the Gag, Prt, and RT sequences of the BH10 strain of HIV-1-IIIB, Tat and Vpu proteins from HIV-1-ADA, and Rev and Env proteins that are chimeras of HIV-1-ADA and HIV-1-BH10 sequences. The Gag sequences include mutations of the zinc fingers to limit packaging of viral RNA. The RT sequences encompass three point mutations to eliminate reverse transcriptase activity. Designations are the same as in FIG. 8A. The bracketed area indicates the region of HIV-1-BH10 in which sequences from HIV-1-ADA have been substituted for the HIV-1-BH1O sequences to introduce a CCR-5 using Env. The x's indicate safety mutations. FIG. 8C illustrates the JS5 insert. JS5 is a vaccine insert of approximately 6 kb that expresses Gag, Prt, RT, Vpu Tat, and Rev. JS5 is comprised of the same sequences as JS2 except that sequences in Env have been deleted. Designations are the same as in FIGS. 8A and 8B. The Rev responsive element (RRE) in the 3′ region of Env is retained in the construct.

FIGS. 9A and 9B are bar graphs illustrating Gag expression (FIG. 9A) and Env expression (FIG. 9B) from intermediates in the construction of the JS2 vaccine insert. Data were obtained following transient transfection of 293T cells. pGA1/JS 1(ADA VLP) produced higher levels of Gag and Env than did wild type HIV-1-ADA (ADA wt).

FIG. 10 is a bar graph illustrating the expression of p24 capsid in cells transiently transfected with pGA1 expressing inserts without safety mutations (pGA1/JS 1 and pGA1/JS4), inserts with point mutations in the zinc fingers and in RT (pGA1/JS2 and pGA1/JS5), and point mutations in the zinc fingers, RT, and protease (pGA1/JS3 and pGA1/JS6). Constructs expressing inserts with safety mutations in the zinc fingers and RT supported active VLP expression whereas the safety mutation in Prt did not. JS2 and JS5 were chosen for continued development based on their high levels of expression in the presence of safety mutations.

FIGS. 11A and 11B are bar graphs showing Gag expression (FIG. 11A) and Env expression (FIG. 11B) from vaccine inserts with the CMV intron A (pGA1) or without the CMV intron A (pGA2).

FIGS. 12A-12D are reproductions of Western blots of cell lysates and tissue culture supematants from 293T cells that were mock transfected (lanes labeled “1”) or transfected with pGA2/JS2 (lanes labeled “2”) or pGA1/JS5 (lanes labeled “3”), where the primary antibody was pooled from anti-HIV 1 g from infected patients (FIG. 12A), anti-p24 (FIG. 12B), anti-gp120 (FIG. 12C) and anti-RT (FIG. 12D).

FIG. 13 is a schematic representation of the parent SHIV-89.6 virus (simian-human immunodeficiency chimera) wherein the gag-pol sequences are from SIV239, and the tat, rev and env sequences are from HIV-1-89.6; pGA2/89.6 construct, and thepGA1/Gag-Pol construct.

FIG. 14 is a bar graph illustrating Gag expression from constructs pGA2/89.6; pGA1/Gag-Pol; and pGA2/JS2 in cell lysates, supernatants and in total. FIG. 15A is a schematic representation of Gag-specific CDS+ T cell responses raised over time by DNA priming and rMVA boosting, and shows Gag-CM9-tetramer data generated in high-dose intradermally DNA-immunized animals.

FIG. 15B is a schematic representation of temporal:frequencies of Gag-CM9-Mamu-A*01 tetramer-specific T cells in Mamu-A*01 vaccinated and control macaques at various times before challenge and at two weeks after challenge. The number at the upper right corner of each plot represents the frequency of tetramer-specific CD8+ T cells as a % of total CD8+ T cells. The numbers above each column of plots designate individual animals. FIG. 15C is a schematic representation of Gag-specific IFN-γ ELISPOTs inA*OJ (solid bars) and non-A*01 (hatched bars) vaccinated and non-vaccinated macaques at various times before challenge and at two weeks after challenge. Three pools of approximately 10-13 Gag peptides (22-mers overlapping by 12) were used for the analyses. The numbers above data bars represent the arithmetic mean±the standard deviation for the ELISPOTs within each group. The numbers at the top of the graphs designate individual animals. *,data not available;#, <20 ELISPOTs per 1×10⁶ peripheral blood mononucleocytes (PBMC).

FIG. 16A is a schematic representation of the height and breadth of IFN-γ producing ELISPOTs against Gag and Env in the DNA/MVA memory response. Responses against individual Gag and Env peptide pools are shown. Data for animals within a group are designated by the same symbol. FIG. 16B is a table showing the averages of the height and breadth of ELISPOT responses for the different groups. The heights are the mean±the standard deviation for the sums of the Gag and Env ELISPOTs for animals in each group. The breadths are the mean±the standard deviation for the number of Gag and Env pools recognized byanimals in each group. ELISPOT responses were determined in PBMC, during the memory phase, at 25 weeks after the rMVA booster (four weeks prior to challenge) using seven pools of Gag peptides. (approximately seven 22-mers overlapping by 12) representing about seven amino acids of Gag sequence, and 21 pools of Env peptides (approximately ten 15-mers overlapping by 11) representing about 40 amino acids of Env sequence.

FIG. 17 is a representation of the DNA sequence of a pGA2 construct comprising a pathogen vaccine insert capable of expressing the JS2 clade B HIV-1 VLP (SEQ ID NO: 4), and the protein sequences encoded thereby (SEQ ID NOs: ______).

FIG. 18 is a representation of the DNA sequence of a pGA1 construct comprising the pathogen vaccine insert capable of expressing the JS5 clade B HIV-1 Gag-pol insert (SEQ ID NO: 5), and the protein sequences encoded thereby (SEQ ID NOs: ______).

FIGS. 19A-19E are graphs. FIG. 19A shows the temporal geometric mean viral loads after challenge of vaccinated and control animals; FIG. 19B shows the geometric mean CD4 counts for vaccine-treated and control group animals at various weeks post challenge (see the legend inset in FIG. 19B); FIG. 19C is survival curve for vaccinated (dashed line) and non-vaccinated (solid line) animals. The dashed line represents all 24 vaccinated animals: FIG. 19D shows temporal viral loads for individual animals in the vaccine and control groups after challenge of vaccinated and control animals; and FIG. 19E shows temporal CD4 counts for individual animals in the vaccine and control groups after challenge of vaccinated and control animals. The key to animal numbers is given in FIG. 19E. Assays for the first 12 weeks post challenge had a background of 1000 copies of RNA per ml of plasma. Animals with loads below 1000 were scored with a load of 500. For weeks 16 and 20, the background for detection was 300 copies of RNA/ml. Animals with levels of virus below 300 were scored at 300.

FIG. 20A is a series of line graphs illustrating temporal tetramer-positive cells and viral loads in post-challenge T cell responses in vaccine and control groups.

FIG. 20B is a schematic representation of the results of intracellular cytokine assays for IFN-γ production in response to stimulation with the Gag-CM9 peptide at two weeks post-challenge, allowing evaluation of the functional status of the peak post-challenge tetramer-positive cells displayed in FIG. 15A.

FIG. 20C is a graph illustrating the results of proliferation assays at 12 weeks post-challenge.Gag-Pol-Env (solid bars) and Gag-Pol (hatched bars) produced by transient transfections were used for stimulation, and supernatants from mock-transfected cultures served as the control antigen. Proteins were used at approximately 1 μg per ml of p27Gag for stimulations. Stimulation indices are defined as the growth of cultures in the presence of viral antigens divided by the growth of cultures in the presence of mock antigen.

FIGS. 21A-21C are histomicrographs of lymph nodes. FIG. 21A shows a typical lymph node from a vaccinated macaque. There is evidence of follicular hyperplasia, which is characterized by the presence of numerous secondary follicles with expanded germinal centers and discrete dark and light zones. FIG. 21B shows a typical lymph node from an infected control animal at 12 weeks post-challenge. Follicular depletion and paracortical lymphocellular atrophy are evident. FIG. 21C shows a representative lymph node from an age-matched, uninfected macaque 12 weeks post-challenge. This tissue displays non-reactive germinal centers. FIG. 21D is a bar graph displaying the percentages of the total lymph node area occupied by germinal centers, giving a non-specific indicator of follicular hyperplasia. Uninfected controls were four age-matched rhesus macaques. FIG. 21E is a bar graph illustrating lymph node virus burden (determined by in situ hybridization using an antisense riboprobe cocktail that was complementary to SHIV-89 0.6 gag and poly. All of the examined nodes were inguinal lymph nodes.

FIGS. 22A-22D are graphs showing temporal antibody responses following challenge. Micrograms of total anti-Gag (FIG. 22A) or anti-Env (FIG. 22B) antibody were determined using ELISAs. The titers of neutralizing antibody against SHIV-89.6 (FIG. 22C) and SHIV-89.6P (FIG. 22D) were determined by MT-2 cell killing and neutral red staining Titers are the reciprocal of the serum dilution giving 50% neutralization of the indicated viruses grown in human PBMC. Symbols for animals are given in FIG. 19.

FIG. 23A shows the inverse correlation between peak vaccine raised Gag-specific IFN-γ ELISPOTs and viral loads at 2 weeks post-challenge.

FIG. 23B shows the inverse correlation between peak vaccine raised Gag-specific IFN-γ ELISPOTs and viral loads at 3 weeks post-challenge. FIG. 23C shows the dose response curves for the average height of Gag-specific IFN-γ ELISPOTS at the peak DNA-MVA response (data from FIG. 15C). FIG. 23D shows the dose response curves for the breadth of the DNA/MVA memory ELISPOT response (data from FIG. 16B). FIG. 23E shows the dose response curves for the peak anti-Gag antibody response post the MVA booster (data from FIG. 22A). The different doses of DNA raised different levels of ELISPOT and antibody responses {P<0.05). The route of DNA inoculation had a significant effect on the antibody (P=0.02), but not the ELISPOT response.

FIG. 24 shows anti-HA IgG raised by gene gun inoculation of DNAs expressing influenza hemagglutinin (HA) proteins. Mice were immunized with different doses of vaccine plasmid. Half of the mice were primed at day 0 and boosted at week 4 (A, B) and half were given a single vaccination at day 0 (C, D). A ratio of the dose of DNA to specific IgG concentrations was obtained at week 14 (E, F). Sera were obtained from mice with vector (filled squares), sHA (open circles) or sHA-3 Cd (filled circles).

FIG. 25 shows the avidity of the anti-HA IgG raised by the three different HA DNA vaccines. Sera were analyzed from week 8 (A, B) and week 14 (C, D) in an A/PR/8/34 (H1N1)-specific NaSCN-displacement ELISA. Sera were obtained from miceinoculated. Sera were obtained from mice with sHA (open circles), tmHA (open squares) or sHA-3C3d (filled circles).

FIG. 26 shows protection from weight loss after virus challenge. At week 8 (A, B) or week 14 (C, F) mice were challenged intranasally with a lethal dose of influenza virus, A/PR/8/34 (H1N1), and monitored daily for weight loss. The data are plotted as the percentage of the average initial weight. (A, C): Mice were primed and boosted with a1 μg dose of DNA vaccine. (B, D): Mice were primed and boosted with a 0.1 μg dose of DNA vaccine. (E): Mice were given a single 1 μg dose of DNA vaccine. (F): Mice were given a single 0.1 μg dose of DNA vaccine. Sera were obtained from mice with vector (filled squares), sHA (open circles), tmHA (open squares), sHA-3C3d (filled squares), naYve-mock (open triangles) or naYve-virus (filled triangles). The open cross indicates the time point at which all five mice in a group succumbed to disease.

FIG. 27 illustrates the constructs used to determine the importance of including Env in the vaccine.

FIG. 28A shows the geometric mean viral load after immunizing with Gag-Pol DNA or Gag-Pol-Env. FIG. 28B shows the geometric mean of CD4 cell loads in animals immunized withGag-Pol DNA or Gag-Pol-Env. FIG. 28C shows the viral load after immunizing with Gag-Pol DNA or Gag-Pol-Env. FIG. 28D shows the CD4 cell load after immunizing with Gag-Pol DNA or Gag-Pol-Env.

FIGS. 29A and 29B are graphs illustrating temporal frequencies of Gag-specific T cell responses in MVA-only and DNA/MVA-vaccinated animals (FIG. 29A; symbols for individual animals are given in FIG. 31) and Gag-specific IFN-γ ELISPOTs in DNA/MVA-vaccinated (open bars) and MVA-only (hatched bars) macaques at various times before and after challenge (FIG. 29B). Three pools of 10-13 Gag peptides (22-mers overlapping by 12) were used for the analyses. The numbers above data bars represent the geometric mean for the ELISPOTs within each group. The numbers at the bottom of the graph designate individual animals. #, data not available. *, less than 20 SFU. NA, data not available for group. Data for the Gag-Pol-Env groups are for the group that received 2.5 mg of DNA as an intradermal prime in Amara et al., Science 292:69-74, 2001, the findings of which are reproduced herein).

FIGS. 30A and 30B are graphs illustrating temporal antibody responses. Temporal patterns of anti-Env binding, anti-Env neutralizing, and anti-Gag binding antibodies are examined. Micrograms of total SIV239 Gag or 89.6 Env antibody were determined using enzyme linked immunosorbent assays (ELISAs). The titers of neutralizing antibody for SHIV-89.6 and SHIV-89.6P were determined using MT-2 cell killing and neutral red staining (Montefiori et al., J. Clin. Microbiol. 26:231-235, 1988). Neutralization titers are the reciprocal of the serum dilution giving 50% neutralization of the indicated viruses grown in human PBMC. Symbols for animals are the same as in FIG. 31. FIG. 30B illustrates avidity of anti-Env binding antibody at 2 weeks post challenge. GMT, geometric mean titer.

FIGS. 31A-31D are graphs illustrating temporal viral loads and CD4 counts after challenge of vaccinated and control animals. A, Geometric mean viral loads and B, geometric mean CD4 counts. C, Viral loads and D, CD4 counts for individual animals in the vaccine and control groups. The key to animal numbers is presented in panel D. Assays for the first 12 weeks for the Gag-Pol-Env groups had a background of 1000 copies of RNA per ml of plasma. Animals with loads below 1000 were scored with a load of 500. For all other assays, the background for detection was 300 copies of RNA/ml, and animals with levels of virus below 300 scored at 300. t represents the death of an animal. GM, geometric mean titers of each group.

FIGS. 32A and 32B illustrate viral loads and infected cells in the peripheral blood at 2 weeks post challenge (see the protocol described in Example 20). Intracellular p27 staining PBMC were fixed and stained for intracellular Gag, CD3 and CDS. Cells were gated on lymphocytes followed by CD3+, CDS− and analyzed for Gag. The frequencies in the graph represent Gag positive cells as the % of total CD4 cells. Representative data are shown for each group: animal #3 (pre-challenge), animals #3, #45 and #26 (postchallenge) (FIG. 32A). Comparison of viral loads and number of infected cells at 2 weekspost challenge. Geometric means for viral RNA copies and percent infected CD4 cells are represented as horizontal bars on the respective graphs. Filled symbols represent the DNA/MVA-vaccinated animals and the open symbols represent the MVA-only vaccinated animals. The diagonal lines represent the trend lines for the DNA/MVA vaccinated animals (solid) and the MVA-only vaccinated animals (dashed) (FIG. 32B).

FIG. 33 is a series of graphs illustrating the geometric mean titers (GMT) for antibody raised by recombinant and wild type MVA (uppermost panel); the titers for antivaccinia antibody for the five individual monkeys used to test the wild type MVA for the ability to raise anti-vaccinia antibody (middle panel); and the titers of vaccinia virus antibody for the six individual macaques used to test the MVA/HIV-48 for the ability to raise anti-vaccinia antibody (lower panel).

FIG. 34 is a schematic representation of vaccine inserts pGA/JS2, pGA2/JS7, and PGA2/JS7. 1. Protease mutation D25A, in the catalytic site, eliminates protease activity. The start site of Vpu in pGA/JS7.1 was mutated along with a downstream ATG to eliminate translation of Vpu.

FIG. 35A is a photograph of a Western blot performed to examine Gag expression nin DNA vaccine candidates. Tissue culture supematants and cell lysates were harvested 40 hours post transfection with 300 ng of plasmid. Gag expression is depicted by westernblot (A). JS8 expresses Gag from a codon optimized gene and is shown for comparative purposes only. FIG. 35B illustrates Env protein levels (determined by ELISA).

FIG. 36 is an electron micrograph of intracellular aggregation of HIV-1 proteins produced from pGA2/JS2 in transiently transfected 293T cells. Normal virus particles are typically 90-130 nm in diameter and are produced by budding at the cell surface.

FIG. 37 illustrates production of VLPs produced from pGA2/JS7 in transiently transfected 293T cells. Particles are approximately 100 nm in diameter. The arrows highlight the presence of HIV-1 Env glycoprotein incorporated into the VLP by binding of anti-HIV-1 env antibody conjugated to gold particles.

FIG. 38 is a schematic representation of clade AG vaccine inserts pGA/1 C2,pGA1/IC25, pGA1/IC48, and pGA1/IC90. The original genetic material was derived from a patient isolate from Ivory Coast. Protease mutation D25A is in the catalytic site and eliminates protease activity. The G48V and L48M mutations are derived from protease mutations found in drug resistant isolates and only partially inhibit protease function.

FIG. 39. Gag and Env expression of clade AG DNA vaccine constructs. Tissue culture supernatants and cell lysates were harvested at 48 hours post transfection and analyzed by ELISA.

FIGS. 40A-40D. Aggregate and particle formation from clade AG DNA vaccine constructs. (A) Clade AG with wt protease; (B) Same constructs as panel A with addition of inhibitor of viral protease added to culture; (C) pGA1/IC48; and (D) pGA1/IC90. FIGS. 41A-41F show the sequence of various IC inserts (clade AG).

### DETAILED DESCRIPTION

This invention encompasses a variety of types of vectors, each of which may include one or more nucleic acid sequences that encode an antigen from a pathogen (i.e., each of which may have a vaccine insert), and methods of using these vectors, alone or in combination with one another, to either immunize patients against the pathogen(s) from which the antigen(s) were obtained (thereby reducing the patient's risk of becoming infected) or to treat patients who have already become infected. The immunization methods can elicit both cell-mediated and humoral immune responses that may substantially prevent the infection or limit its extent or impact on the patient's health. Immunization can result in protection against subsequent challenge by the pathogen; a patient (e.g., a human or other mammal, such as a domesticated animal) is immunized if they mount an immune response that protects them (partially or totally) from the manifestations of infection (i. e., disease) caused by a pathogen. Thus, an immunized patient will not be infected by the pathogen or will be infected to a lesser extent than one would expect in the absence of immunization.

The vaccines, regardless of the pathogen they are directed against, can include a nucleic acid vector (e.g., a plasmid) that contains a terminator sequence (i.e., a nucleotide sequence that substantially inhibits transcription, the process by which RNA molecules are formed upon DNA templates by complementary base pairing. A useful terminator sequence is the lambda T0 terminator sequence. The terminator sequence is positioned within the vector in (a) the same orientation as, and in-frame with, a selectable marker gene (i.e., the terminator sequence and the selectable marker gene are operably linked) and in (b) the opposite orientation from a sequence encoding an antigen when that sequence is inserted into the vector's cloning (or multi-cloning) site. By preventing read through from the selectable marker into the vaccine insert as the plasmid replicates in prokaryotic cells, the terminator stabilizes the insert as the bacteria grow and the plasmid replicates.

Selectable marker genes are known in the art and include, for example, genes encoding proteins that confer antibiotic resistance on a cell in which the marker is expressed (e.g., resistance to kanamycin or ampicillin). The selectable marker is so-named because it allows one to select cells by virtue of their survival under conditions that, absent the marker, would destroy them. The selectable marker, the terminator sequence, or both (or parts of each or both) can be, but need not be, excised from the plasmid before it is administered to a patient. Similarly, plasmid vectors can be administered in a circular form, after being linearized by digestion with a restriction endonuclease, or after some of the vector “backbone” has been altered or deleted.

The nucleic acid vectors can also include an origin of replication (e.g., a prokaryotic on) and a transcription cassette that, in addition to containing one or more restriction endonuclease sites, into which a vaccine insert can be cloned, optionally includes a promoter sequence and a polyadenylation signal. Promoters known as strong promoters can be used and may be preferred. One such promoter is the cytomegalo virus (CMV) intermediate early promoter, although other (including weaker) promoters may be used without departing from the scope the present invention. Similarly, strong polyadenylation signals may be selected (e.g., the signal derived from a bovine growth hormone (BGH) encoding gene, or a rabbit˜globin polyadenylation signal (Bohm et al., J. Immunol. Methods 193:29-40, 1996; Chapman et al., Nucl. Acids Res. 19:3979-3986, 1991; Hartikka et al., Hum. Gene Therapy 7:1205-1217, 1996; Manthorpe et al., Hum. Gene Therapy 4:419-431, 1993; Montgomery et al., DNA Cell Biol. 12:777-783, 1993)).

The vectors can further include a leader sequence (a leader sequence that is a synthetic homolog of the tissue plasminogen activator gene leader sequence (tPA) is optional in the transcription cassette) and/or an intron sequence such as a cytomegalovirus intron A. The presence of intron A increases the expression of many antigens from RNA viruses, bacteria, and parasites, presumably by providing the expressed RNA with sequences which support processing and function as an eukaryotic mRNA. It will be appreciated that expression also may be enhanced by other methods known in the art including, but not limited to, optimizing the codon usage of prokaryotic mRNAs for eukaryotic cells (Andre et al., J. Viral. 72: 1497-1503, 1998; Uchijima et al., J Immunol. 161:5594-5599, 1998). Multi-cistronic vectors may be used to express more than one immunogen or an immunogen and an immunostimulatory protein (Iwasaki et al., J Immunol. 158:4591-4601, 1997a; Wild et al., Vaccine 16:353-360, 1998).

The vectors of the present invention differ in the sites that can be used for accepting vaccine inserts and in whether the transcription cassette includes intron A sequences in the CMVIE promoter (accordingly, one of ordinary skill in the art may modify the insertion site(s) for vaccine insert(s) without departing from the scope of the invention). Both intron A and the tPA leader sequence have been shown in certain instances to supply a strong expression advantage to vaccine inserts (Chapman et al., Nucleic Acids Research 19:3979-3986, 1991).

As described further below, the vectors of the present invention can be administered with an adjuvant, including a genetic adjuvant. Accordingly, the nucleic acid vectors can optionally include one or more C3d gene sequences (e.g., 1-3 (or more) C3d gene sequences).

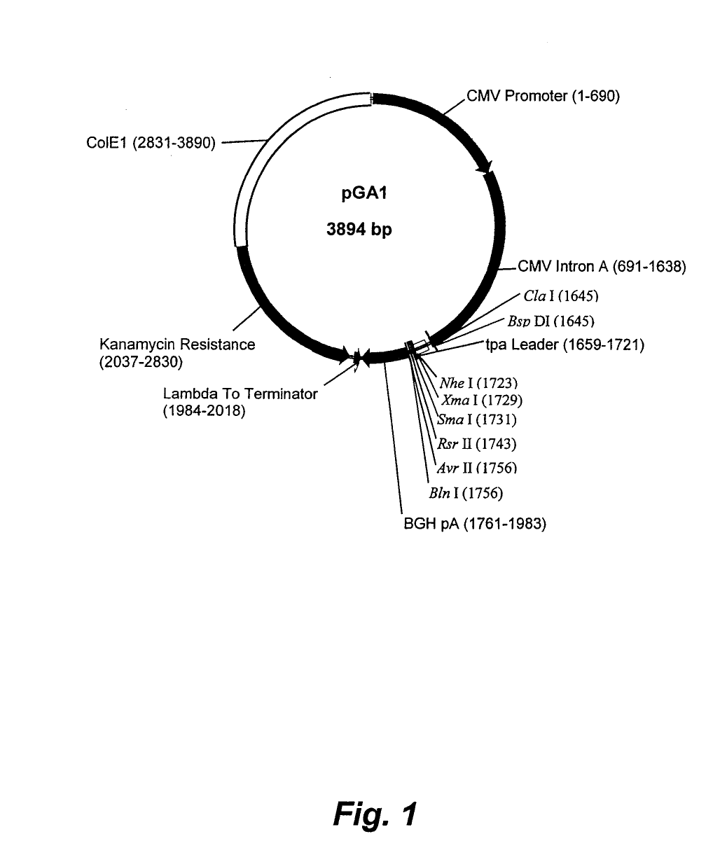

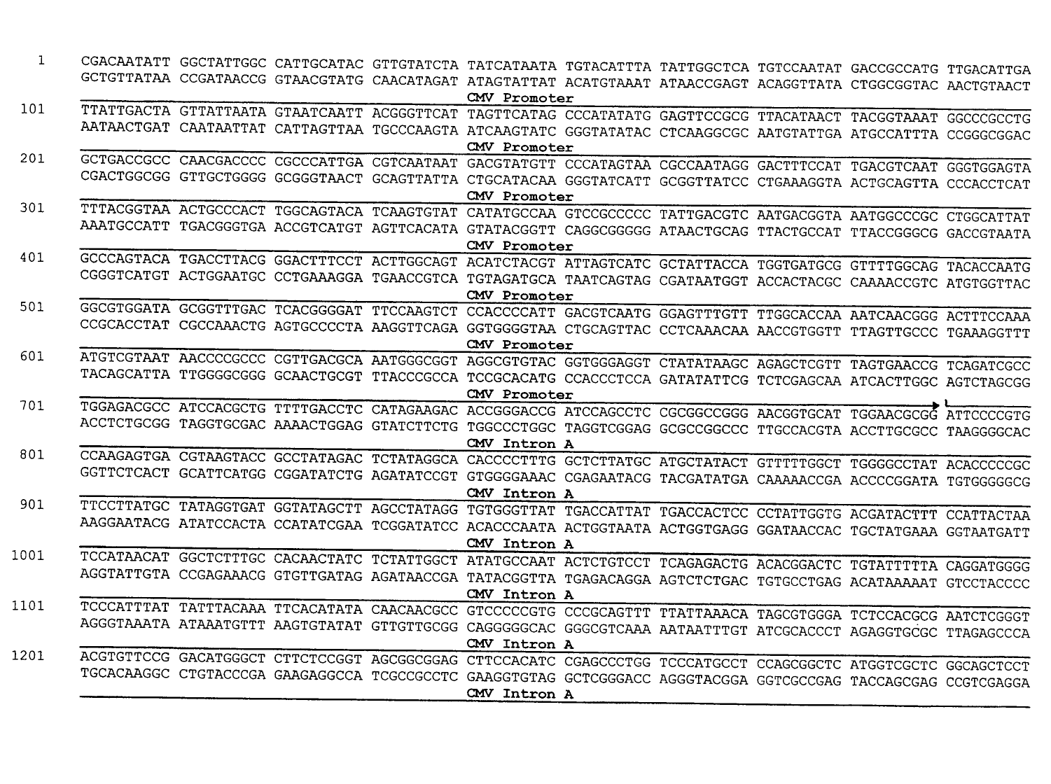

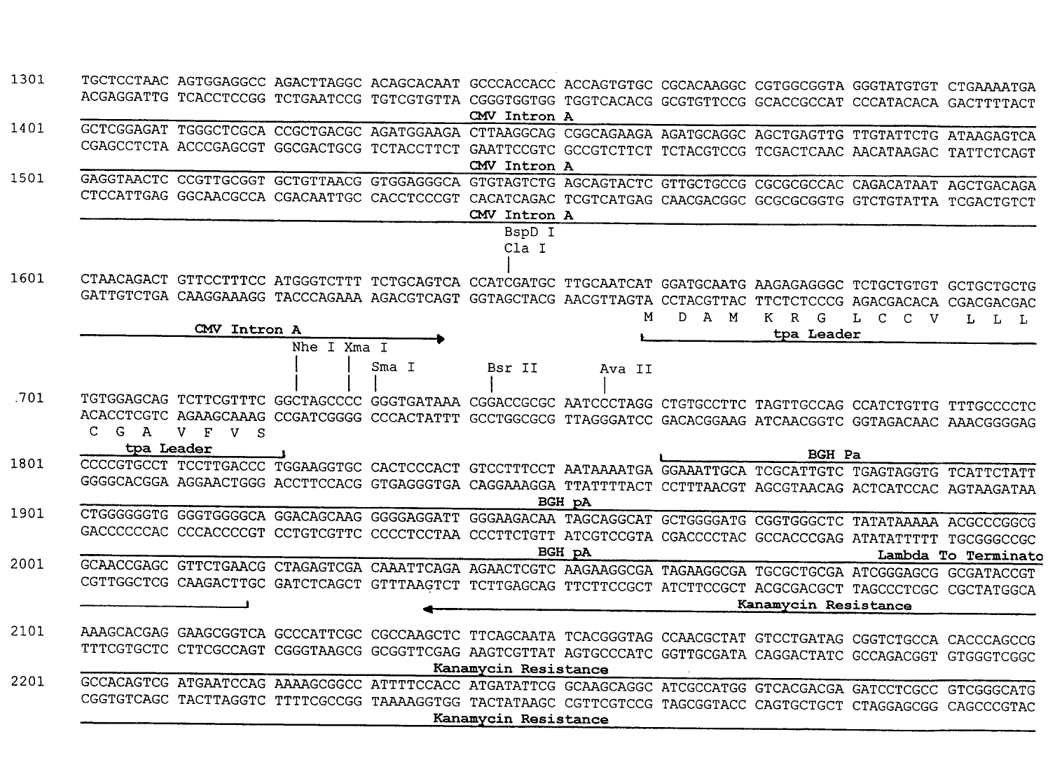

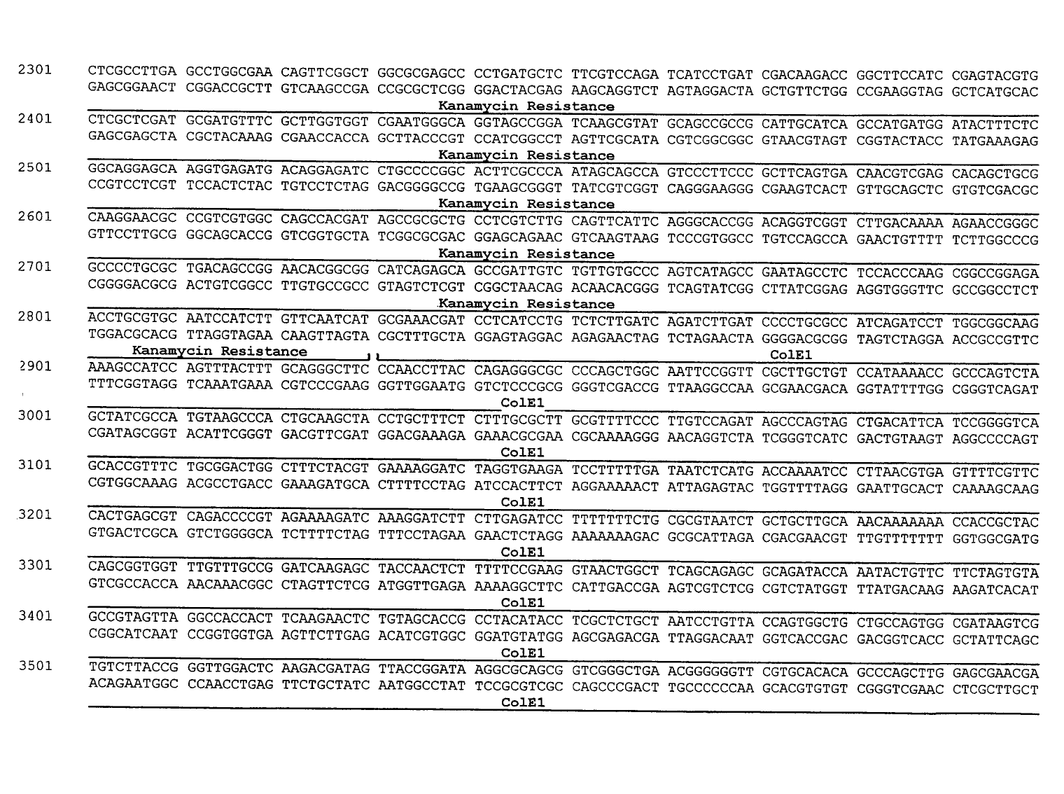

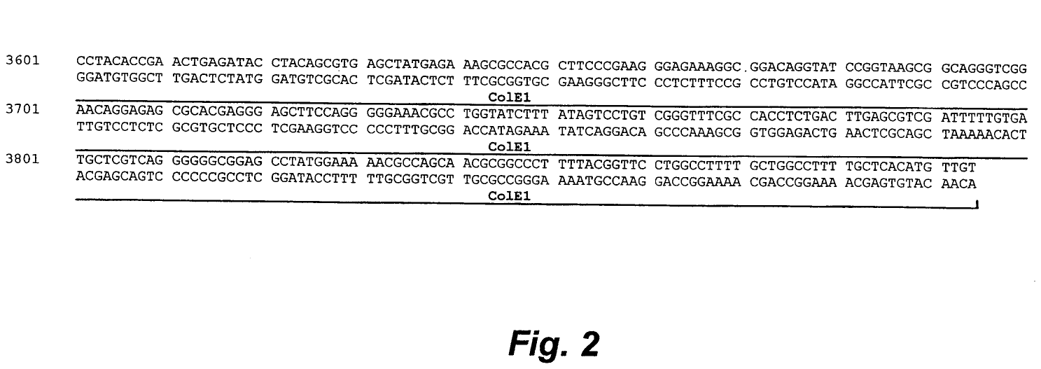

In the event the vector administered is a pGA vector, it can comprise the sequence of, for example, SEQ ID NO:1, SEQ ID NO:2, or SEQ ID NO:3. The pGA vectors are described in more detail here (see also Examples 1-3). pGA1 is a 3894 by plasmid. pGA1 comprises a promoter (bp 1-690), the CMV-intron A (bp 691-1638), a synthetic mimic of the tP A leader sequence (bp 1659-1721), the bovine growth hormonepolyadenylation sequence (bpl 761-1983), the lambda T0 terminator (bp 1984-2018), the kanamycin resistance gene (bp 2037-2830) and the ColEI replicator (bp 2831-3890). The DNA sequence of the pGA1 construct (SEQ ID NO: 1) is shown in FIG. 2. In FIG. 1, the indicated restriction sites are useful for the cloning of vaccine inserts. The Cla I or BspD I sites are used when the 5′ end of a vaccine insert is cloned upstream of the tPA leader. The Nhe I site is used for cloning a sequence in frame with the tPA leader sequence. The sites listed between Sma I and Bln I are used for cloning the 3′ terminus of a vaccine insert.

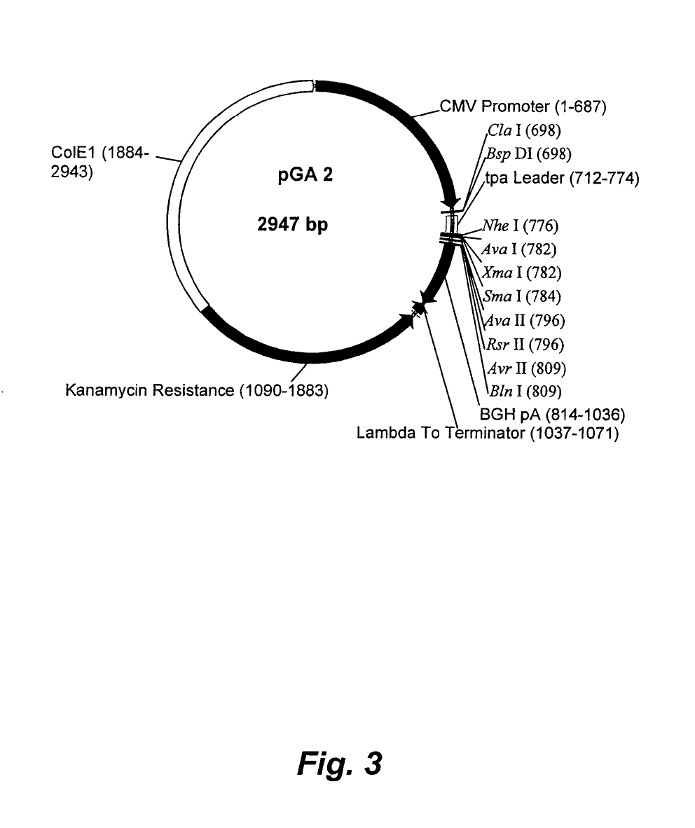

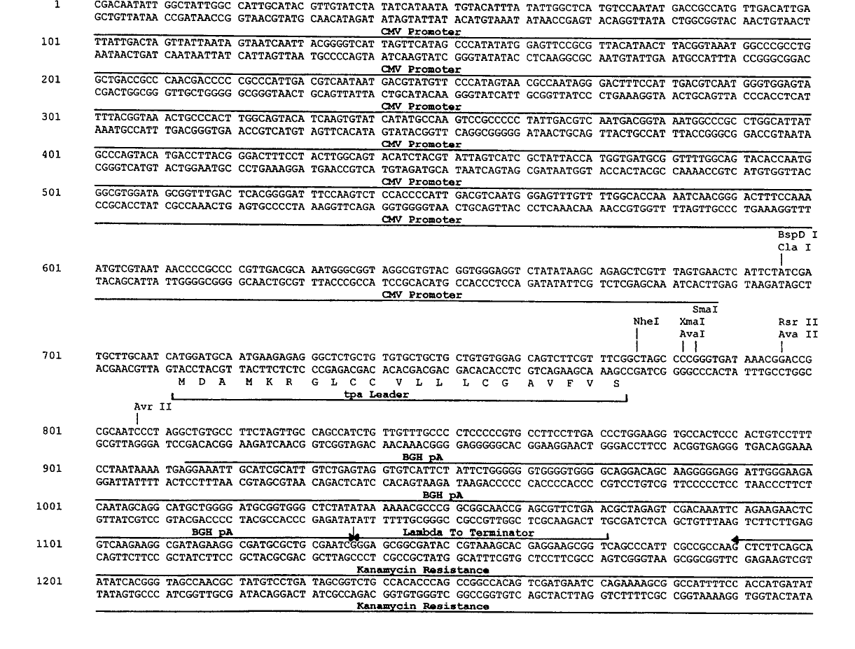

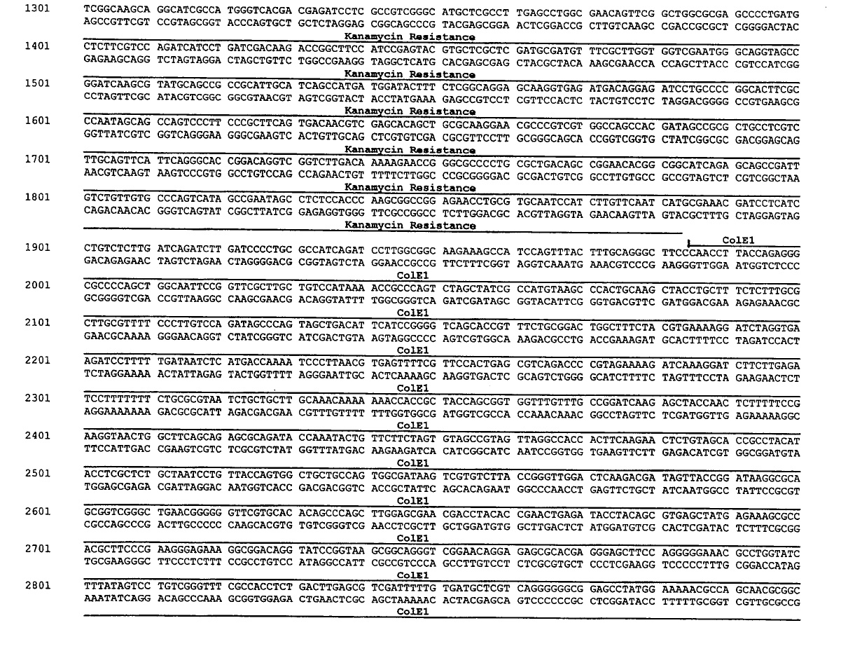

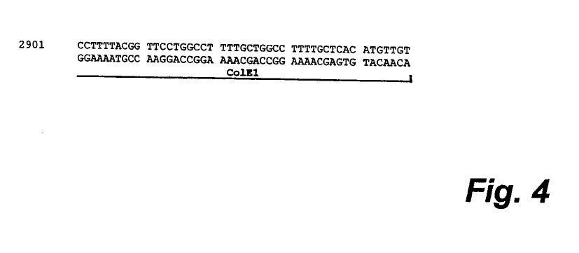

pGA2 is a 2947 by plasmid lacking the 947 by of intron A sequences found in pGA1. pGA2 is the same as pGA1, except for the deletion of intron A sequences. pGA2 is valuable for cloning sequences which do not require an upstream intron for efficient expression, or for cloning sequences in which an upstream intron might interfere with the pattern of splicing needed for good expression. FIG. 3 presents a schematic map of pGA2 with useful restriction sites for cloning vaccine inserts. FIG. 4 shows the DNA sequence of pGA2 (SEQ ID NO: 2). The use of restriction sites for cloning vaccine inserts into pGA2 is the same as that used for cloning fragments into pGA1.

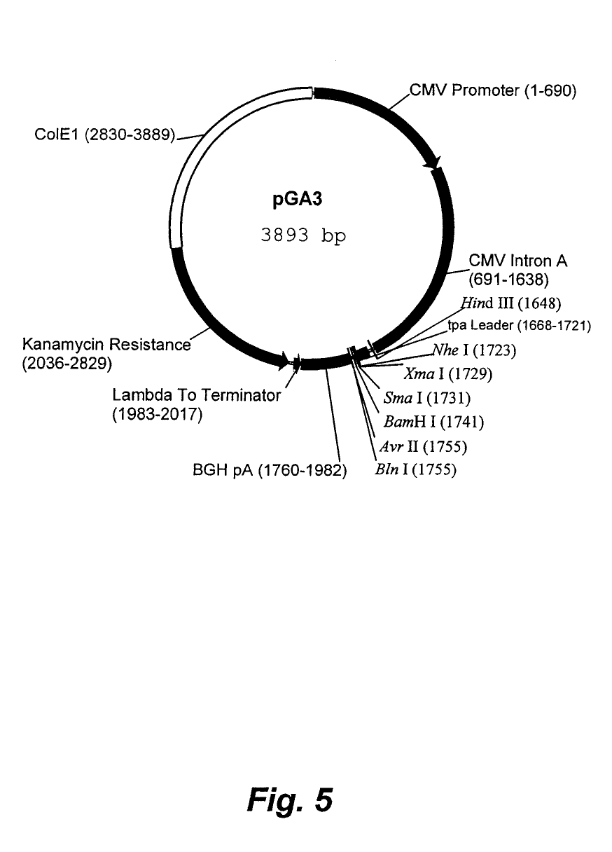

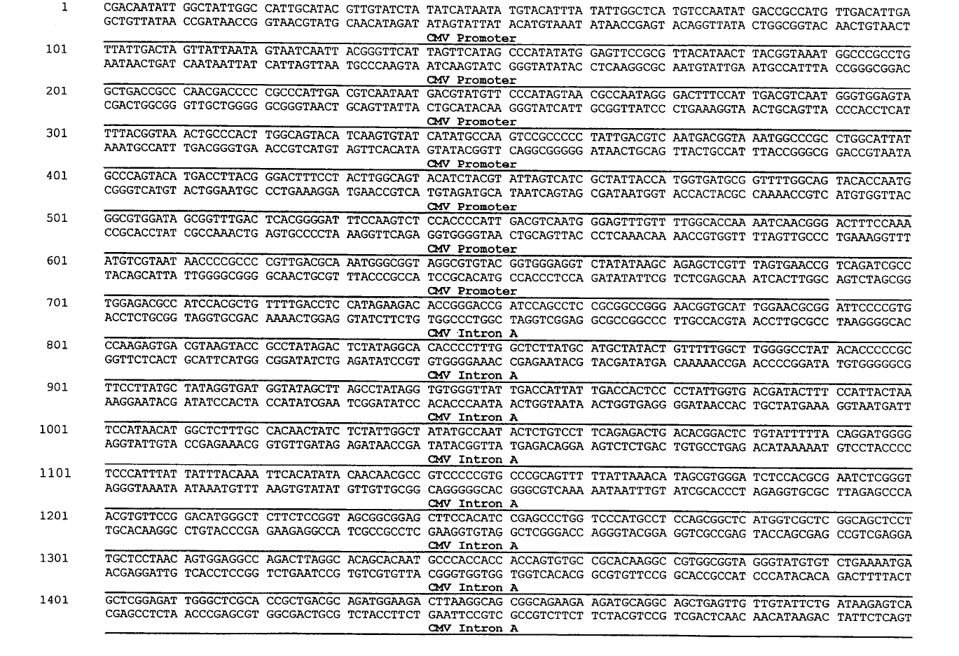

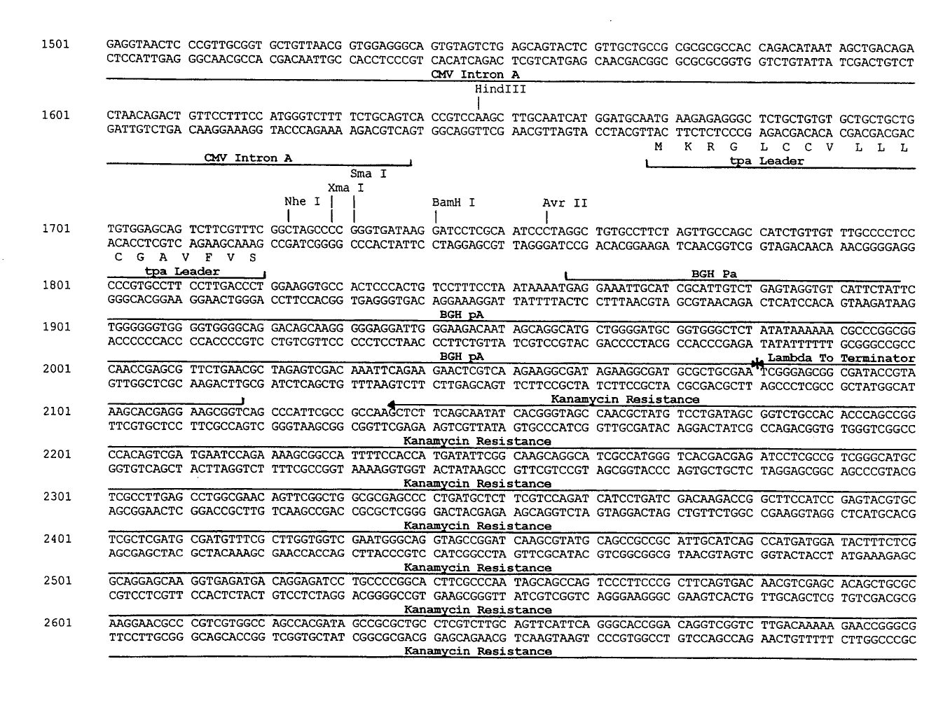

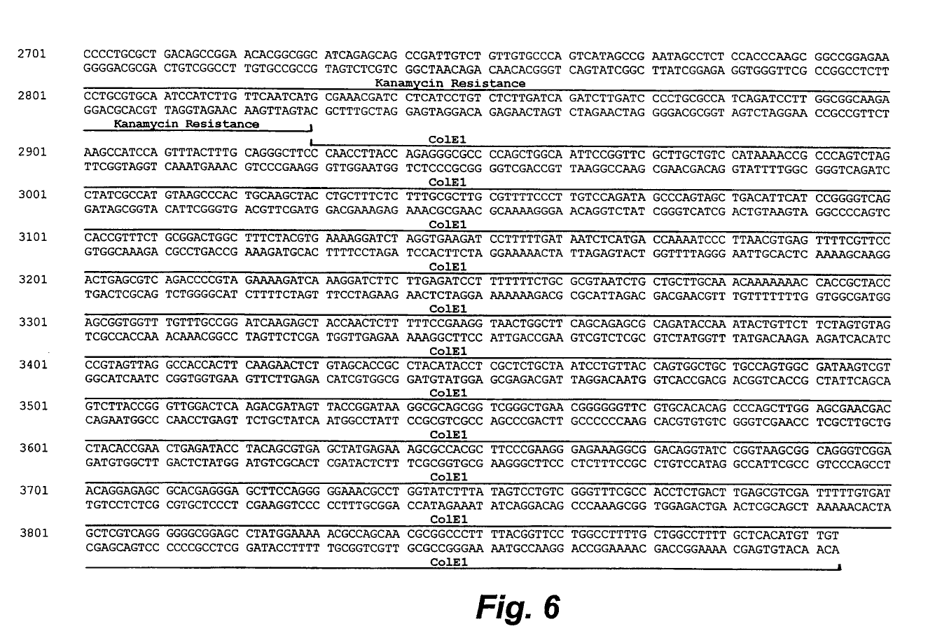

pGA3 is a 3893 by plasmid that contains intron A. pGA3 is the same as pGA1 except for the cloning sites available for the introduction of vaccine inserts. In pGA3,inserts cloned upstream of the tPA leader sequence use a Hind III site. Sequences cloned downstream from the tPA leader sequence use sites between the Sma I and the Bin I sites. In pGA3, these sites include a BamH I site. FIG. 5 presents the schematic map for pGA3. FIG. 6 shows the DNA sequence of vaccine vector pGA3 (SEQ ID NO: 3).

pGA plasmids having sequences that differ from those disclosed herein are also within the scope of the invention so long as the plasmids retain substantially all of the characteristics necessary to be therapeutically effective (e.g., one can substitute nucleotides (particularly where the substitution does not alter the protein encoded), add nucleotides, or delete nucleotides so long as the plasmid, when administered to a patient, induces or enhances an immune response against a given pathogen).

The nucleic acid vectors of the invention, including pGA1, pGA2, and pGA3, can further comprise a nucleic acid sequence that encodes at least one antigen (which may also be referred to as an immunogen) obtained from, or derived from, at least one pathogen. The pathogen can be any virus, bacteria, parasite or fungi that generats a pathological condition in an animal. The virus can be, for example, a herpesvirus, an influenza virus, a orthomyxovirus, a rhinovirus, a picornavirus, an adenovirus, a paramyxovirus, a coronavirus, a rhabdovirus, a togavirus, a flavivirus, a bunyavirus, a rubella virus, a reovirus, a measles virus, a hepadna virus, an Ebola virus, or a retrovirus (including a human immunodeficiency virus; including all clades of HIV-1 and HIV-2 and modifications thereof). The bacteria can be, for example, a mycobacterium (e.g., M. tuberculosis, which causes tuberculosis or M. leprae, which causes leprosy), aspirochete, a rickettsia, a chlamydia, or a mycoplasma. The parasite can be, for example, a parasite that causes malaria, and the fungus can be, for example, a yeast or mold. One of ordinary skill in the art will recognize that the methods described herein can be used to generate protective or therapeutic immune responses against many other pathogens.

The antigen (or immunogen) maybe a structural component of the pathogen; the antigen (or immunogen) may be glycosylated, myristoylated, or phosphorylated; the antigen (or immunogen) may be one that is expressed intracellularly, on the cell surface, or secreted (antigens that are not normally secreted may be linked to a signal sequence that directs secretion). More specifically, where the antigen is obtained from, or derived from, an immunodeficiency virus, the antigen can be all, or an antigenic portion of, Gag, gp120, Pol, Env, Tat, Rev, Vpu, Nef, Vif, Vpr, or a VLP (e.g., a polypeptide derived from a VLP, including an Env-defective HIV VLP. Plasmids useful in preventing or treating AIDS include those that express the JS2 clade B HIV-1 VLP (SEQ ID NO: 4) and those that express the JS5 clade B HIV-1 Gag-pol insert (SEQ ID NO: 5). Sequences from other HIV clades, particularly clade AG (exemplified by sequences designated herein as “IC”) may also be used as vaccine inserts to immunize or treat patients in regions of the world where clades other than clade B predominate.

Where the antigen is obtained from, or derived from, the virus that causes measles, the antigen can be all, or an antigenic portion of, measles fusion protein, nucleoprotein, or hemagglutinin (hemagglutinin may also be selected from an influenza virus). Antigens directed against any pathogenic condition may contain a mutation, so long as they retain the ability to induce or enhance an immune response that confers a protective or therapeutic benefit on the patient.

The methods of the invention (e.g., methods of eliciting an immune response in a patient) can be carried out by administering to the patient a therapeutically effective amount of a first physiologically acceptable composition comprising a vector having one or more of the characteristics of the pGA constructs described above (e.g., a selectable marker gene, a prokaryotic origin of replication, a termination sequence (e.g., the lambda T0 terminator) and operably linked to the selectable gene marker, and a eukaryotic transcription cassette comprising a promoter sequence, a nucleic acid insert encoding at least one antigen derived from a pathogen, and a polyadenylation signal sequence). A therapeutically effective amount of the first vector can be administered by an intramuscular, intradermal or subcutaneous route, together with a physiologically acceptable carrier, diluent, or excipient, and, optionally, an adjuvant. These components can be readily selected by one of ordinary skill in the art, regardless of the precise nature of the antigens incorporated in the vaccine or the vector by which they are delivered. When the vector comprises SEQ ID NO: 1, nucleotides from positions 1643 to 1721 can be omitted; when the vector comprises SEQ ID NO: 2, nucleotides from position 689 to nucleotide position 774 can be omitted.

The immunodeficiency virus vaccine inserts of the present invention were designed to express non-infectious VLPs (a term that can encompass true VLPs as well as aggregates of viral proteins) from a single DNA This was achieved using the subgenomic splicing elements normally used by immunodeficiency viruses to express multiple gene products from a single viral RNA. Important to the subgenomic splicing patterns are (i) splice sites and acceptors present in full length viral RNA, (ii) the Rev responsive element (RRE) and (iii) the Rev protein. The splice sites in retroviral RNAs use the canonical sequences for splice sites in eukaryotic RNAs. The RRE is an approximately 200 bp RNA structure that interacts with the Rev protein to allow transport of viral RNAs from the nucleus to the cytoplasm. In the absence of Rev, the approximately 10 kb RNA of immunodeficiency virus undergoes splicing to the mRNAs for the regulatory genes Tat, Rev, and Nef. These genes are encoded by exons present between RT and Env and at the 3′ end of the genome. In the presence of Rev, the singly spliced mRNA for Env and the unspliced mRNA for Gag and Pol are expressed in addition to the multiply spliced mRNAs for Tat, Rev, and Nef.

The expression of non-infectious VLPs from a single DNA affords a number of advantageous features to an immunodeficiency virus vaccine. The expression of a number of proteins from a single DNA affords the vaccinated host the opportunity to respond to the breadth of T- and B cell epitopes encompassed in these proteins. The expression of proteins containing multiple epitopes affords the opportunity for the presentation of epitopes by diverse histocompatibility types. By using whole proteins, one offers hosts of different histocompatibility types the opportunity to raise broad-based T cell responses. Such may be essential for the effective containment of immunodeficiency virus infections, whose high mutation rate supports ready escape from immune responses (Evans et al., Nat. Med. 5:1270-1276, 1999; Poignard et al., Immunity 10:431-438, 1999, Evans et al., 1995). Just as in drug therapy, multi-epitope T cell responses that require multiple mutations for escape will provide better protection than single epitope T-cell responses that require only a single mutation for escape.

Antibody responses are often best primed by multi-valent vaccines that present an ordered array of an epitope to responding B cells (Bachmann et al., Ann. Rev. Immunol. 15:235-270, 1997). Virus-like particles, by virtue of the multivalency of Env in the virion membrane, will facilitate the raising of anti-Env antibody responses. These particles will also present non-denatured and normal forms of Env to the immune system.

Immunogens can also be engineered to be more or less effective for raising antibody or Tc by targeting the expressed antigen to specific cellular compartments. For example, antibody responses are raised more effectively by antigens that are displayed on the plasma membrane of cells, or secreted therefrom, than by antigens that are localized to the interior of cells (Boyle et al., Int. Immunol. 2.:1897-1906, 1997; Inchauspe et al., DNA Cell. Biol. 16:185-195, 1997). Tc responses maybe enhanced by using N-terminal ubiquitination signals which target the DNA-encoded protein to the proteosome causing rapid cytoplasmic degradation and more efficient peptide loading into the MHC I pathway (Rodriguez et al., J. Viral. 71:8497-8503, 1997; Tobery et al., J. Exp. Med. 185:909-920, 1997; Wu et al., J. Immunol. 159:6037-6043, 1997). For a review on the mechanistic basis for DNA-raised immune responses, refer to Robinson and Pertmer, Advances in Virus Research, vol. 53, Academic Press (2000).

The effects of different conformational forms of proteins on antibody responses, the ability of strings of MHC I epitopes (minigenes) to raise Tc responses, and the effect of fusing an antigen with immune-targeting proteins have been evaluated using defined inserts. Ordered structures such as virus-like particles appear to be more effective than unordered structures at raising antibody (Fomsgaard et al., Scand. J. Immunol. 47:289-295, 1998). This is likely to reflect the regular array of an immunogen being more effective than a monomer of an antigen at cross-linking Ig-receptors and signaling a B cell to multiply and produce antibody. Recombinant DNA molecules encoding a string of MHC epitopes from different pathogens can elicit Tc responses to a number of pathogens (Hanke et al., Vaccine 16:426-435, 1998). These strings of Tc epitopes are most effective if they also include a Th epitope (Maecker et al., J. Immunol. 161:6532-6536, 1998; Thomson et al., J. Immunol. 160:1717-1723, 1998).

Another approach to manipulating immune responses is to fuse immunogens to immuno targeting or immunostimulatory molecules. To date, the most successful of these fusions have targeted secreted immunogens to antigen presenting cells (APC) or lymph nodes (Boyle et al., Nature 392:408-411, 1998). Fusion of a secreted form of human IgG with CTLA-4 increased antibody responses to the IgG greater than 1000-fold and changed the bias of the response from complement (C′-) dependent to C′-independent antibodies.

Fusions of human IgG with L-selectin also increased antibody responses but did not change the C′-binding characteristics of the raised antibody. The immunogen fused with L-selectin was presumably delivered to lymph nodes by binding to the high endothelial venules, which serve as portals. Fusions between antigens and cytokine cDNAs have resulted in more moderate increases in antibody, Th, and Tc responses (Hakim et al., J. Immunol. 157:5503-5511, 1996; Maecker et al., Vaccine 15:1687-1696, 1997). IL-4-fusions have increased antibody responses, whereas IL-12 and IL-1˜have enhanced T-cell responses.

Two approaches to DNA delivery are injection of DNA in saline using a hypodermic needle or gene gun delivery of DNA-coated gold beads. Saline injections deliver DNA into extracellular spaces, whereas gene gun deliveries bombard DNA directly into cells. The saline injections require much larger amounts of DNA (100-1000 times more) than the gene gun (Fynan et al., Proc. Natl. Acad. Sci. USA 90:11478-11482, 1993). These two types of delivery also differ in that saline injections bias responses towards type 1 T-cell help, whereas gene gun deliveries bias responses towards type 2 T-cell help (Feltquate et al., J. Immunol. 158:2278-2284, 1997; Pertmer et al., J. Viral. 70:6119-6125, 1996). DNAs injected in saline rapidly spread throughout the body. DNAs delivered by the gun are more localized at the target site. Following either method of inoculation, extracellular plasmid DNA has a short half life of about 10 minutes (Kawabata et al., Pharm. Res. 12:825-830, 1995; Lew et al., Hum. Gene Ther. §:553, 1995). Vaccination by saline injections can be intramuscular (i.m.) or intradermal (i.d.) (Fynan et al., 1993).

Although intravenous and subcutaneous injections have met with different degrees of success for different plasmids (Bohm et al., Vaccine 16:949-954, 1998; Fynan et al., 1993), intraperitoneal injections have not met with success (Bohm et al., 1998; Fynan et al., 1993). Gene gun deliveries can be administered to the skin or to surgically exposed muscle. Methods and routes of DNA delivery that are effective at raising immune responses in mice are effective in other species.

Immunization by mucosal delivery of DNA has been less successful than immunizations using parenteral routes of inoculation. Intranasal administration of DNA in saline has met with both good (Asakura et al., Scand. J. Immunol. 46:326-330, 1997; Sasaki et al., Infect. Immun. 66:823-826, 1998b) and limited (Fynan et al., 1993) success. The gene gun has successfully raised IgG following the delivery of DNA to the vaginal mucosa (Livingston et al., Ann. New York Acad. Sci. 772:265-267, 1995). Some success at delivering DNA to mucosal surfaces has also been achieved using liposome (McCluskie et al., Antisense Nucleic Acid Drug Dev. 8:401-414, 1998), microspheres (Chen et al., J Virol. 72:5757-5761, 1998a; Jones et al., Vaccine 15:814-817, 1997) and recombinant Shigella vectors (Sizemore et al., Science 270:299-302, 1995; Sizemore et al., Vaccine 12:804-807, 1997).

The dose of DNA needed to raise a response depends upon the method of delivery, the host, the vector, and the encoded antigen. The most profound effect is seen for the method of delivery. From 10 μg to 1 mg of DNA is generally used for saline injections of DNA, whereas from 0.2 μg to 20 μg of DNA is used for gene gun deliveries of DNA. In general, lower doses of DNA are used in mice (10-100 μg for saline injections and 0.2 μg to 2 μg for gene gun deliveries), and higher doses in primates (100 μg to 1 mg for saline injections and 2 μg to 20 μg for gene gun deliveries). The much lower amount of DNA required for gene gun deliveries reflect the gold beads directly delivering DNA into cells.

An example of the marked effect of an antigen on the raised response can be found in studies comparing the ability to raise antibody responses in rabbits of DNAs expressing the influenza hemagglutinin or an immunodeficiency virus envelope glycoprotein (Env) (Richmond et al., J Virol. 72:9092-9100, 1998). Under similar immunization conditions, the hemagglutinin-expressing DNA raised long lasting, high avidity, high titer antibody (˜100 μg per ml of specific antibody), whereas the Env-expressing DNA raised only transient, low avidity, and low titer antibody responses (<IO μg per ml of specific antibody). These differences in raised antibody were hypothesized to reflect the hemagglutinin being a T-dependent antigen and the highly glycosylated immunodeficiency virus Env behaving as a T-independent antigen.

Both protein and recombinant viruses have been used to boost DNA-primed immune responses. Protein boosts have been used to increase neutralizing antibody responses to the HIV-1 Env. Recombinant pox virus boosts have been used to increase both humoral and cellular immune responses. For weak immunogens, such as the immunodeficiency virus Env, for which DNA-raised antibody responses are only a fraction of those in naturally infected animals, protein boosts have provided a means of increasing low titer antibody responses (Letvin et al., Proc. Natl. Acad. Sci USA 94:9378-9383, 1997; Richmond et al., 1998). In a study in rabbits, the protein boost increased both the titers of antibody and the avidity and the persistence of the antibody response (Richmond et al., 1998). Consistent with a secondary immune response to the protein boost, DNA primed animals showed both more rapid increases in antibody, and higher titers of antibody following a protein boost than animals receiving only the protein. However, by a second protein immunization, the kinetics and the titer of the antibody response were similar in animals that had, and had not, received DNA priming immunizations.

Recombinant pox virus boosts have proved to be a highly successful method of boosting DNA-primed CD8+ cell responses (Hanke et al., Vaccine 16:439-445, 1998a; Kent et al., J. Virol. 72:10180-10188, 1998; Schneider et al., Nat. Med 1_{—}:397-402, 1998). Following pox virus boosters, antigen-specific CD8+ cells have been increased by as much as 10-fold in DNA primed mice or macaques. Studies testing the order of immunizations reveal that the DNA should be delivered first (Schneider et al., 1998). This has been hypothesized to reflect the DNA focusing the immune response on the desired immunogens. The larger increases in CDS+ cell responses following pox virus boosts has been hypothesized to reflect both the larger amount of antigen expressed by the pox virus vector, as well as pox virus-induced cytokines augmenting immune responses (Kent et al., J. Virol. 72:10180-10188, 1998; Schneider et al., Nat. Med. 4:397-402, 1998).

Here, a number of different pox viruses can be used either alone (i.e., without a nucleic acid or DNA prime) or as the boost component of a vaccine regimen. MV A has been particularly effective in mouse models (Schneider et al., 1998). MVA is a highly attenuated strain of vaccinia virus that was developed toward the end of the campaign for the eradication of smallpox, and it has been safety tested in more than 100,000 people (Mahnel et al., Berl. Munch Tierarztl Wochenschr 107:253-256, 1994; Mayr et al. Zentralbl. Bakteriol. 167:375-390, 1978). During over 500 passages in chicken cells, MVA lost about 10% of its genome and the ability to replicate efficiently in primate cells. Despite its limited replication, MVA has proved to be a highly effective expression vector (Sutter et al., Proc. Natl. Acad. Sci. USA 89:10847-10851, 1992), raising protective immune responses in primates for parainfluenza virus (Durbin et al. J. Infect.Dis. 179:1345-1351, 1999), measles (Stittelaar et al. J. Viral. 74:4236-4243, 2000), and immunodeficiency viruses (Barouch et al., J. Virol. 75:5151-5158, 2001; Ourmanov et al., J. Viral. 74:2740-2751, 2000). The relatively high immunogenicity of MVA has been attributed in part to the loss of several viral anti-immune defense genes (Blanchard et al., J. Gen. Viral. 79:1159-1167, 1998).

Responses raised by a DNA prime followed by pox virus boost can be highly effective at raising protective cell-mediated immune responses. In mice, intramuscular injections of DNA followed by recombinant pox boosts have protected against a malaria challenge (Schneider et al., 1998). In macaques, intradermal, but not gene gun DNA primes, followed by recombinant pox virus boosters have contained challenges with chimeras of simian and human immunodeficiency viruses (Robinson et al., 1999).

DNA vaccines for immunodeficiency viruses such as HIV-1 encounter the challenge of sufficiently limiting an incoming infection such that the inexorable longterm infections that lead to AIDS are prevented. Complicating this is that neutralizing antibodies are both difficult to raise and specific against particular viral strains (Burton et al., AIDS 11(Suppl A):587-98, 1997; Moore et al., AIDS 9(Suppl A):S117-136, 1995). Given the problems with raising neutralizing antibody, much effort has focused on raising cell-mediated responses of sufficient strength to severely curtail infections. To date, the best success at raising high titers of Tc have come from immunization protocols using DNA primes followed by recombinant pox virus boosters. The efficacy of this protocol has been evaluated by determining the level of specific Tc using assays for cytolytic activity (Kent et al., 1998), by staining with MHC-specific tetramers for specific SIV Gag epitopes and by challenge with SIV s or SHIV s (Hanke, 1999).

A number of salient findings are emerging from preclinical trials using DNA primes and recombinant pox virus boosts. The first is that challenge infections can be contained below the level that can be detected using quantitative RT-PCR analyses for plasma viral RNA (Robinson et al., 1999). The second is that this protection is longlasting and does not require the presence of neutralizing antibody (Robinson et al., 1999). The third is that intradermal DNA priming with saline injections of DNA is superior to gene gun priming for raising protective immunity (P=0.01, Fisher's exact test) (Robinson et al., 1999).

An adjuvant is a substance that is added to a vaccine to increase the vaccine's immunogenicity. The adjuvant used in connection with the vectors described here (whether DNA or viral-based) can be one that slowly releases antigen (e.g., the adjuvant can be a liposome), or it can be an adjuvant that is strongly immunogenic in its own right (these adjuvants are believed to function synergistically). Accordingly, the vaccine compositions described here can include known adjuvants or other substances that promote DNA uptake, recruit immune system cells to the site of the inoculation, or facilitate the immune activation of responding lymphoid cells. These adjuvants or substances include oil and water emulsions, Corynebacterium parvum, Bacillus Calmette Guerin, aluminum hydroxide, glucan, dextran sulfate, iron oxide, sodium alginate, BactoAdjuvant, certain synthetic polymers such as poly amino acids and co-polymers of aminoacids, saponin, REGRESSIN (Vetrepharm, Athens, Ga.), AVRIDINE (N, N-dioctadecyl N′,N′-bis(2-hydroxyethyl)-propanediamine), paraffin oil, and muramyl dipeptide. Genetic adjuvants, which encode immunomodulatory molecules on the same or a coinoculated vector, can also be used. For example, a sequence encoding C3d can be included on a vector that encodes a pathogenic immunogen (such as an HIV antigen) or on a separate vector that is administered at or around the same time as the immunogen is administered.

The compositions described herein can be administered in a variety of ways including through any parenteral or topical route. For example, an individual can be inoculated by intravenous, intraperitoneal, intradermal, subcutaneous or intramuscular methods. fuoculation can be, for example, with a hypodermic needle, needleless delivery devices such as those that propel a stream of liquid into the target site, or with the use of a gene gun that bombards DNA on gold beads into the target site. The vector comprising the pathogen vaccine insert can be administered to a mucosa! surface by a variety of methods including intranasal administration, i.e., nose drops or inhalants, or intrarectal or intravaginal administration by solutions, gels, foams, or suppositories. Alternatively, the vector comprising the vaccine insert can be orally administered in the form of a tablet, capsule, chewable tablet, syrup, emulsion, or the like. In an alternate embodiment, vectors can be administered transdermally, by passive skin patches, iontophoretic means, and the like.

Any physiologically acceptable medium can be used to introduce a vector (whether nucleic acid-based or live-vectored) comprising a vaccine insert into a patient. For example, suitable pharmaceutically acceptable carriers known in the art include, but are not limited to, sterile water, saline, glucose, dextrose, or buffered solutions. The media may include auxiliary agents such as diluents, stabilizers (i. e., sugars (glucose and dextrose were noted previously) and amino acids), preservatives, wetting agents, emulsifying agents, pH buffering agents, additives that enhance viscosity or syringability, colors, and the like. Preferably, the medium or carrier will not produce adverse effects, or will only produce adverse effects that are far outweighed by the benefit conveyed.

The present invention is further illustrated by the following examples, which are provided by way of illustration and should not be construed as limiting. The contents of all references, published patent applications and patents cited throughout the present application are hereby incorporated by reference in their entirety. A number of embodiments of the invention have been described. Nevertheless, it will be understood that various modifications may be made without departing from the spirit and scope of the invention.

### Example 1

### Structure and Sequence of pGA1

pGA1 as illustrated in FIG. 1 and FIG. 2 contains the ColE1 origin of replication, the kanamycin resistance gene for antibiotic selection, the lambda T0 terminator, and a eukaryotic expression cassette including an upstream intron. The ColE1 origin of replication is a 1059 by nucleotide DNA fragment that contains the origin of replication (ori), encodes an RNA primer, and encodes two negative regulators of replication initiation. All enzymatic functions required for replication of the plasmid are provided by the bacterial host. The originally constructed plasmid that contained the ColE1 replicator was pBR322 (Bolivar et al., Gene i: 95-113, 1977; Sutcliffe et al., Cold Spring HarborQuant. Biol. 43:77-90, 1978).

The kanamycin resistance gene is an antibiotic resistance gene for plasmid selection in bacteria. The lambda T0 terminator prevents read through from the kanamycin resistance gene into the vaccine transcription cassette during prokaryotic growth of the plasmid (Scholtissek et al., Nucleic Acids Res. 15:3185, 1987). By preventing read through into the vaccine expression cassette, the terminator helps stabilize plasmid inserts during growth in bacteria.

The eukaryotic expression cassette is comprised of the CMV immediate early (CMVIE) promoter, including intron A (CMV Intron A), and termination sequences from the bovine growth hormone polyadenylation sequence (BGHpA). A synthetic mimic of the leader sequence for tissue plasminogen activator (tP A) is included as an option within the transcription cassette. Cassettes with these elements have proven to be highly effective for expressing foreign genes in eukaryotic cells (Chapman et al., Nucleic Acids Research 12.:3979-3986, 1991). Cloning sites within the transcription cassette include aCla I site upstream of the tP A leader, a Nhe I site for cloning in frame with the tPA leader, and Xmn I, Sma I, Rsr II, Avr II, and Bln I sites for cloning prior to the BGHpA.

The ColE1 replicator, the kanamycin resistance gene and the transcriptional control elements for eukaryotic cells were combined in one plasmid using PCR fragments from the commercial vector pZEr0-2 (Invitrogen, Carlsbad, Calif.) and a eukaryotic expression vector pJW4303 (Lu et al., Vaccine 15:920-923, 1997).

A 1853 bp fragment from pZEr02 from nt 1319 to nt 3178 included the ColE1 origin of replication and the kanamycin resistance gene. A 2040 by fragment from pJW4303 from nt 376 to nt 2416 included the CMVIE promoter with intron A, a synthetic homolog of the tissue plasminogen activator leader (tPA), and the bovine growth hormone polyadenylation site (BGHpA). Fragments were amplified by polymerase chain reaction (PCR) with oligonucleotide primers containing Sal I sites. A ligation product with the transcription cassettes for kanamycin resistance from pZeR02 and the eukaryotic transcription cassette form pJW 4303 in opposite transcriptional orientations, was identified for further development. Nucleotide numbering for this parent of the pGA vectors was started from the first by of the 5′ end of the CMV promoter.

The T0 terminator was introduced into this parent for the pGA vectors by PCR amplification of a 391 by fragment with a BamH 1 restriction endonuclease site at its 5′end and an Xba I restriction endonuclease site at its 3′ end. The initial 355 bp of the fragment were sequences in the BGHpA sequence derived from the pJW4303 transcription cassette, the next 36 bases in a synthetic oligonuclotide introduced the To sequence and the Xba I site. The introduced T0 terminator sequences comprised the sequence: 5′-ATAAAAAACGCCCGGCGGCAACCGAGCGTTCTGAA-3′ (SEQ IDNO: 6).

The T0 terminator containing the BamH l-Xba I fragment was substituted for the homologous fragment without the T0 terminator in the plasmid created from pZEr0-2 and pJW4303. The product was sequenced to verify the T0 orientation, as shown in FIG. 2.

A region in the eukaryotic transcription cassette between nucleotides 1755-1845 contained the last 30 bp of the reading frame for SIV nef. This region was removed from pGA by mutating the sequence at ntl 858 and generating an Avr II restriction endonuclease site. A naturally occurring Avr II site is located at ntl 755. Digestion with Avr II enzyme and then religation with T4 DNA ligase allowed for removal of the SIVsegment of DNA between nucleotides 1755-1845. To facilitate cloning of HIV-1 sequences into pGA vectors, a Cla I site was introduced at by 1645 and an Rsr II site at bp 1743 using site directed mutagenesis. Constructions were verified by sequence analyses.

### Example 2

### Structure and Sequence of pGA2; pGA2-Based Vaccines

pGA2 is schematically illustrated in FIG. 3, and its nucleotide sequence (SEQ IDNO: 2) is shown in FIG. 4. pGA2 is identical to pGA1 (SEQ ID NO: 1) except that the intron A sequence has been deleted from the CMV promoter of pGA2. pGA2 was created from pGA1 by introducing a Cla I site 8 by downstream from the mRNA cap site in the CMV promoter. The Cla I site was introduced using oligonucleotide-directed mutagenesis using the complimentary primers having the sequences: 5′-CCGTCAGATCGCATCGATACGCCATCCACG-3′ (SEQ ID NO:7) and 5′-CGTGGATGGCGTATCGATGCGATCTGACGG-3′ (SEQ ID NO:8). After insertion of the new Cla I site, pGA1 was digested with Cla I to remove the 946 by Cla I fragment from pGA1, and then religated to yield pGA2.

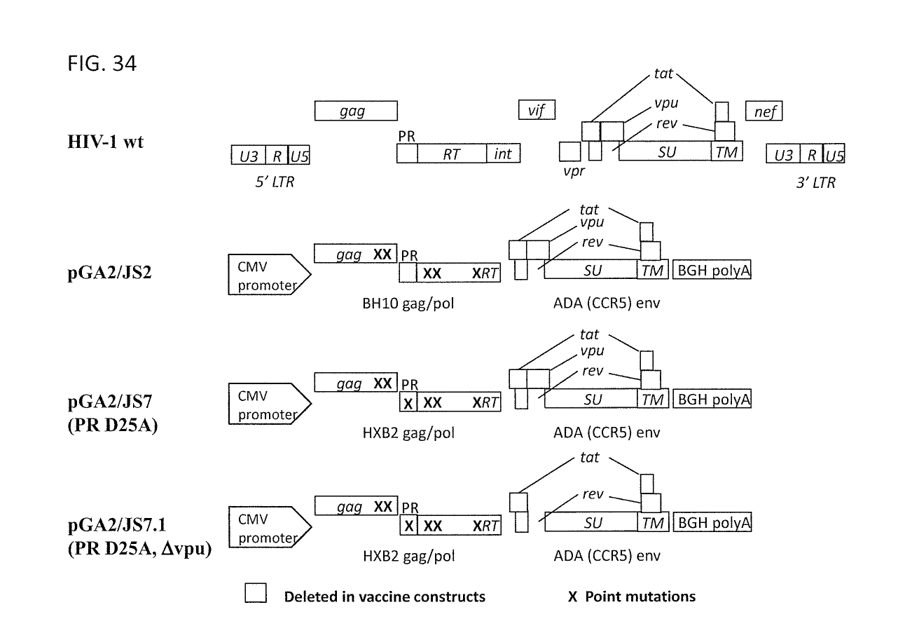

As noted herein, vectors having one or more of the features or characteristics (particularly the oriented termination sequence and a strong promoter) of the plasmids designated pGA1, pGA2, or pGA3 (including, of course, those vectors per se), can be used as the basis for a vaccine. These vectors can be engineered using standard recombinant techniques to include sequences that encode antigens that, when administered to or expressed in a patient, will induce or enhance an immune response that provides the patient with some form of protection against the pathogen from which the antigens were obtained or derived (e.g., protection against infection or protection against disease). As described in this and other Examples, several plasmids have been constructed and used to express antigens. For example, the pGA2/JS2 construct has gone through immunogenicity studies in macaques. Two additional DNA vaccine constructs (pGA2/JS7 and pGA2/JS7.1 (FIG. 34) have been constructed and partially characterized. These constructs may exhibit better immunogenicity and priming efficiency than pGA2/JS2. pGA2/JS7 and pGA2/JS7.1 differ from pGA2/JS2 in several aspects, one of which is that the source of the Gag and Pol genes was changed from HIV-1 BH10 (in pGA2/JS2) to HIV-1 HXB2 (in pGA2/JS7 and pGA2/JS7.1). This change was made in an attempt to obtain a true VLP-forming immunogen rather than aggregates of protein and little virus like particle (VLP) formation seen with pGA2/JS2. With an additional mutation in the viral protease gene (D25A), the pGA2/JS7 and pGA2/JS7.1 constructs both produce VLPs in abundance. Additional point mutations in the vpu gene in pGA2/JS7.1 resulted in a loss of Vpu expression and an increase in Env expression. The increase in Env expression does not compromise Gag expression. The pGA2/JS7 construct is currently in a macaque immunogenicity study against the original pGA2/JS2 to determine if there is an increase in priming efficiency over that seen with pGA2/JS2.

Analogous changes can be made in any vaccine insert that includes gag, pol; any vaccine insert that encodes a viral protease; or any vaccine insert that includes a vpu gene. Moreover, these changes can be made in vaccine inserts that are placed in any of the plasmid or live-vectored vaccines described herein (i.e., in any plamid having one or more of the features or characteristics of the pGA vectors, the pGA vectors themselves, or the vaccinia vectors that may be used alone or in conjunction with (e.g., to boost) a DNA-primed patient).

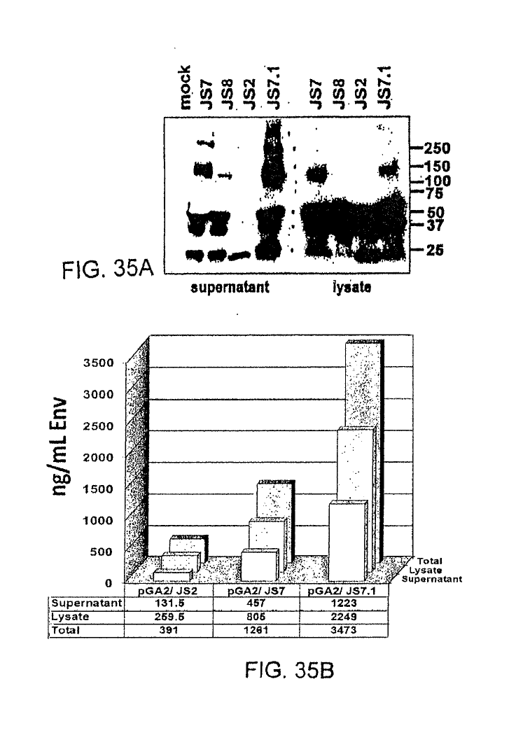

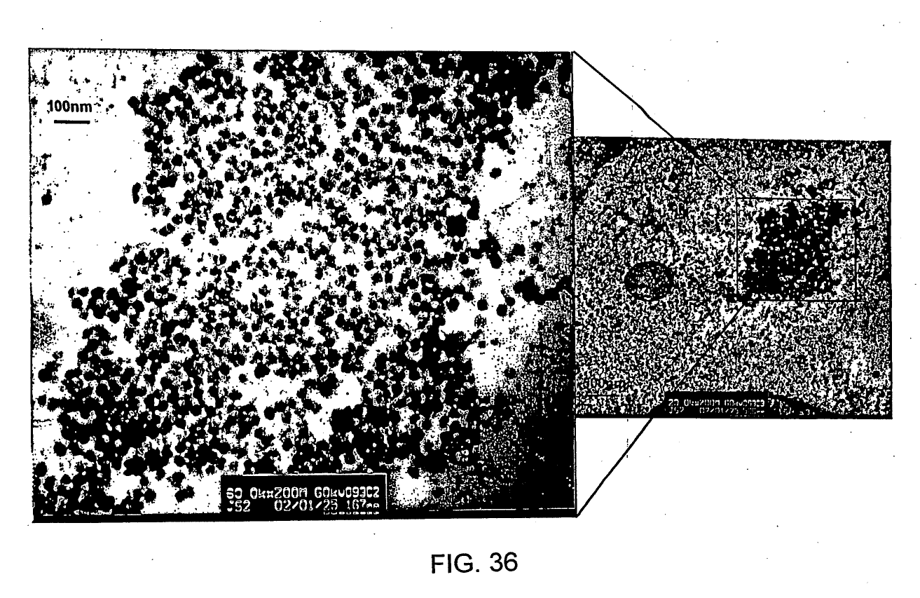

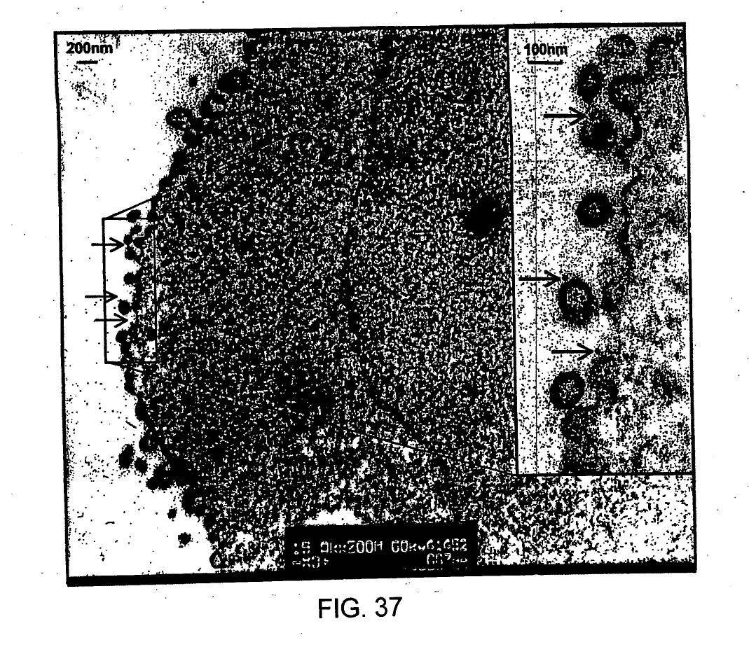

Further characterization of the JS7 and JS7.1 inserts, including evaluations of expression and examination of VLP formation (by electron microscopy) has been done, and the results are shown in FIGS. 35A, 35B, 36, and 37 (see the legends above).

### Example 3

### Structure and Sequence of pGA3

pGA3 is schematically illustrated in FIG. 5, and its nucleotide sequence (SEQ IDNO: 3) is shown in FIG. 6. pGA3 is identical to pGA1 except that a Hind III site has been introduced in place of the Cla I site at nucleotide 1645 of pGA1, and a BamH I site has been introduced in place of the Rsr II site at nucleotide 1743 of pGA1. Accordingly, the pGA3 vector is an embodiment of the invention; as are pGA1 and pGA2; as are plasmid vectors having one or more of the features or characteristics of a pGA plasmid (see the detailed description), but different restriction endonuclease sites in the multi-cloning site (e.g., the invention encompasses plasmids that are otherwise substantially similar to pGA1, pGA2, or pGA3 but that have more, less, or different restriction endonuclease sites in their multi-cloning site).

### Example 4

### Comparative Expression and Immunogenicity of pGA3 and pJW4303

To determine the efficacy of the pGA plasmids as vaccine vectors, a pGA plasmid was compared to the previously described vaccine vector pJW 4303. Any plasmid can be assessed for use as a DNA vaccine, just as the pGA3 plasmid is assessed here. Plasmids that have substantially the same sequence as the pGA vectors described herein are within the scope of the invention so long as they are immunogenic enough to induce or enhance a therapeutically beneficial response in a patient (a plasmid can have substantially the same sequence as a pGA vector even if one or more of the component parts of the plasmid, such as the marker gene or antibiotic-resistance gene, has been deleted).

The pJW 4303 plasmid has been used for DNA vaccinations in mice, rabbits, andrhesus macaques (Robinson et al., Nature Medicine 5:526, 1999; Robinson et al., TheScientific Future of DNA for Immunization, American Academy of Microbiology, May 31-Jun. 2, 1996, 1997; Pertmer et al., Vaccine 13:1427-1430, 1995; Feltquate et al., J. Immunol. 158:2278-2284, 1997; Torres et al., Vaccine 18:805-814, 1999). Comparisons were made between pGA3 with a vaccine insert encoding the normal, plasma-membrane form of the NPR/8/34 (H1N1) influenza virus hemagglutinin (pGA3/H1) and pJW4303 encoding the same fragment (pJW4303/H1). Both pGA3 and pJW 4303 contain the CMV-Intron A upstream of influenza H sequences.

The pGA3/HI and pJW 4303/HI vaccine plasmids expressed similar levels of H1 in eukaryotic cells, as summarized below:

TABLE 1
In Vitro Expression Levels of HA plasmids.

| | Relative HA Units | |
|---|---|---|
| Plasmids | Supernatant | Cell Lysate |
| p GA3/Hl | 0.1 ± 0.1 | 5.7 ± 0.6 |
| pGA vector | 0.0 ± 0.0 | 0.2 ± 0.1 |
| pJX4303/Hl | 0.3 ± 0.05 | 4.8 ± 0.5 |
| pJW4303 | 0.0 ± 0.0 | 0.1 ± 0.1 |

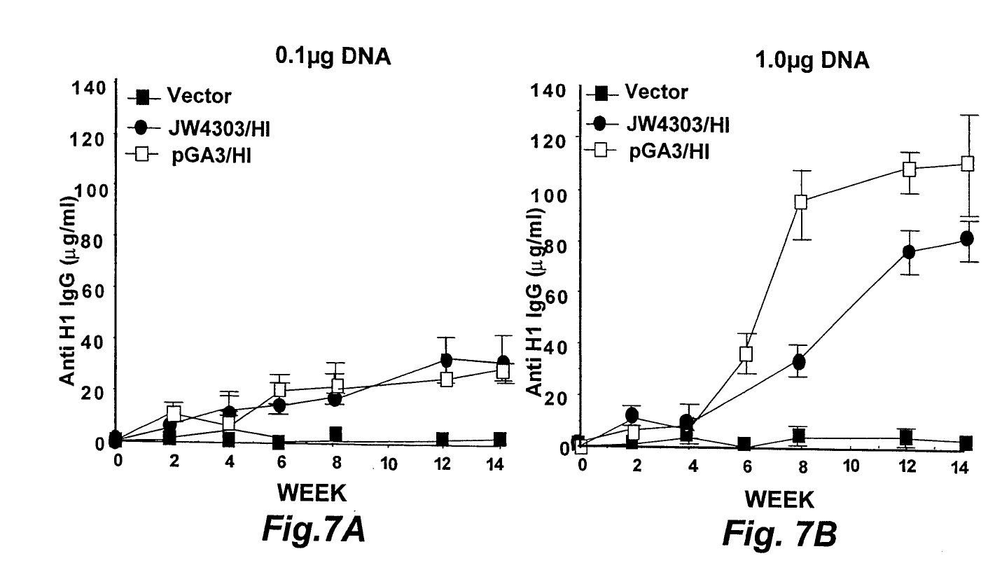

Human embryonic kidney 293T cells were transiently transfected with 2 μg of plasmid and the supernatants and cell lysates were assayed for H1 using an antigen capture ELISA. The capture antibody was a polyclonal rabbit serum against H1, and the detection antibody was polyclonal mouse serum against H1. pGA3/H1 expressed slightly more H1 than pJW4303/H1 (5.8 HA units as opposed to 5.1 H1 units (see Table 1)). As expected, 90% of the H1 antigen was in the cell lysate. A comparative immunization study using pGA3/H1 and pJW 4303/H1 demonstrated comparable or better immunogenicity for pGA3/H1 than pJW4303/H1 (FIG. 7). Immunogenicity was assessed in BALB/c mice. In this example, mice were vaccinated with DNA coated gold particles delivered biolistically (i.e., via gene gun). Mice were primed and boosted with a low dose (0.1 μg) or a high dose (1.0 μg) of the plasmid DNAs. The booster immunization was given at 4 weeks after the priming immunization. The amount of anti-H1 IgG raised in response to immunizations was as high or higher following immunization with pGA3/H1 than following immunization with pJW4303/H1 (FIG. 7). Thus, the pGA vector proved to be as effective, or more effective, than the pJW 4303 vector at raising immune responses.

### Example 5

### Immunodeficiency Virus Vaccine Inserts in pGA Vectors

Immunodeficiency virus vaccine inserts expressing VLPs were developed in pGA1 and pGA2. The VLP insert was designed with clade B HIV-I sequences so that it would match HIV-I sequences that are endemic in the United States. Within clade B, different isolates exhibit clustal diversity, with each isolate having overall similar diversity from the consensus sequence for the clade (Subbarao et al., AIDS 11(Suppl A): S 13-23, 1996). Thus, any clade B isolate can be used as a representative sequence for other clade B isolates. Accordingly, the compositions of the invention can be made with, and the methods described herein can be practiced with, natural variants of genes or nucleic acid molecules that result from recombination events, alternative splicing, or mutations.

HIV-1 isolates use different chemokine receptors as co-receptors. The vast majority of viruses that are undergoing transmission use the CCR-5 co-receptor (Berger, AIDS 11(Suppl A):53-16, 1997). Therefore, the vaccine insert was designed to have a CCR-5-using Env. Of course, Envs that function through any other receptor can be made and used as well (alone or in combination).

The expression of VLPs with a CCR-5-tropic (R5) HIV-1 Env by a HIV-1 DNA vaccine also has the advantage of supporting Env-mediated entry of particles into professional antigen presenting cells (APCs), such as dendritic cells and macrophages. Both dendritic cells and macrophages express the CD4 receptor and the CCR-5 coreceptor used by a CCR-5-tropic (R5) HIV-1 Env. By using an R5-Env in the vaccine, the VLP expressed in a transfected non-professional APC (for example keratinocyte or muscle cells) can gain entry into the cytoplasm of an APC by Env-mediated entry. Following entry into the cytoplasm of the APC, the VLP will be available for processing and presentation by Class I histocompatibility antigens. DNA-based immunizations rely on professional APCs for antigen presentation (Corr et al., J. Exp. Med. 184:1555-1560, 1996; Fu, et al., Mal. Med. J.:362-371, 1997; Iwasaki et al., 1997). Much of DNA-based immunization is accomplished by direct transfection of professional APC (Condon et al., 1996; Porgador et al., J Exp. Med. 188:1075-1082, 1998).

Transfected muscle cells or keratinocytes serve as factories of antigen but do not directly raise an immune response (Torres et al., J. Immunol. 158:4529-4532, 1997). By using an expressed antigen that is assembled and released from transfected keratinocytes or muscle cells and then actively enters professional APC, the efficiency of the immunization may be increased.

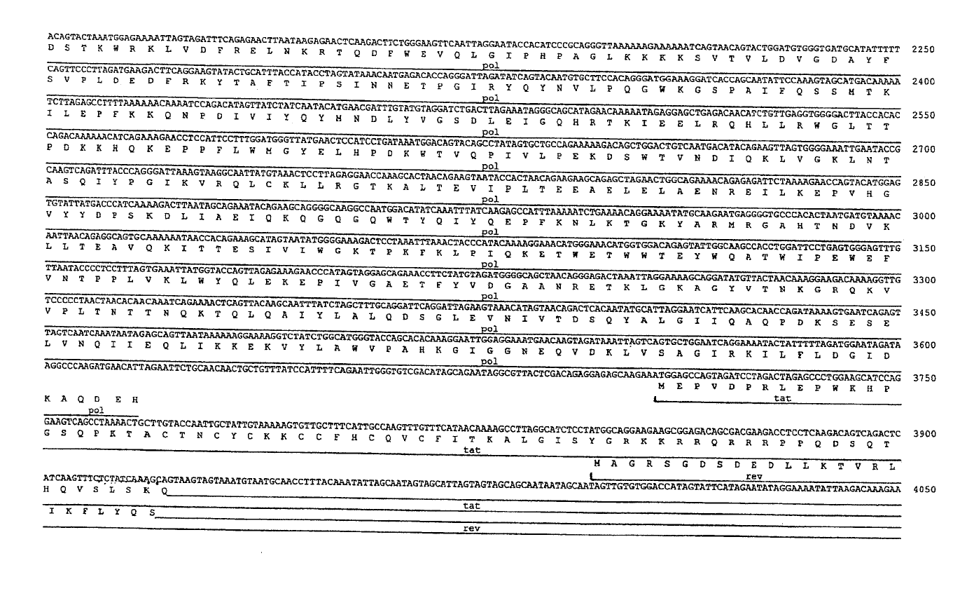

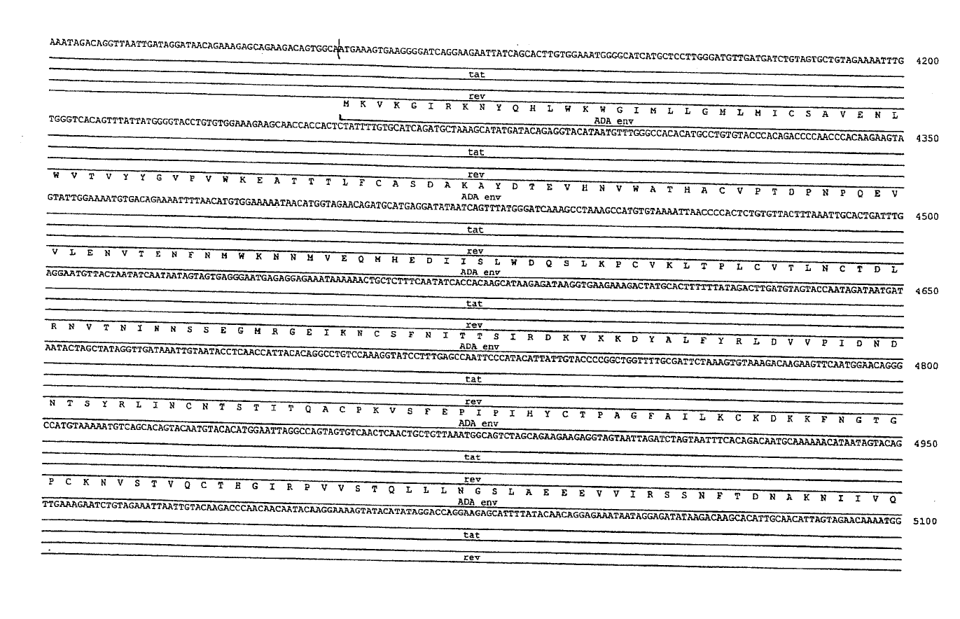

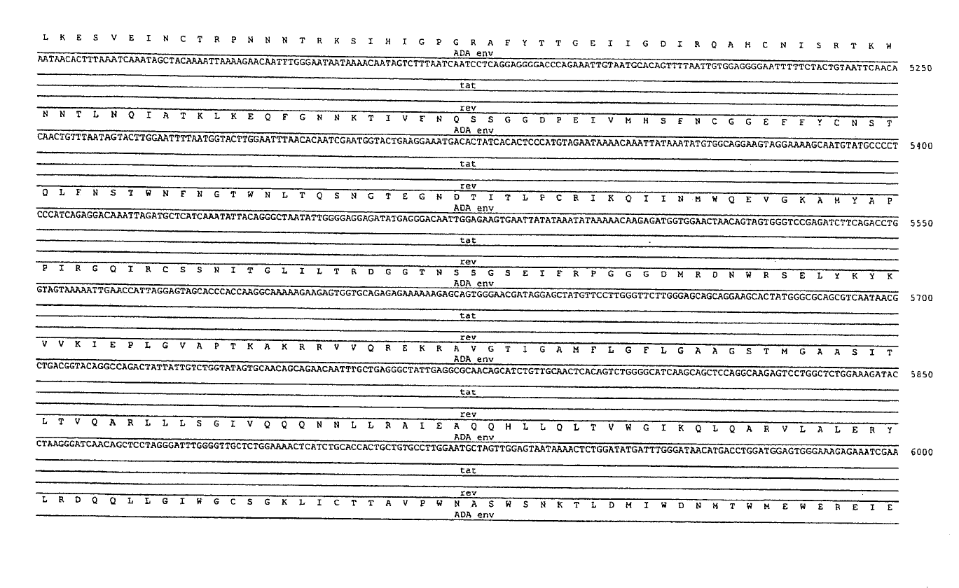

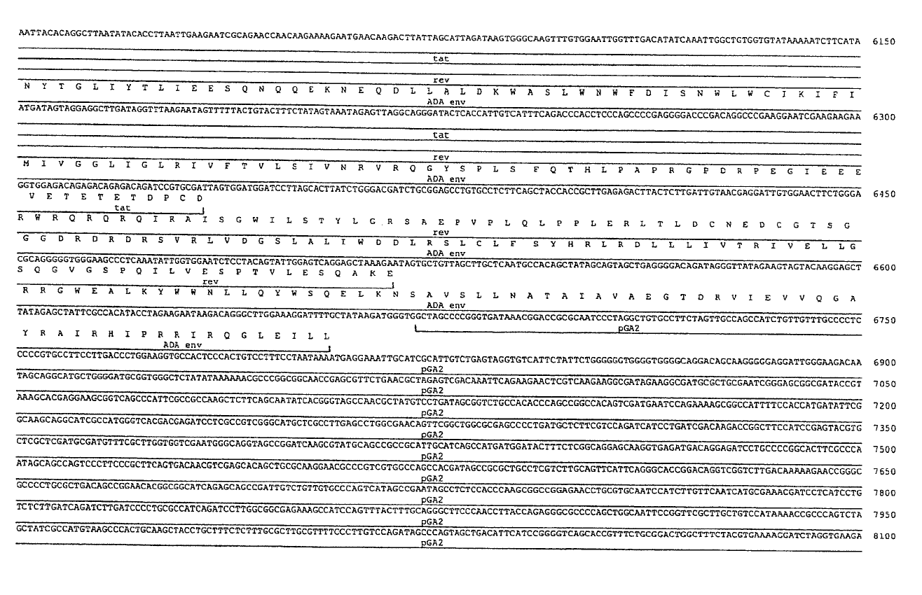

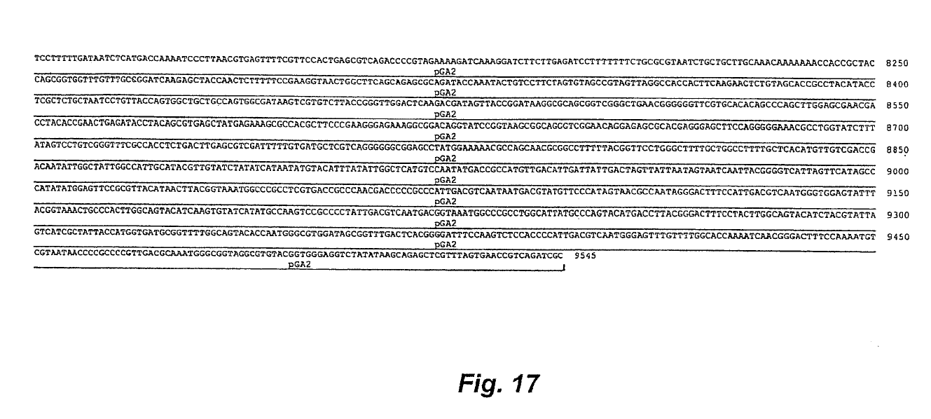

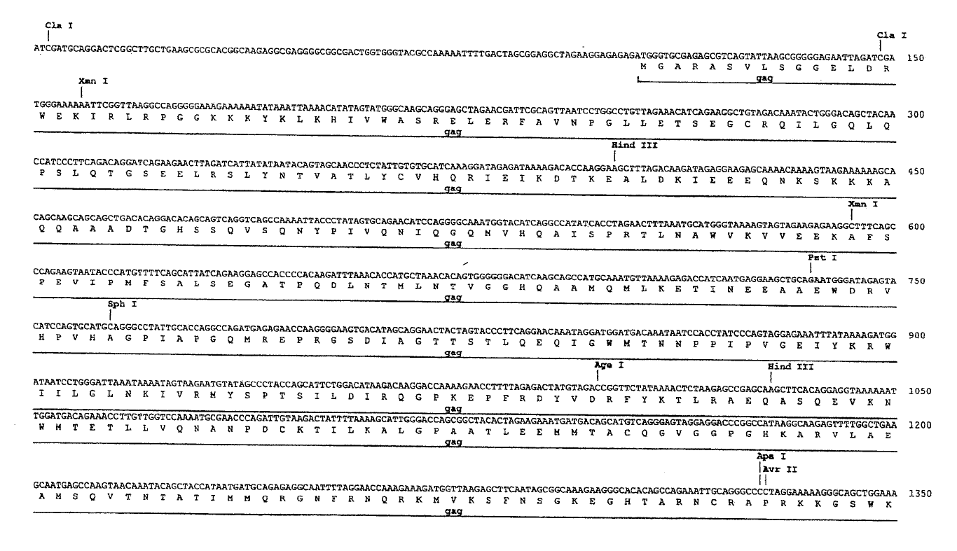

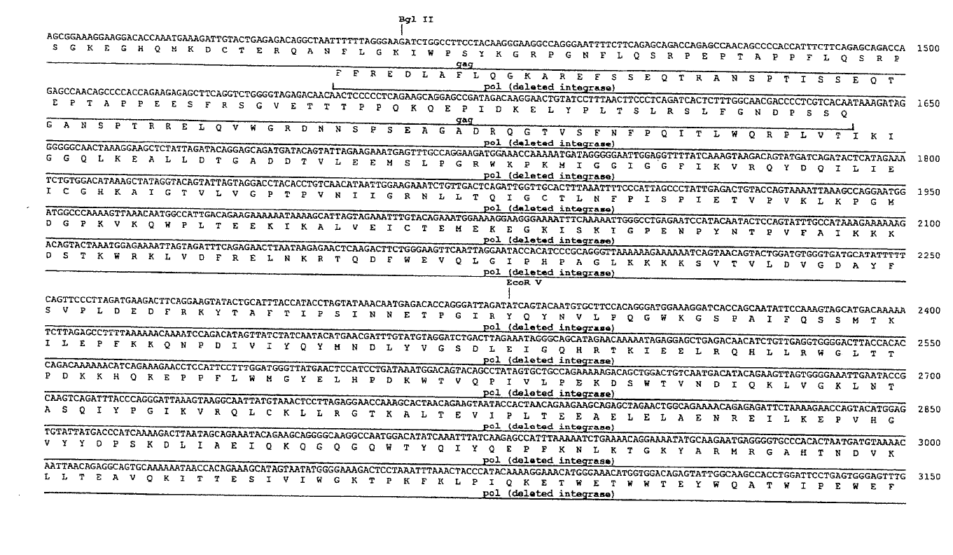

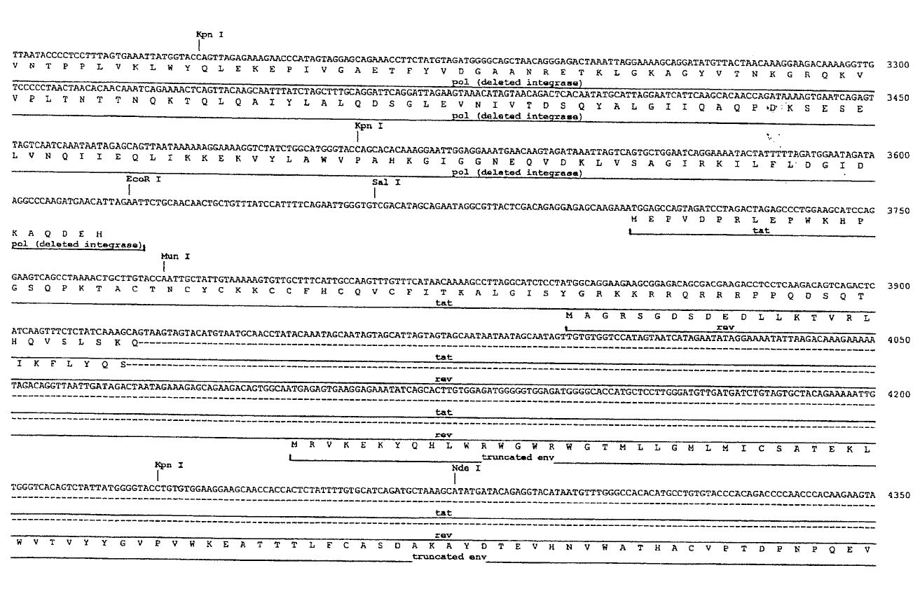

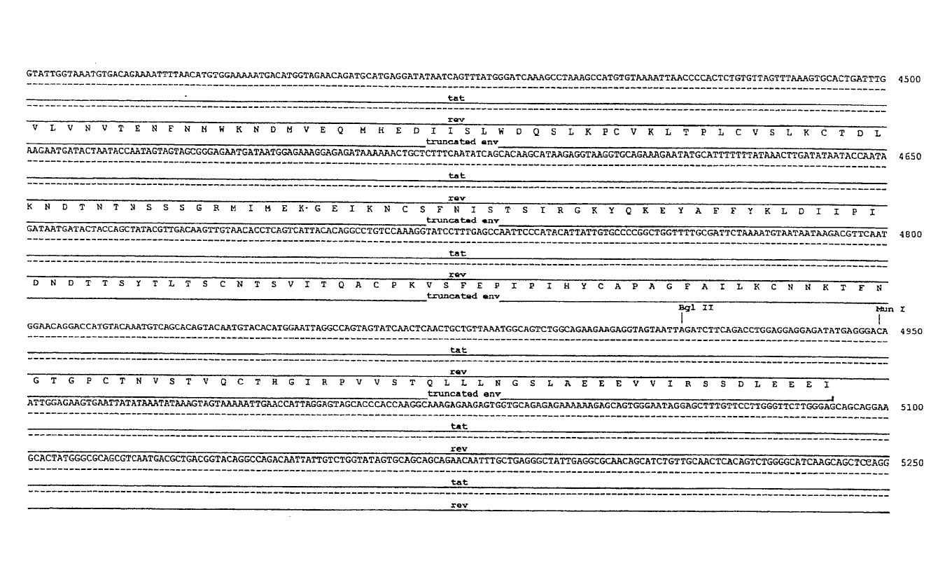

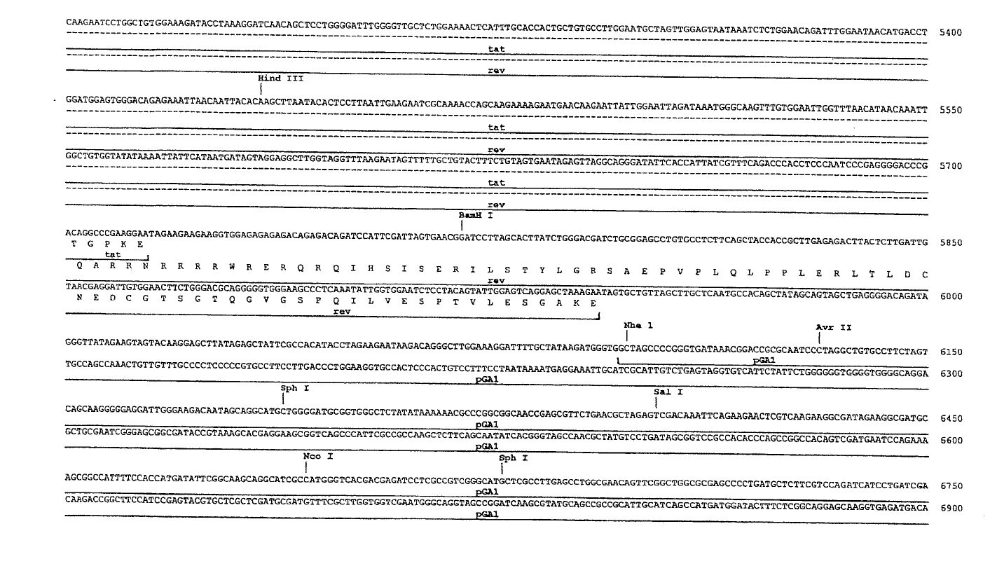

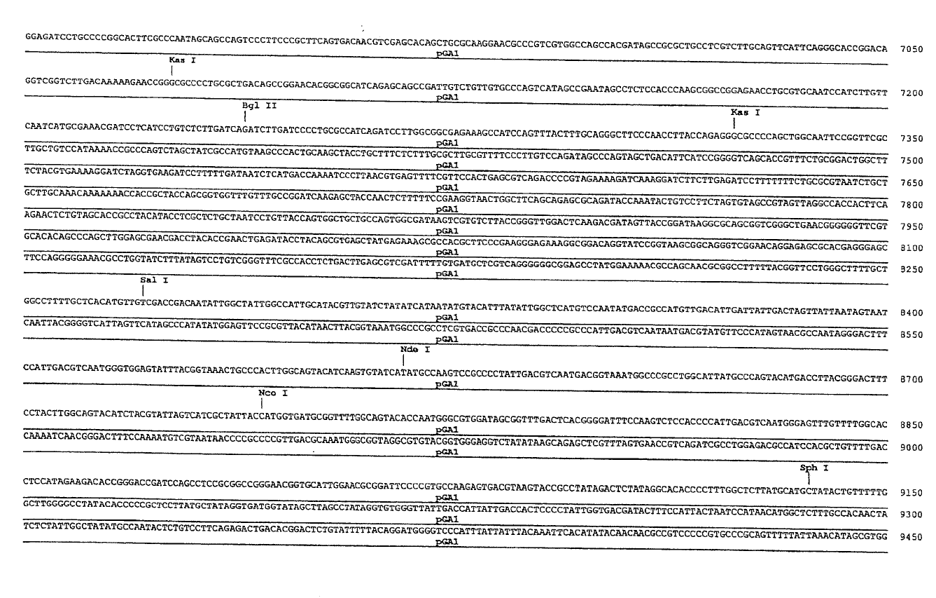

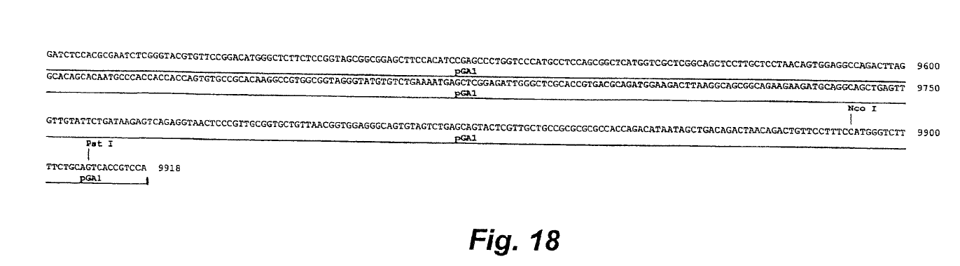

Goals in the construction of pGA2/JS2 included (i) achieving a CCR-5-using clade B VLP with high expression, (ii) producing a non-infectious VLP; and (iii) minimizing the size of the vaccine plasmid. Following the construction of the CCR-5-using VLP (pGA2/JS2), a derivative of JS2 was prepared that expresses an Env defective VLP. This plasmid insert was designated JS5. Non-Env containing VLPs may advantageous because one can monitor vaccinated populations for infection by seroconversion to Env. Deletion of Env sequences also reduces the size of the vaccine plasmid. The DNA sequence of pGA2/JS2 (SEQ ID NO: 4) is shown in FIG. 17. The DNA sequence of pGA1/JS5 (SEQ ID NO: 5) is shown in FIG. 18.

To achieve a VLP plasmid with high expression, candidate vaccines were constructed from seven different HIV-1 sequences, as shown in the following table.

TABLE 2
Comparison of candidate vaccine inserts

| | | Ability to | | | |
|---|---|---|---|---|---|
| Plasmid | | grow | Expression | Expression | |
| designation | Sequences tested | plasmid | of Gag | of Env | Comment |
| BH10-VLP | BH10 | good | Good | good | X4Env |
| 6A-VLP | 6A env in | poor | not tested | not tested | |
| | BH10-VLP | | | | |
| BAL-VLP | BALenv in | good | Poor | Poor | |
| | BH10-VLP | | | | |
| ADA-VLP | ADAenv in | good | Good | good | chosen for vaccine, |
| | BH10-VLP | | | | renamed pGA1/JS1 |
| CDC-A-VLP | CDC-A env | good | Good | Poor | |
| | in BH10-VLP | | | | |
| CDC-B-VLP | CDC-B-env | good | Good | good | not as favorable |
| | in BH10-VLP | | | | expression as ADA |
| CDC-C-VLP | CDC-C env | good | Good | good | not as favorable |
| | In BH1O-VLP | | | | expression as ADA35 |

An initial construct, pBH10-VLP, was prepared from IIIB sequences that are stable in bacteria and have high expression in eukaryotic cells. The HIV-1-BH10 sequences were obtained from the NIH-sponsored AIDS Repository (catalog #90). The parental pHIV-1-BH10 was used as the template for PCR reactions to construct pBH10VLP.

Primers were designed to yield a Gag-Rt PCR product (5′ PCR product) encompassing (from 5′ to 3′) 105 by of the 5′ untranslated leader sequence and gag and pol sequences from the start codon for Gag to the end of the RT coding sequence. The oligonucleotide primers introduced a Cla I site at the 5′ end of the PCR product and EcoR I and Nhe I sites at the 3′ end of the PCR product. Sense primer 1(5′-GAGCTCTATCGATGCAGGACTCGGCTTGC-3′ (SEQ ID NO: 9)) and antisense primer 2 (5′-GGCAGGTTTTAATCGCTAGCCTATGCTCTCC-3′ (SEQ ID NO: 10)) were used to amplify the 5′ PCR product.

The PCR product for the env region of HIV-1 (3′ PCR product) encompassed the vpu, tat, rev, and env sequences and the splice acceptor sites necessary for proper processing and expression of their respective mRNAs. An EcoR I site was introduced at the 5′ end of this product and Nhe I and Rsr II sites were introduced into the 3′ end. Sense primer 3 (5′-GGGCAGGAGTGCTAGCC-3′ (SEQ ID NO: 11)) and antisense primer 4 (5′-CCACACTACTTTCGGACCGCTAGCCACCC-3′ (SEQ ID NO: 12)) were used to amplify the 3 ′ PCR product.

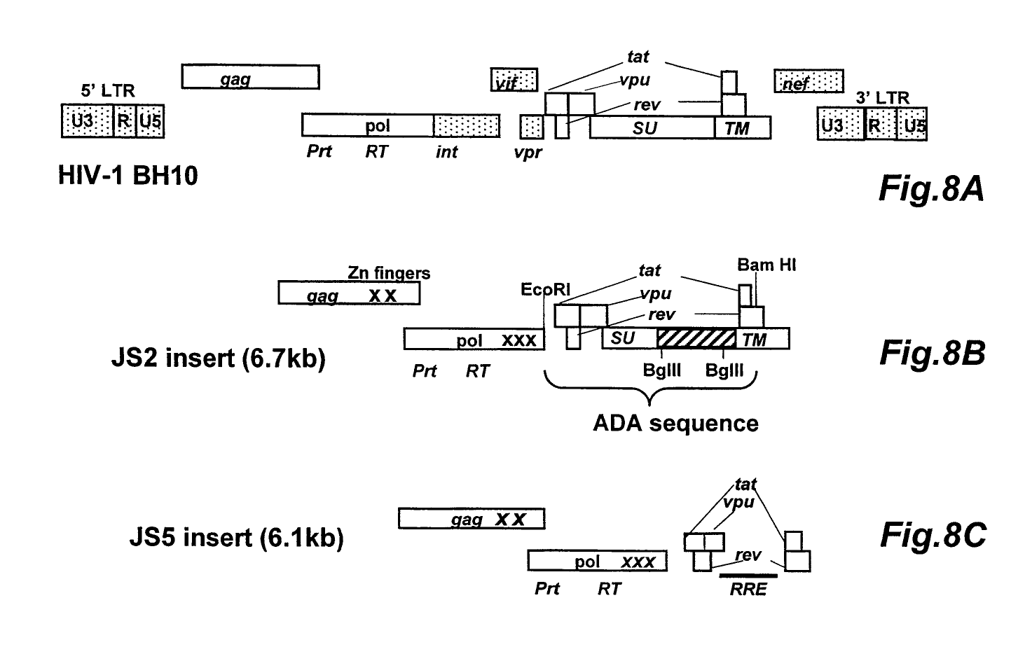

The 5′ PCR product was cloned into pGA1 at the Cla I and Nhe I sites and the identity of the construct confirmed by sequencing. The 3′ PCR product was then inserted into the 5′ clone at the EcoR I and Nhe I sites to yield pBH1O-VLP. The construction of this VLP resulted in proviral sequences that lacked LTRs, integrase, vif, and vpr sequences (FIG. 8).

Because the BH10-VLP had an X4 Env, rather than an R5 Env, sequences encoding six different R5 Envs were substituted for env sequence in BH1O-VLP. The substitution was made by cloning EcoR I to BamH I fragments encompassing tat, rev, vpu and env coding sequences from different viral genomes into pBH10-VLP. The resulting env and rev sequences were chimeras for the substituted sequences and HIV-1-BH10 sequences (for example, see Fig. SB). In the case of the HIV-1-ADA envelope, a 36BamH I site was introduced into the HIV-1-ADA sequence to facilitate substituting an EcoR I to BamH I fragment for the EcoR I to BamH I region of the BH1O-VLP (FIG. 8). The results of these constructions are summarized in Table 1. Of the six sequences tested, one, the 6A-VLP gave poor plasmid growth in transformed bacteria (plasmids having any given or desired insert can be similarly assessed). The plasmid 6A-VLP was not developed further (Table 2).

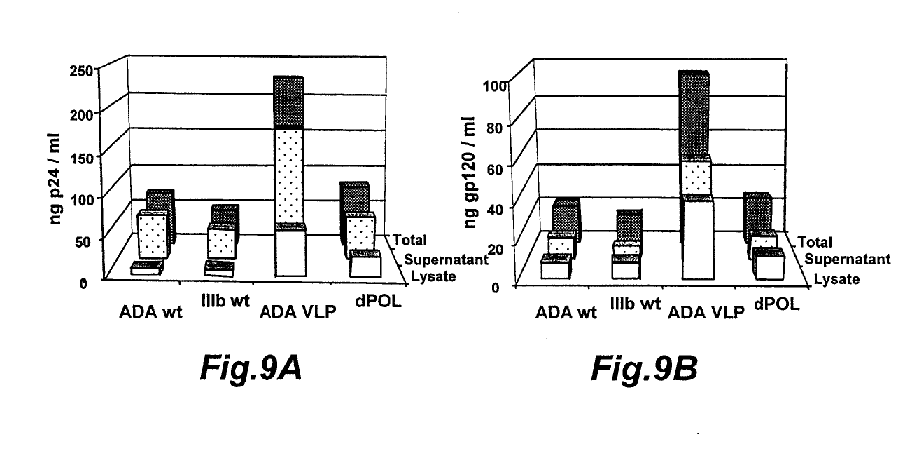

Although most plasmids grew well in bacteria, the ADA-VLP construct produced the best expression of a VLP (Table 2). In transient transfections in 293T cells, the expression of the ADA-VLP was higher than that of wt proviruses for HIV-I-ADA or HIV-1-IIIB (FIGS. 9A and 9B). Expression was also higher than for a previous VLP vaccine (dpo1) (Richmond et al., J Viral. 72:9092-9100, 1998) that had successfully primed cytotoxic T cell responses in rhesus macaques (Kent et al., J Viral. 72:10180-10188, 1998).

### Example 6

### Safety Mutations

Once the ADA-VLP had been identified as a favorable candidate for further vaccine development, this plasmid was mutated to increase its safety for use in humans. Further mutations disabled the Zinc fingers in NC that are active in the encapsidation of viral RNA, and added point mutations to inactivate the viral reverse transcriptase and the viral protease, as shown in FIGS. 8B and 8C. Table 3 summarizes the location of the safety point mutations. One or more of these mutations can be included in vaccine inserts that, like JS2 and JS5, include gag, pol (i.e., any vaccine insert in any vector that encodes Gag, Pol). Alternatively, a protein can be inactivated by deleting all or part of the gene sequence that encodes it, rather than by introducing point mutations.

TABLE 3
Locations of safety point mutations in pGA/JS2 and
pGA/JS5 introduced to inhibit viral RNA packaging and
abolish reverse transcriptase activity in vaccine constructs

| | | | AMINO ACID | |
|---|---|---|---|---|
| GENE | REGION | FUNCTION | CHANGE¹ | LOCATION² |
| Gag | Zn finger | Viral RNA | C392S | 1285/1287 |
| | | packaging | | |
| Gag | Zn finger | Viral RNA | C392S | 1294/1296 |
| | | packaging | | |
| Gag | Zn finger | Viral RNA | C413S | 1348/1350 |
| | | packaging | | |
| Gag | Zn finger | Viral RNA | C416S | 1357/1359 |
| | | packaging | | |
| Pol | RT | Polymerase | D185N | 2460/2462 |
| | | activity | | |
| Pol | RT | Strand transfer | W266T | 2703/2704/2705 |
| Pol | RNAseH | RNAse activity | E478Q | 3339 |

¹Amino acid number corresponds to individual genes in HIV-1-BH10 sequence,
²Nucleotide number in wt HIV-1-BH 10 sequence.

The mutations were made using a site directed mutagenesis kit (Stratagene) following themanufacturer's protocol. All mutations were confirmed by sequencing. Primer pairs used for the mutagenesis were:

(A)
C15S ZN1
(SEQ ID NO: 13)
5′-GGTTAAGAGCTTCAATAGCGGCAAAGAAGGGC-3′
C15S ZN2
(SEQ ID NO: 14)
5′-GCCCTTCTTTGCCGCTATTGAAGCTCTTAACC-3′
(B)
C36S ZN3
(SEQ ID NO: 15)
5′-GGGCAGCTGGAAAAGCGGAAAGGAAGG-3′
C36S ZN4
(SEQ ID NO: 16)
5′-CCTTCCTTTCCGCTTTTCCAGCTGCCC-3′
(C)
D185N RT1
(SEQ ID NO: 17)
5′-CCAGACATAGTTATCTATCAATACATGAACGATTTGTATGTAGG-3′
D185NRT2
(SEQ ID NO: 18)
5′-CCTACATACAAATCGTTCATGTATTGATAGATAACTATGTCTGG-3′
(D)
W266T RT3
(SEQ ID NO: 19)
5′-GGGGAAATTGAATACCGCAAGTCAGATTTACCC-3′
W266TRT4
(SEQ ID NO: 20)
5′ GGGTAAATCTGACTTGCGGTATTCAATTTCCCC-3′
(E)
E478Q RT5
(SEQ ID NO: 21)
5′-CCCTAACTAACACAACAAATCAGAAAACTCAGTTACAAGC-3′
E478QRT6
(SEQ ID NO: 22)
5′-GCTTGT AACTGAGTTTTCTGATTTGTTGTGTTAGTTAGGG-3′
(F)
D25A Prt1
(SEQ ID NO: 23)
5′-GGCAACTAAAGGAAGCTCTATTAGCCACAGGAGC-3′
D25Aprt2
(SEQ ID NO: 24)
5′-GCTCCTGTGGCTAATAGAGCTTCCTTTAGTTGCC-3′

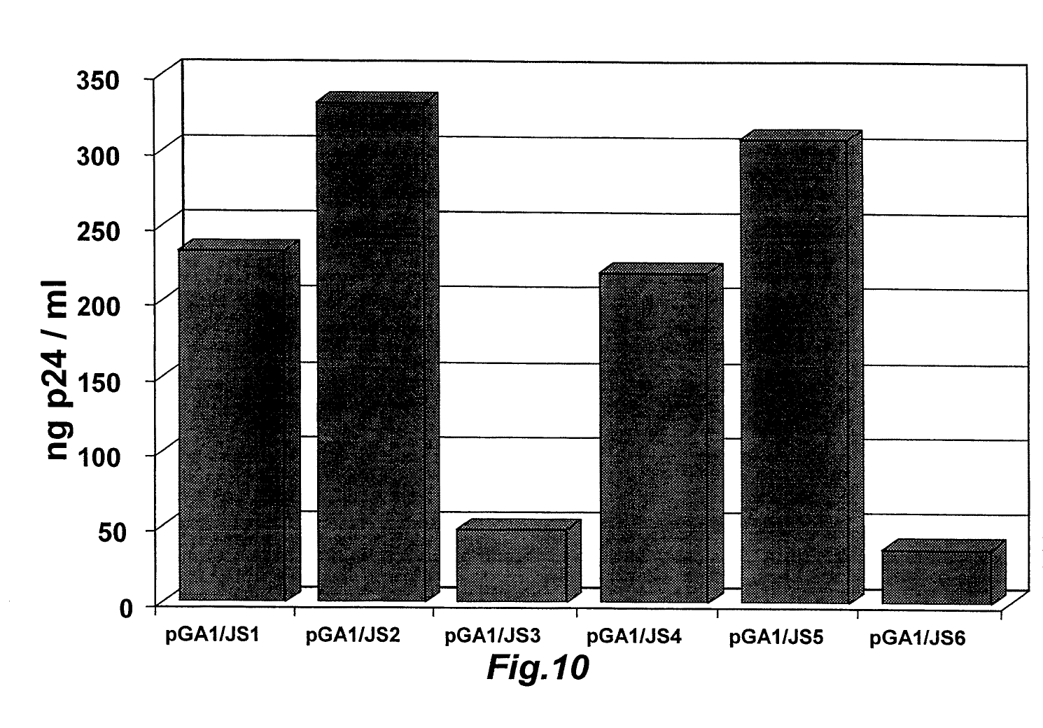

The ADA-VLP with the zinc finger and RT mutations was found to express Gag and Env more effectively than the VLP plasmid without the mutations (FIG. 10). The mutation that inactivated the protease gene markedly reduced VLP expression and was not included in the further development of the vaccine plasmid. The ADA-VLP without mutations was designated JS 1 and the ADA-VLP with mutations was designated JS2.

### Example 7

### Construction of the JSS Vaccine Insert

The JS5 insert, which expresses Gag, RT, Tat, and Rev, was constructed from JS2 by deleting a Bgl II fragment from the HIV-I-ADA Env (Fig. SC). This deletion removed sequences from nt 4906-5486 of the pGA2/JS2 sequence and results in a premature stop codon in the env gene, leading to 269 out of the 854 amino acids of Env being expressed while leaving the tat, rev, and vpu coding regions, the RRE, and the splice acceptor sites intact. The DNA sequence of pGA1/JS5 is shown in FIG. 18 (SEQ ID NO: 5).

### Example 8

### Minimizing the Size of Plasmids that Include the JS2 and JSS Inserts

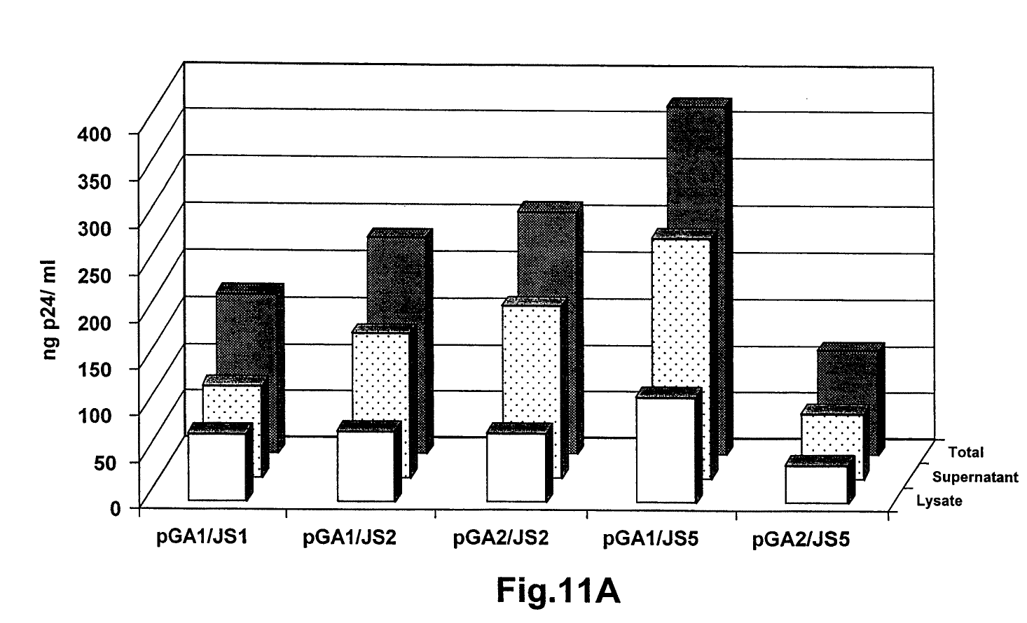

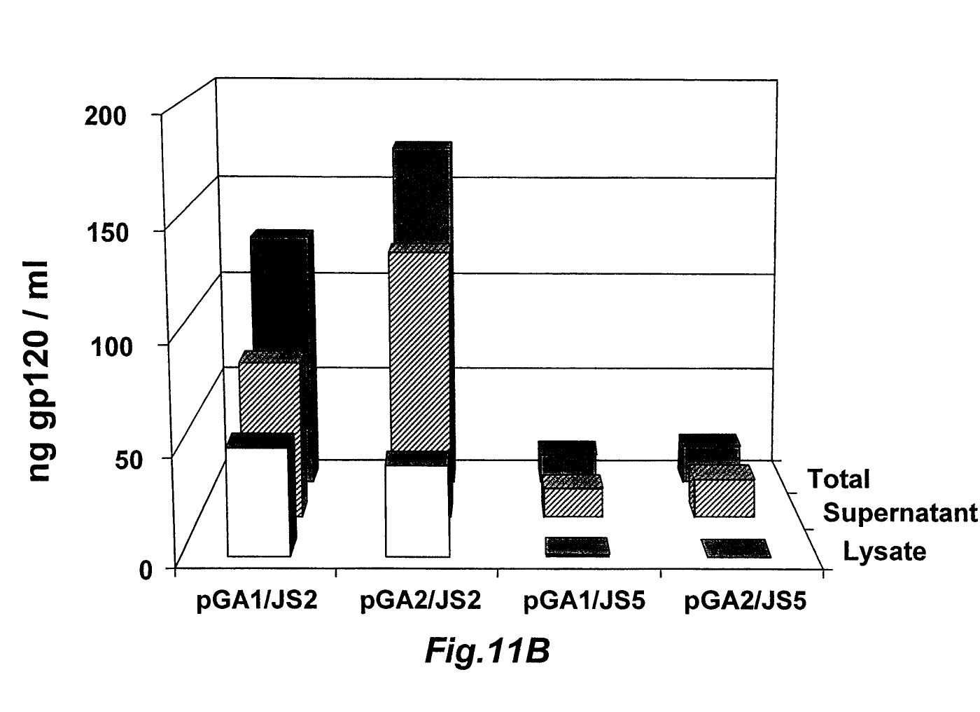

The JS2 and JSS vaccine inserts were constructed in pGA1, a vector that contained the intron A of the CMV intermediate early promoter upstream of the vaccine insert. To determine whether this intron was necessary for high levels of vaccine expression, pGA2 vectors lacking intron A were constructed expressing the JS2 and JS5 vaccine inserts. In expression tests, pGA2 proved to have as good an expression pattern as pGA1 for JS2 (FIGS. 11A and 11B). In contrast, JS5 was expressed much more effectively by pGA1 than pGA2 (FIG. 11A). The absence of intron A resulted in 2-3-fold lower levels of expression of the JS5 insert than in the presence of intron A (FIG. 11A).

### Example 9

### The Efficacy of Safety Mutations in the Vaccine Inserts JS2 and JSS

The three point mutations in RT (see Table 3), completely abolished detectable levels of RT activity for JS2 and JS5. A highly sensitive reverse transcriptase assay was used in which the product of reverse transcription was amplified by PCR (Yamamoto et al., J Viral. Methods 61: 135-143, 1996). This assay can detect reverse transcriptase in as few as 10 viral particles. Reverse transcriptase assays were conducted on the culture supematants of transiently transfected cells. Reverse transcriptase activity was readily detected for as few as 10 particles (4×10⁻³ pg of p24) in the JS1 vaccine, but could not bedetected for the JS2 or JS5 inserts.

The deletions and zinc finger mutations in the JS2 and JS5 vaccine inserts (see Table 3) reduced the levels of viral RNA in particles by at least 1000-fold. Particles pelleted from the supematants of transiently transfected cells were tested for the efficiency of the packaging of viral RNA. The VLPs were treated with DNase, RNA was extracted, and the amount of RNA was standardized by p24 levels before RT-PCR. The RT-PCR reaction was followed by nested PCR using primers specific for viral sequences. End point dilution of the VLP RNA was compared to the signal obtained from RNA packaged in wt HN-1 Bal virus. Packaging for both JS2 and JS5 was restricted by the deletions in the plasmid by 500-1000-fold (see Table 4).

TABLE 4
Packaging of viral RNA is reduced
in pGA1/JS2 and pGA1/JS5 VLPs

| Vaccine | Deletions/ | Copies vRNA relative |
|---|---|---|
| Construct | Mutations | to wt HN-1 bal |
| HIV-1 bal | Wt | 1 |
| pGA1JS1 VLP | Deleted LTRs, int, | .002 |
| | vif, vpr, nef | |
| pGA1/JS2 VLP | Deleted: LTRs, int, | .0001 |
| | vif, vpr, nef | |
| | Mutations in Zn | |
| | fingers and RT | |
| pGA1/JS4 VLP | Deleted LTRs, int, | .001 |
| | vif, vpr, nef | |
| pGA1/JS5 VLP | Deleted: LTRs, int, | .001 |
| | vif, vpr, nef, env; | |
| | Mutations in Zn | |
| | fingers and RT | |

The zinc finger mutations decreased the efficiency of packaging for the JS2 particles a further 20-fold, but did not further affect the efficiency of packaging for the JS5 particles. This pattern of packaging was reproducible for particles produced in independent transfections.

### Example 10

### Western Blot Analyses of Protein Expression

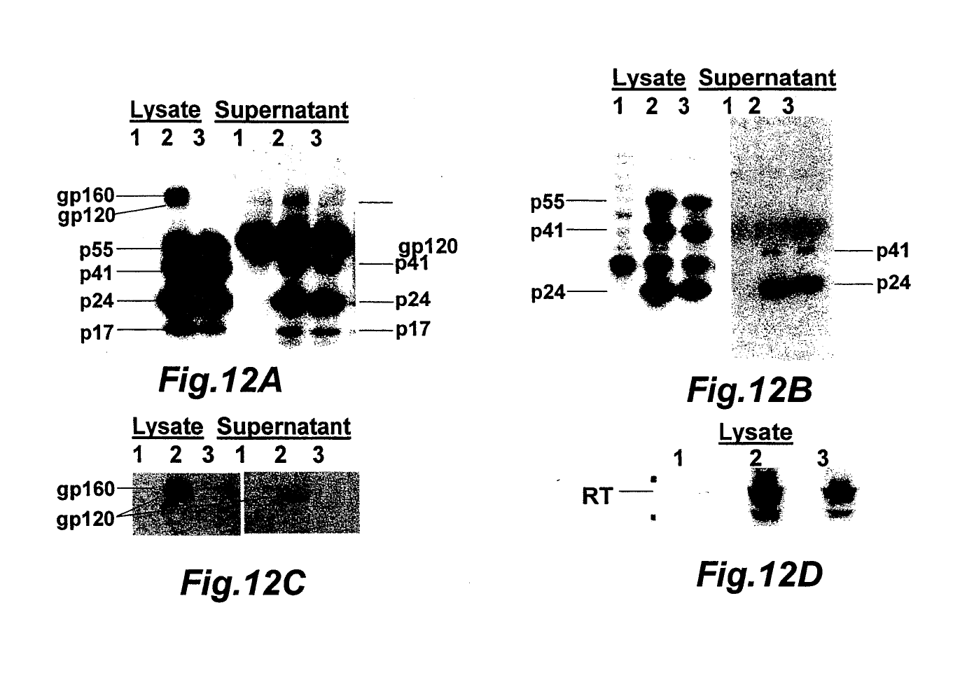

Western blot analyses revealed the expected patterns of expression of pGA2/JS2 and pGA1/JS5 (FIGS. 12A-D). Both immature and mature proteins were observed in cell lysates (FIG. 12A), whereas only the mature forms of Gag and Env were found in the VLP-containing lysates (FIGS. 12B and 12C, respectively). Reverse transcriptase was readily detected in cell lysates (FIG. 12D).

### Example 11

### pGA2/89.6 SHIV Vector Construction

Initial immunogenicity trials have been conducted with a SHIV-expressing VLP rather than the HIV-1-expressing vaccine plasmids. SHIV s are hybrids of simian and human immunodeficiency virus sequences that grow well in macaques (Li et al., J. of AIDS 5:639-646, 1992). By using a SHIV, vaccines that are partially of HIV-1 origin can be tested for efficacy in macaque models.

pGA2/89.6 (also designated pGA2/M2) expresses sequences from SHIV-89.6 (Reimann et al., J. Viral. 70:3198-3206, 1996; Reimann et al., J. Viral. 70:6922-6928, 1996). The 89.6 Env represents a patient isolate (Collman et al., J. Viral. 66:7517-7521, 1992). The SHIV-89.6 virus is available as a highly pathogenic challenge stock, designated SHIV-89.6P (Reimann et al., J. Viral. 70:3198-3206, 1996; Reimann et al., J. Viral. 70:6922-6928, 1996), which allows a rapid determination of vaccine efficacy. The SHIV-89.6P challenge can be administered via both intrarectal and intravenous routes. SHIV-89.6 and SHIV-89.6P do not generate cross-neutralizing antibody.

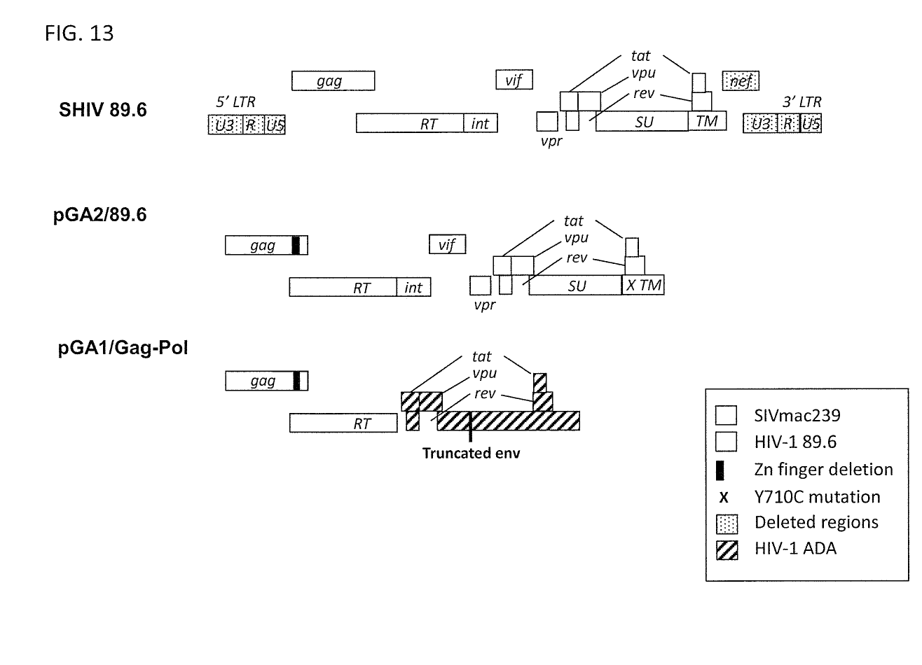

pGA2/89.6 (FIG. 13) has many of the design features of pGA2/JS2. Both express immunodeficiency virus VLPs: HIV-1 VLP in the case of pGA2/JS2, while the VLP expressed by pGA2/89.6 is a SHIV VLP. The gag-pol sequences in pGA2/89.6 are from SIV239, while the tat, rev, and env sequences are from HIV-1-89.6. pGA2/89.6 also differs from pGA2/JS2 in that the integrase, vif and vpr sequences have not been deleted, nor has the reverse transcriptase gene been inactivated by point mutations. Finally, the zinc fingers in NC have been inactivated by a deletion and not by point mutations.

pGA1/Gag-Po (FIG. 13) was also constructed to allow evaluation of the protective efficacy of a Gag-Pol expressing vector with the Gag-Pol-Env expresssing pGA2/89.6. This vector was constructed from pGA1/JS5 and pGA2/89.6.

### Example 12

### pGA2/89.6 SHIV Expression Versus pGA2/JS2 Expression

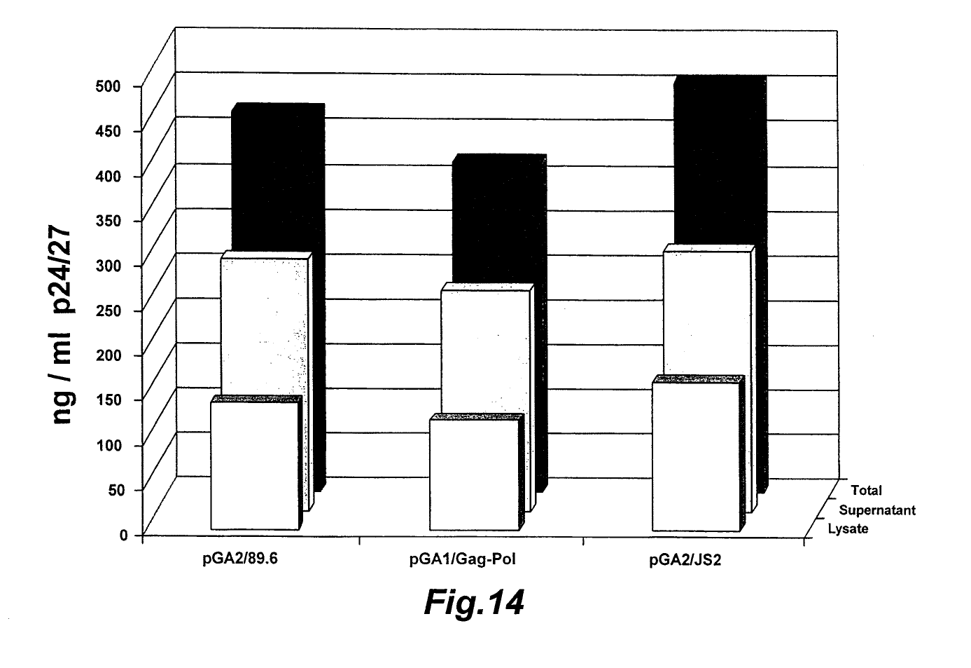

Both pGA2/89.6 and pGA1/Gag-Pol expressed levels of Gag that were similar to that expressed by pGA2/JS2. Comparative studies for expression were performed on transiently transfected 293T cells. Analyses of the lysates and supernatants of transiently transfected cells revealed that both plasmids expressed similar levels of capsid antigen (FIG. 14). The capsid proteins were quantified using commercial antigen capture ELISA kits for HIV-1 p24 and SIV p27.

### Example 13

### pGA2/89.6 SHIV Vaccine Protocol

A rhesus macaque model was used to investigate the ability of systemic DNA priming followed by a recombinant MVA (rMVA) booster to protect against a mucosalchallenge with the SHIV-89.6P challenge strain (Amara et al., Science 292:69-74, 2001). This model can be used to assess a variety of vaccine constructs, including those in which an rMVA construct is administered alone (i.e., without priming with a DNA vector), and those in which the antigens vary from those exemplified (or are obtained from other viral clades, such as clade AG; see the description of the IC-series of inserts described herein).

The DNA component of the vaccine (pGA2/89.6) was made as described in Example 11 and expressed eight immunodeficiency virus proteins (SIV Gag, Pol, Vif, Vpx, and Vpr and HIV Env, Tat, and Rev) from a single transcript using the subgenomic splicing mechanisms of immunodeficiency viruses. The rMVA booster (89.6-MVA) was provided by Dr. Bernard Moss (NIH) and expressed both the HIV 89.6 Env and the SIV239 Gag-Pol, inserted into deletion II and deletion III respectively of MVA, and under the control of vaccinia virus early/late promoters. The 89.6 Env protein lacked the C-terminal 115 amino acids of gp41. The modified HS promoter controlled the expression of both foreign genes.

The vaccination trial compared i.d. and i.m. administration of the DNA vaccine and the ability of a genetic adjuvant, a plasmid expressing macaque GM-CSF, to enhance the immune response raised by the vaccine inserts. Vaccination was by priming with DNA at 0 and 8 weeks and boosting with rMVA at 24 weeks. For co-delivery of a plasmid expressing GM-CSF, 1-100 μl i.d. inoculation was given with a solution containing 2.5 mg of pGA2/89.6 and 2.5 mg per ml of pGM-CSF.

Intradermal and intramuscular routes of delivery were compared for two doses, 2.5 mg and 250 μg of DNA. Four vaccine groups of six rhesus macaques were primed with either 2.5 mg (high-dose) or 250 μg (low-dose) of DNA by, as noted, intradermal or intramuscular routes using a needleless jet injection device (Bioject, Portland Oreg.). The 89.6-MVA booster immunization (2×10⁸ pfu) was injected with a needle both intradermally and intramuscularly. A control group included two mock immunized animals and two naive animals. The vaccination protocol is summarized in Table 5.

TABLE 5
Vaccination Trial

| Group, | | | Boost at | |
|---|---|---|---|---|
| (# macaque) | Prime at 0 and 8 weeks | Immunogen | 24 weeks | Immunogen |
| 1 (6) | i.d. bioject | 2.5 mg VLP DNA | i.d. + i.m. | MVA gag-pol-env |
| 2 (6) | i.m. bioject | 2.5 mg VLP DNA | i.d. + i.m. | MVA gag-pol-env |
| 3 (6) | i.d bioject | 250 μg VLP DNA | i.d. + i.m | MVA gag-pol-env |
| 4 (6) | i.m. bioject | 250 μg VLP DNA | i.d. + i.m | MVA gag-pol-env |
| 5 (6) | i.d. bioject | 2.5 mg gag-pol DNA | i.d. + i.m. | MVA gag-pol |
| 6 (6) | i.d. bioject | 250 μg gag-pol DNA | i.d. + i.m. | MVA gag-pol |
| 7 (6) | i.d bioject | 250 μg VLP DNA + | +i.d. + i.m. | MVA gag-pol-env |
| | | 250 μg GM-CSF DNA | | |
| 8 (5) | i.d. bioject | 2.5 mg control DNA | i.d. + i.m. | control MVA |
| | i.d. + i.m. control MVA | control MVA | | |
| 9 (4) | i.d., bioject | 250 μg control DNA + | +i.d. + i.m. | MVA gag-pol-env |
| | | 250 μg GM-CSF DNA | | |
| 10 (6) | i.d. + i.m. | MVA gag-pol-env | i.d. + i.m. | MVA gag-pol-env |

VLP DNA expresses all SHIV-89.6 proteins except Nef, truncated for LTRs, second zinc finger, mutated to express cell surface Env; gag-pol DNA expresses SIV mac 239 gag pol; MVA gag-pol-env expresses 89.6 truncated env and SIV mac 239 gag-pol; MVA gag-pol expresses SIVmac239 gag-pol; MVA dose is 1×10⁸ pfu.

Animals were challenged seven months after the rMVA booster to determine whether the vaccine generated long-term immunity. Because most HIV-1 infections are transmitted across mucosal surfaces, an intrarectal challenge was administered to test whether the vaccine could control a mucosal immunodeficiency virus challenge. The challenge stock (5.7×109 copies of viral RNA per ml) was produced in rhesus macaques by one intravenous followed by one intrarectal passage of the original SHIV-89.6P stock. Lymphoid cells were harvested from the intrarectally infected animal at peak viremia, CDS-depleted and mitogen-stimulated for stock production. Prior to intrarectal challenge, fasted animals were anesthetized (ketamine, 10 mg/kg) and placed on their stomach with the pelvic region slightly elevated. A feeding tube (8 Fr (2.7 mm)×16 inches (41 cm), Sherwood Medical, St. Louis, Mo.) was inserted into the rectum for a distance of 15-20 cm. A syringe containing 20 intrarectal infectious doses in 2 ml of RPMI-1640 plus 10% fetal bovine serum (PBS) was attached to the tube and the inoculum slowly injected into the rectum. Following delivery of the inoculum, the feeding tube was flushed with 3.0 ml of RPMI without fetal calf serum and then slowly withdrawn. Animals were left in place, with pelvic regions slightly elevated, for a period of ten minutes following the challenge.

### Example 14

### Vaccine-raised T-cell responses

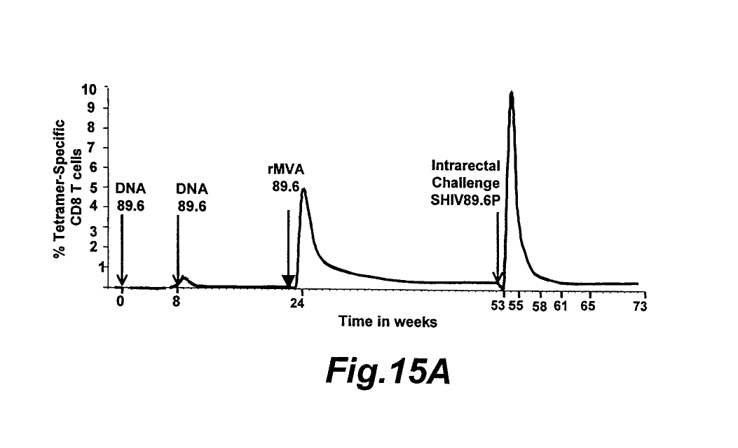

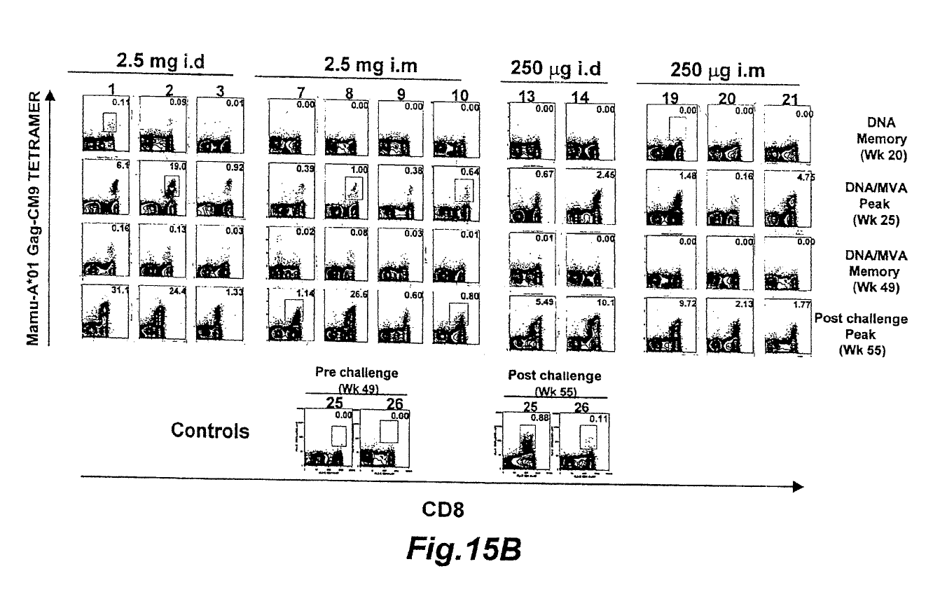

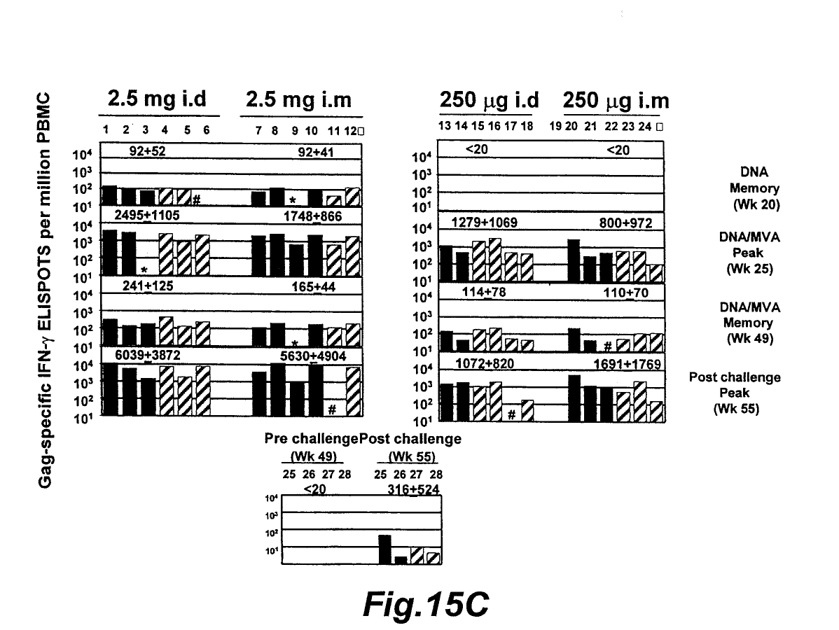

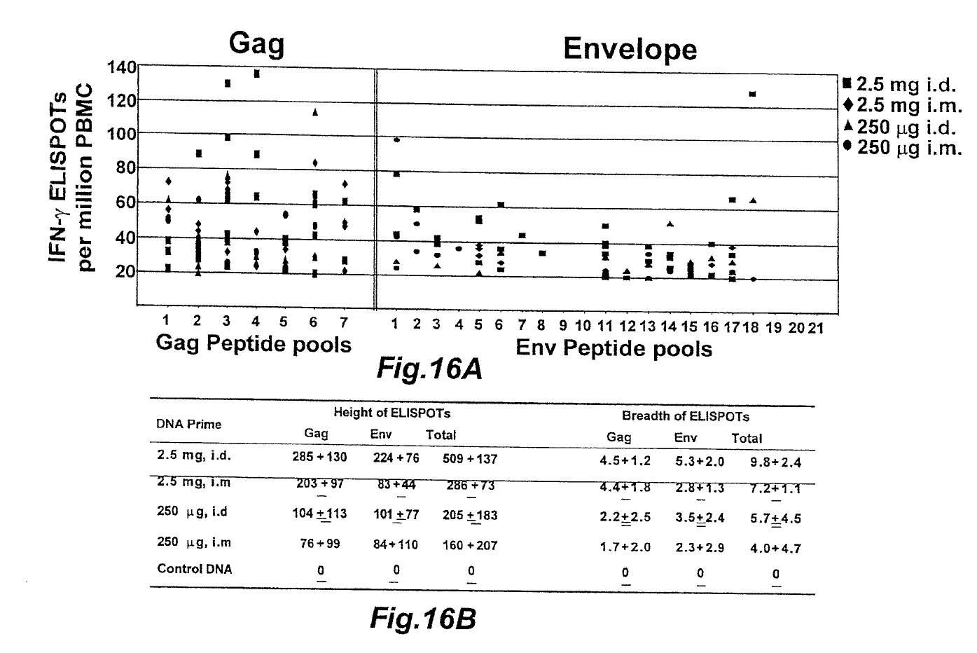

DNA priming followed by rMVA boosting generated high frequencies of virus specific T cells that peaked at one week following the rMVA booster (FIG. 15A). The frequencies of T cells recognizing the Gag-CM9 epitope were assessed using MamuA*01-tetramers (FIG. 15B), and the frequencies of T cells recognizing epitopes throughout Gag and Env, using pools of overlapping Gag and Env peptides and using anenzyme linked immunospot (ELISPOT) assay (FIG. 15C).

For tetramer analyses, approximately 1×10⁶ peripheral blood mononucleocytes (PBMC) were surface stained with antibodies to CD3 (FN-18, Biosource International, Camarillo, Calif.), CD8 (SKI, Becton Dickinson, San Jose, Calif.), and the Gag-CM9 (CTPYDINQM)-Mamu-A*01 tetramer conjugated to FITC, PerCP and APC respectively, in a volume of 100 μl at 8-10° C. for 30 minutes. Cells were washed twice with cold PBS containing 2% FBS, fixed with 1% paraformaldehyde in PBS and analyses acquired within 24 hours on a FACS caliber (Becton Dickinson, San Jose, Calif.). Cells were initially gated on lymphocyte populations using forward scatter and side scatter and then on CD3 cells. The CD3 cells were then analyzed for CDS and tetramer-binding cells. Approximately 150,000 lymphocytes were acquired for each sample. Data were analyzed using FloJo software (Tree Star, Inc. San Carlos, Calif.).

For IFN-γ ELISPOTs, MULTISCREEN™ 96-well filtration plates (Millipore Inc. Bedford, Mass.) were coated overnight with anti-human IFN-γ antibody (Clone B27, Pharmingen, San Diego, Calif.) at a concentration of 2 μg/ml in sodium bicarbonate buffer (pH 9.6) at 8-10° C. Plates were washed two times with RPMI medium then blocked for one hour with complete medium (RPMI containing 10% FBS) at 37° C. Plates were washed five more times with plain RPMI medium and cells were seeded in duplicate in 100 μl complete medium at numbers ranging from 2×10⁴ to 5×10⁵ cells per well. Peptide pools were added to each well to a final concentration of 2 μg/ml of each peptide in a volume of 100 μl in complete medium. Cells were cultured at 37° C. for about 36 hours under 5% CO₂. Plates were washed six times with wash buffer (PBS with 0.05% Tween-20) and then incubated with 1 μg of biotinylated anti-human IFN-γ antibody per ml (clone 7-86-1, Diapharma Group Inc., West Chester, Ohio) diluted in wash buffer containing 2% FBS. Plates were incubated for 2 hrs at 37° C. and washed six times with wash buffer. Avidin-HRP (Vector Laboratories Inc, Burlingame, Calif.) was added to each well and incubated for 30-60 min at 37° C. Plates were washed six times with wash buffer and spots were developed using stable DAB as substrate (Research Genetics Inc., Huntsville, Ala.). Spots were counted using a stereo dissecting microscope. An ovalbumin peptide (SIINFEKL (SEQ ID NO: ______)) was included as a control in each analysis. Background spots for the ovalbumin peptide were generally <5 for 5×10⁵ PBMCs. This background when normalized for 1×10⁶ PBMC is <10. Only ELISPOT counts of twice the background (2:20) were considered significant. The frequencies of ELISPOTs are approximate because different dilutions of cells have different efficiencies of spot formation in the absence of feeder cells. The same dilution of cells was used for all animals at a given time point, but different dilutions were used to detect memory and peak effector responses.

Simple linear regression was used to estimate correlations between post-booster and post-challenge ELISPOT responses, between memory and post-challenge ELISPOT responses, and between log viral loads and ELISPOT frequencies in vaccinated groups. Comparisons between vaccine and control groups were performed by means of 2-samplet-tests using log viral load and log ELISPOT responses. Comparisons of ELISPOTs or log viral loads between A*01 and non-A*01 macaques were done using 2-sample t-tests. Two-way analyses of variance were used to examine the effects of dose and route of administration on peak DNA/MVA ELISPOTs, memory DNA/MVA ELISPOTs, and on logarithmically transformed Gag antibody data.

Gag-CM9 tetramer analyses were restricted to macaques that expressed the Mamu-A *01 histocompatibility type, whereas ELISPOT responses did not depend on a specific histocompatibility type. Temporal T cell assays were designed to score both the acute (peak of effector cells) and long-term (memory) phases of the T cell response, as shown in FIG. 15A. As expected, the DNA immunizations raised low levels of memory cells that expanded to high frequencies within one week of the rMV A booster.

In Mamu-A*01 macaques, cells specific to the Gag-CM9 epitope expanded to frequencies as high as 19% of total CD8 T cells (see FIG. 15B, animal 2). This peak of specific cells underwent a >10-fold contraction into the DNA/MVA memory pool, as shown in FIGS. 15A and 15B.

ELISPOTs for three pools of Gag peptides also underwent a major expansion (frequencies up to 4000 spots for 1×10⁶ PBMC) before contracting into the DNA/MVA memory response, as shown in FIG. 15C. The frequencies of ELISPOTs were the same in macaques with and without the A*01 histocompatibility type (P>0.2.). At both peak and memory phases of the vaccine response, the rank order for the height of the ELISPOTs in the different vaccine groups was 2.5 mg i.d>2.5 mg i.m.>250 μg i.d.>250 μg i.m.(FIG. 15C). The IFN-γ-ELISPOTs included both CD4 and CD8 cells. Gag-CM9-specific CD8 cells had good lytic activity following restimulation with peptide.

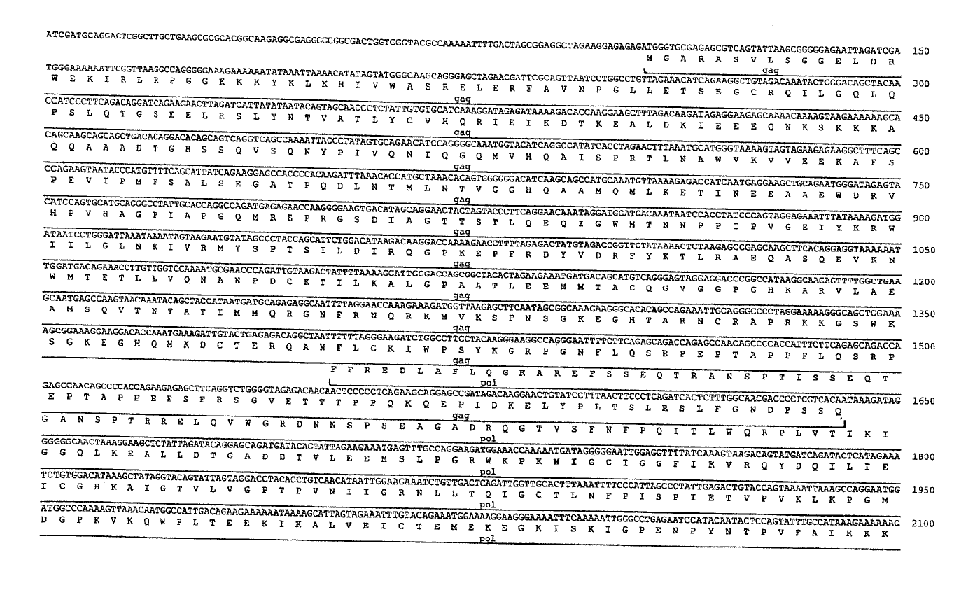

In the outbred population of animals, pools of peptides throughout Gag and Env stimulated IFN-γ-ELISPOTs (FIG. 16A). The breadth of the cellular response was tested 25 weeks after the rMVA boost, a time when vaccine-raised T cells were in memory. Seven out of seven pools of Gag peptides and 16 out of 21 pools of Env peptides (approximately seven 22-mers overlapping by 12) representing about 70 amino acids of Gag sequence, and 21 pools of Env peptides (approximately ten 15-mers overlapping by 11) representing about 40 amino acids of Env sequence were recognized by T cells in vaccinated animals. Assays for the first 12 weeks post challenge had a background of 1000 copies of RNA per ml of plasma. Animals with loads below 1000 were scored with a load of 500. For weeks 16 and 20, the background for detection was 300 copies of RNA/ml. Animals with levels of virus below 300 were scored at 300.

Of the five Env pools that were not recognized, two have been recognized in a macaque DNA/MVA vaccine trial at the U.S. Centers for Disease Control. The remaining three pools (19-21) had been truncated in our immunogens and served as negative controls.

Gag and Env ELISPOTs had, overall, similar frequencies in the DNA/MVA memory response (FIG. 16B). The greatest breadth of response was in high-dose i.d. DNA-primed animals where, on average, 10 peptide pools (4.5 Gag and 5.3 Env) were recognized. The rank order of the vaccine groups for breadth was the same as for the peak DNA/MY A response: 2.5 mg i.d.>2.5 mg i.m.>250 μg i.d.>250 μg i.m.

### Example 15

### Challenge and protection against AIDS

The highly pathogenic SHIV-89.6P challenge was administered intrarectally seven months after the rMVA booster, when vaccine-raised T cells were in memory, as shown in Fig ISA.

Determination of SHIV Copy Number:

Viral RNA from 150 μl of ACD anticoagulated plasma was directly extracted with the QIAAMP™ viral RNA kit (Qiagen), eluted in 60 μl A VE buffer, and frozen at −80° C. until SHIV RNA quantitation was performed. 5 μl of purified plasma RNA was reverse transcribed in a final 20 μl volume containing 50 mM KCl, 10 mM Tris-HCl, pH 8.3, 4 mM MgCl₂, 1 mM each dNTP, 2.5 μM random hexamers, 20 units MultiScribe RT, and 8 units RNase inhibitor. Reactions were incubated at 25° C. for 10 min., followed by incubation at 42° C. for 20 min. and inactivation of reverse transcriptase at 99° C. for 5 min. The reaction mix was adjusted to a final volume of 50 μL containing 50 mM KCl, 10 mM Tris-HCl, pH 8.3, 4 mM MgCl₂, 0.4 mM each dNTP, 0.2 μM forward primer, 0.2 μM reverse primer, 0.1 μM probe and 5 units AMPLITAQ™ Gold DNA polymerase (Perkin Elmer AppliedBiosystems, Foster City, Calif.). The primer sequences within a conserved portion of the SIV gag gene are the same as those described by Staprans et al. (In Viral Genome Methods, K. Adolph, Ed., CRC Press, Boca Raton, Fla., pp. 167-184, 1996).

A Perkin Elmer Applied Biosystems 7700 Sequence Detection System was used with the PCR profile: 95° C. for 10 minutes, followed by 40 cycles at 93° C. for 30 seconds, and a hold at 59.5° C. for 1 minute. PCR product accumulation was monitored using the 7700 sequence detector and a probe to an internal conserved gag genesequence, where FAM and Tamra denote the reporter and quencher dyes. SHIV RNAcopy number was determined by comparison to an external standard curve consisting of virion-derived SIVmac239 RNA quantified by the SIV bDNA method (Bayer Diagnostics, Emeryville, Calif.). All specimens were extracted and amplified in duplicate, with the mean result reported. With a 0.15-ml plasma input, the assay has a sensitivity of 10³ copies RNA/ml plasma, and a linear dynamic range of 10³ to 10⁸ RNA copies (R2=0.995). The intra-assay coefficient of variation is <20% for samples containing >10⁴SHIV RNA copies/ml, and <25% for samples containing 10³-10⁴ SHIV RNA copies/ml. In order to more accurately quantitate low SHIV RNA copy number in vaccinated animals at weeks 16 and 20, the following modifications to increase the sensitivity of the SHIV RNA assay were made: 1) Virions from <1 ml of plasma were concentrated by centrifugation at 23,000 g, 10° C. for 150 minutes and viral RNA was extracted; 2) A one step RT-PCR method was used. Absolute SHIV RNA copy numbers were determined by comparison to the same SIVmac239 standards. These changes provided a reliable quantitation limit of 300 SHIV RNA copies/ml, and gave SHIV RNA values that were highly correlated to those obtained by the first method used (r=0.91, p<0.0001).

Challenge Results:

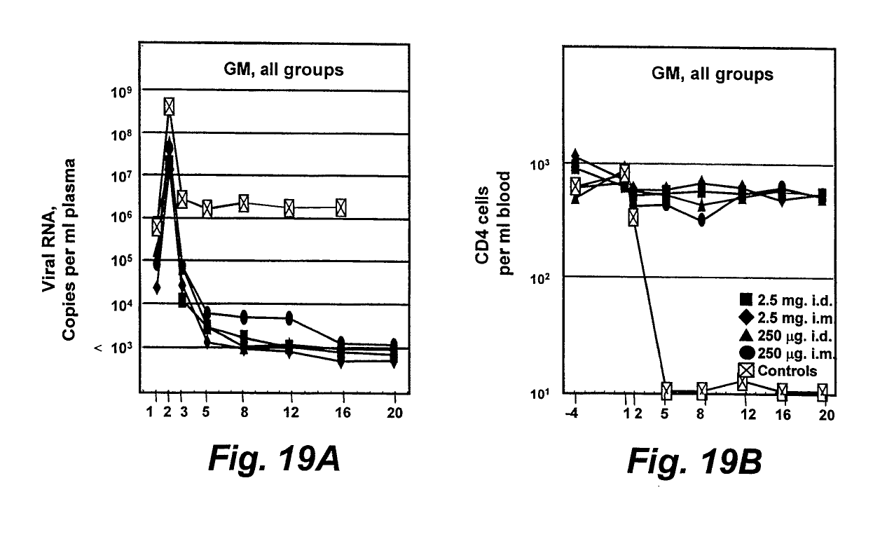

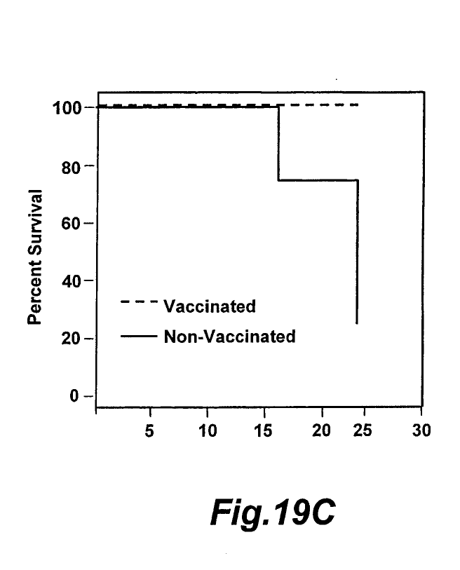

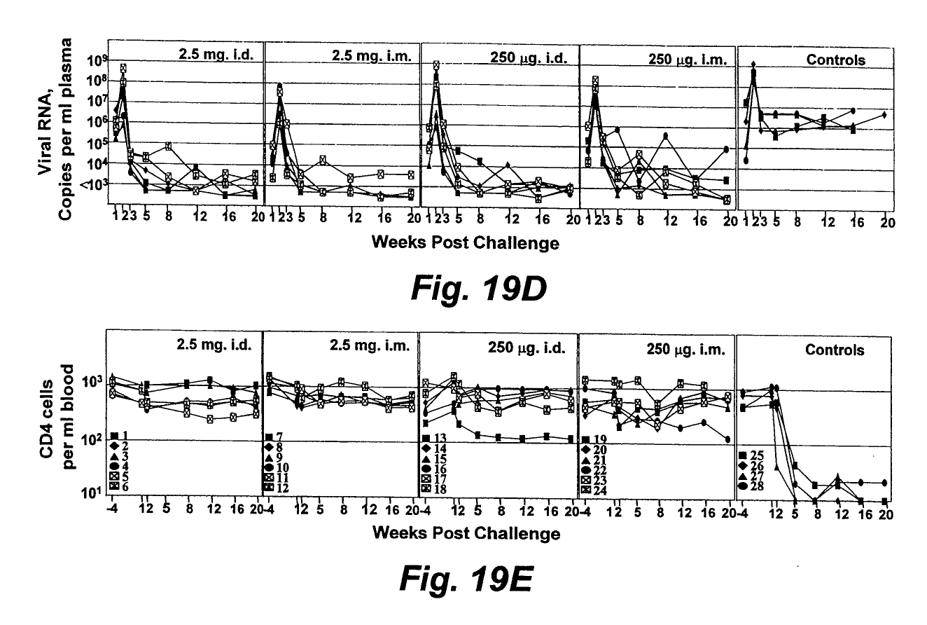

The challenge infected all of the vaccinated and control animals. However, by two weeks post-challenge, titers of plasma viral RNA were at least 10-fold lower in the vaccine groups (geometric means of 1×10⁷ to 5×10⁷) than in the control animals (geometric mean of 4×10⁸), as shown in FIG. 19A. By 8 weeks postchallenge, both high-dose DNA-primed groups and the low-dose i.d. DNA-primed group had reduced their geometric mean loads to about 1000 copies of viral RNA per ml. At this time the low-dose i.m. DNA-primed group had a geometric mean of 6×10³ copies of viral RNA and the non-vaccinated controls, a geometric mean of 2×10⁶. By 20 weeks post-challenge, even the low-dose i.m. group had reduced its geometric mean copies of viral RNA to 1000. At this time, the unvaccinated controls were succumbing to AIDS. Among the 24 vaccinated animals, only one animal, in the low dose i.m. group, had intermittent viral loads above 1×10⁴ copies per ml, as shown in FIG. 19D.

The rapid reduction of viral loads protected the vaccinated macaques against the loss of CD4 cells and the rapid onset of AIDS, as shown in FIGS. 19B, 19C, and 19E. By5 weeks post-challenge, all of the non-vaccinated controls had undergone the profound depletion of CD4 cells that is characteristic of SHIV-89.6P infections (FIG. 19B). All of the vaccinated animals maintained their CD4 cells with the exception of animal 22 (see above), which underwent a slow CD4 decline (FIG. 19E). By 23 weeks post-challenge, three of the four control animals had succumbed to AIDS (FIG. 19C). These animals had variable degrees of enterocolitis with diarrhea, cryptosporidiosis, colicystitis, enteric campylobacter infection, splenomegaly, lymphadenopathy, and SIV-associated giant cell pneumonia. In contrast, all 24 vaccinated animals maintained their health.

Intracellular Cytokine Assays:

Approximately 1×10⁶ PBMC were stimulated for one hour at 37° C. in 5 ml polypropylene tubes with 100 μg of Gag-CM9 peptide (CTPYDINQM) per ml in a volume of 100 μl RPMI containing 0.1% BSA and antihuman CD28 and anti-human CD49d (Pharmingen, Inc. San Diego, Calif.) costimulatory antibodies (1 μg/ml). 900 μl RPMI containing 10% FBS and monensin (10 μg/ml) was added and the cells cultured for an additional 5 hrs at 37° C. at an angle of 5 degrees under 5% CO₂. Cells were surface stained with antibodies to CDS conjugated to PerCP (cloneSKI, Becton Dickinson) at 8°-10° C. for 30 min., washed twice with cold PBS containing2% FBS, fixed and permeabilized with Cytofix/Cytoperm solution (Pharmingen, Inc.). Cells were then incubated with antibodies to human CD3 (clone FN-18, Biosource International, Camarillo, Calif.) and IFN-γ (Clone B27, Pharmingen) conjugated to FITC and PE, respectively, in Perm wash solution (Pharmingen) for 30 min at 4° C. Cells were 49 washed twice with Perm wash, once with plain PBS, and resuspended in 1% paraformaldehyde ein PBS. Approximately 150,000 lymphocytes were acquired on the FACScaliber and analyzed using FLOJO™ software.

Proliferation assay: Approximately 2×10⁵ PBMC were stimulated with appropriate antigen in triplicate in a volume of 200 μl for five days in RPMI containing 10% PCS at 37° C. under 5% CO₂. Supernatants from 293T cells transfected with the DNA expressing either SHIV-89.6 Gag and Pol or SHIV-89.6 Gag, Pol and Env were used directly as antigens. Supernatants from mock DNA (vector alone) transfected cells served as negative controls. On day 6, cells were pulsed with 1 μCi of tritiated-thymidine per well for 16-20 hrs. Cells were harvested using an automated cell harvester (TOMTEC, Harvester 96, Model 1010, Hamden, Conn.) and counted using a Wallace 1450MICROBETA Scintillation counter (Gaithersburg, Md.). Stimulation indices are the counts of tritiated-thymidine incorporated in PBMC stimulated with 89.6 antigens divided by the counts of tritiated-thymidine incorporated by the same PBMC stimulated with mock antigen.

Post-Challenge T Cell Results:

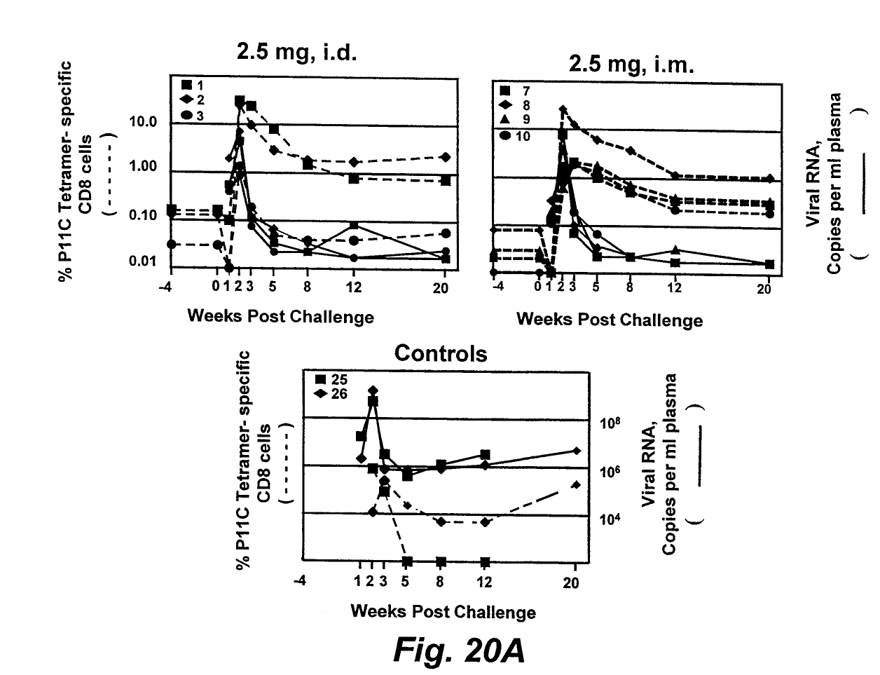

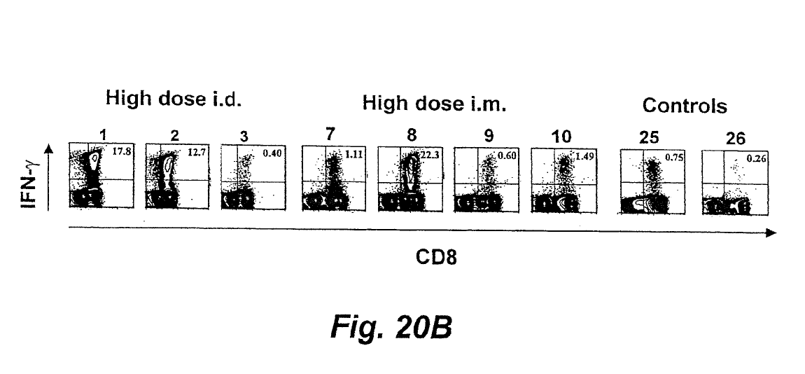

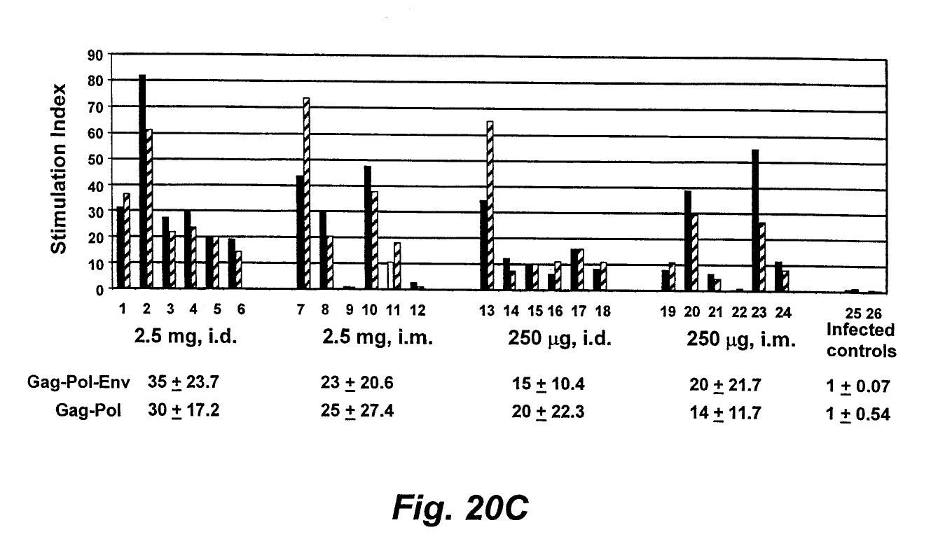

Containment of the viral challenge was associated with a burst of antiviral T cells, as shown in FIGS. 15 and 20A. At one-week postchallenge, the frequency of tetramer+ cells in the peripheral blood had decreased, potentially reflecting the recruitment of specific T cells to the site of infection. However, by two weeks post-challenge, tetramer+ cells in the peripheral blood had expanded rapidly, to frequencies as high, or higher, than after the MVA booster (FIGS. 15, 20A). The majority of the tetramer+ cells produced IFN-γ in response to a 6-hour stimulation with peptide Gag-CM9 (FIG. 20B) and did not have the “stunned” IFN-γ negative phenotype sometimes observed in chronic viral infections. The post-challenge burst of T cells contracted concomitant with the decline of the viral load. By 12 weeks postchallenge, virus-specific T cells were present at approximately one tenth of their peak height (FIGS. 15A and 20A). The height of the peak DNA/MVA-induced ELISPOTs presaged the height of the post-challenge T cell response as measured by ELISPOTs (r=+0.79, P<0.0001). In contrast to the vigorous secondary response in the vaccinated animals, the naive animals mounted a modest primary response (FIGS. 15B, 15C and 20A). Tetramer+ cells peaked at less than 1% of total CDS cells (FIG. 20A), and IFN-γ-50 producing T cells were present at a mean frequency of about 300 as opposed to the much higher frequencies of 1000 to 6000 in the vaccine groups (FIG. 15C) (P<0.05). The tetramer+ cells in the control group, like those in the vaccine group, were largely IFN-γ producing following stimulation with the Gag-CM9 peptide, shown in FIG. 20B. By 12 weeks post challenge, 3 of the 4 controls had undetectable levels of IFN-γ -producing T cells. This rapid loss of anti-viral CDS cells in the presence of high viral loads may reflect the lack of CD4 help.

T cell proliferative responses demonstrated that virus-specific CD4 cells had survived the challenge and were available to support the antiviral immune response, as illustrated in FIG. 20C. At 12 weeks post-challenge, mean stimulation indices for Gag Pol-Env or Gag-Pol proteins ranged from 35 to 14 in the vaccine groups but were undetectable in the control group. Consistent with the proliferation assays, intracellular cytokine assays demonstrated the presence of virus-specific CD4 cells in vaccinated but not control animals. The overall rank order of the vaccine groups for the magnitude of the proliferative response was 2.5 mg i.d.>2.5 mg i.m.>250 μg i.d.>250 μg i.m.

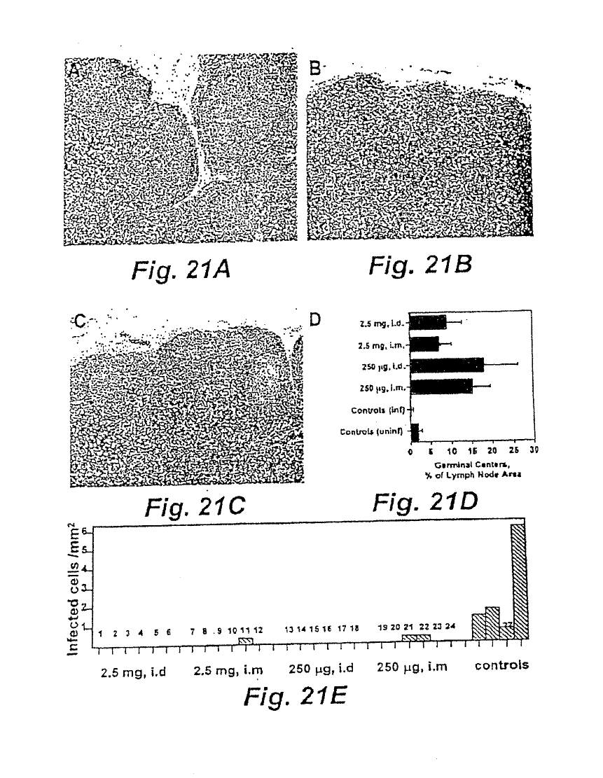

Preservation of lymph nodes: At 12 weeks post-challenge, lymph nodes from the vaccinated animals were morphologically intact and responding to the infection whereas those from the infected controls had been functionally destroyed, as shown in FIGS. 21A-C. Nodes from vaccinated animals contained large numbers of reactive secondary follicles with expanded germinal centers and discrete dark and light zones (FIG. 21A). By contrast, lymph nodes from the non-vaccinated control animals showed follicular and paracortical depletion (FIG. 21B), while those from unvaccinated and unchallenged animals displayed normal numbers of minimally reactive germinal centers (FIG. 21C). Germinal centers occupied<0.05% of total lymph node area in the infected controls, 2% of the lymph node area in the uninfected controls, and up to 18% of the lymph node area in the vaccinated groups, shown in FIG. 21D. The lymph node area occupied by germinal centers was about two times greater for animals receiving low-dose DNA priming than for those receiving high-dose DNA priming, suggesting more vigorous immune reactivity in the low-dose animals (FIG. 21D).

At 12 weeks post-challenge, in situ hybridization for viral RNA revealed rare virus-expressing cells in lymph nodes from 3 of the 24 vaccinated macaques, whereas virus-expressing cells were readily detected in lymph nodes from each of the infected control animals (shown in FIG. 21E). In the controls, which had undergone a profound depletion in CD4 T cells, the cytomorphology of infected lymph node cells was consistent with a macrophage phenotype.

Temporal antibody response: ELISAs for total anti-Gag antibody used bacterial produced SIV gag p27 to coat wells (2 μg per ml in bicarbonate buffer). ELISAs for antiEnv antibody used 89.6 Env produced in transiently transfected 293T cells and captured with sheep antibody against Env (catalog number 6205; International Enzymes, Fairbrook CA). Standard curves for Gag and Env ELISAs were produced using serum from a SHIV-89.6-infected macaque with known amounts of anti-Gag or anti-Env IgG. Bound antibody was detected using goat anti-macaque IgG-PO (catalog# YNGMOIGGFCP, Accurate Chemical, Westbury, N.Y.) and TMB substrate (Catalog# T3405, Sigma Chemical Co., St. Louis, Mo.). Sera were assayed at 3-fold dilutions in duplicate wells. Dilutions of test sera were performed in whey buffer (4% whey and 0.1% tween20 in 1×PBS). Blocking buffer consisted of whey buffer plus 0.5% non-fat dry milk. Reactions were stopped with 2M H₂SO₄ and the optical density read at 450 nm. Standard curves were fitted and sample concentrations were interpolated as μg of antibody per ml of serum using SOFTmax 2.3 software (Molecular Devices, Sunnyvale, Calif.).

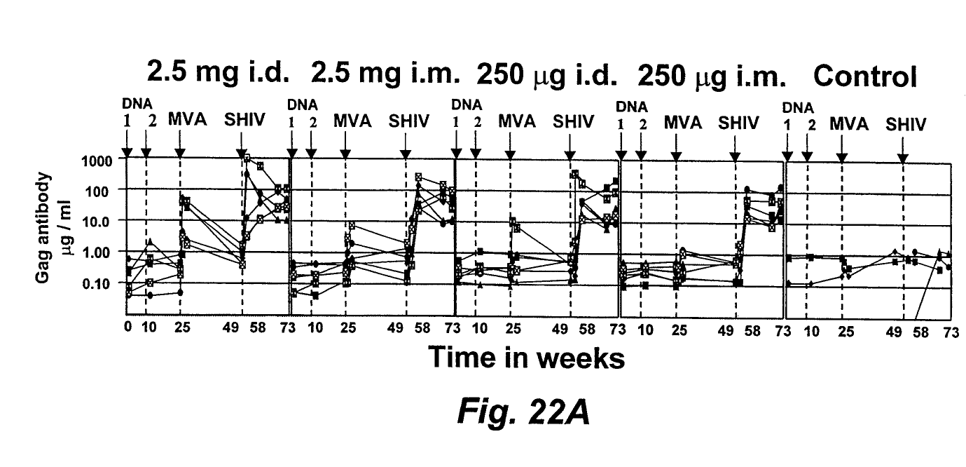

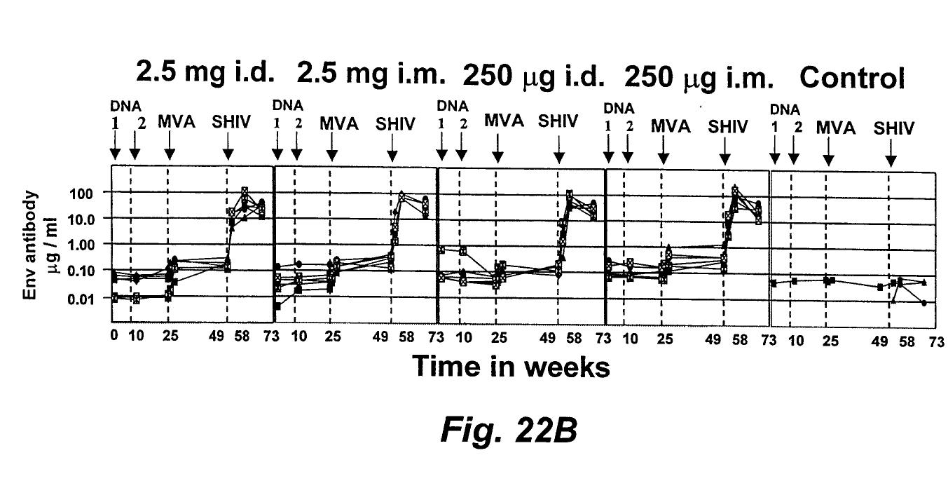

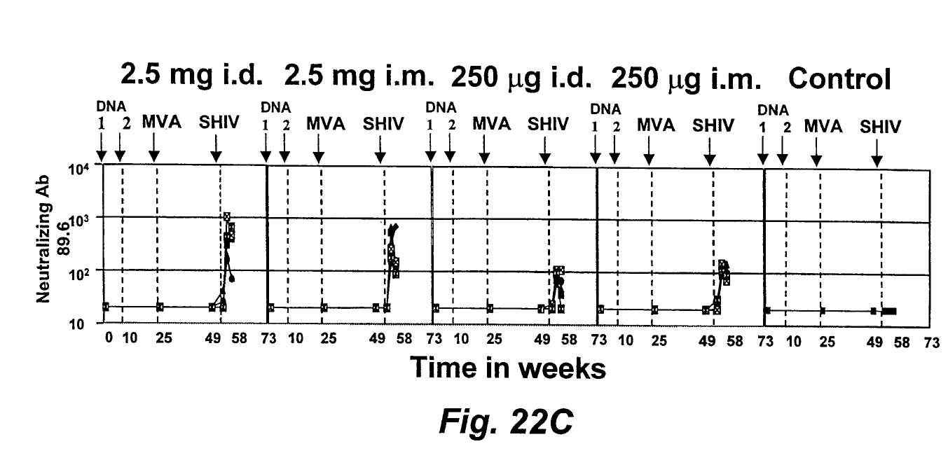

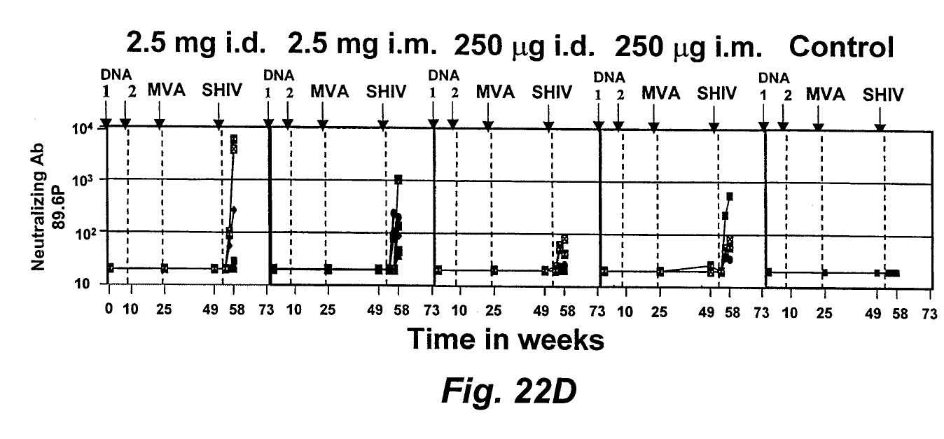

Results showed that the prime/boost strategy raised low levels of anti-Gag antibody and undetectable levels of anti-Env antibody, as shown in FIGS. 22A-22D. However, post-challenge, antibodies to both Env and Gag underwent anamnestic responses with total Gag antibody approaching 1 mg per ml and total Env antibody approaching 100 μg per ml, as shown in FIGS. 22A and 22B.

By two weeks post-challenge, neutralizing antibodies for the 89.6 immunogen, but not the SHIV-89.6P challenge, were present in the high-dose DNA-primed groups (geometric mean titers of 352 in the i.d. and 303 in the i.m. groups) (FIG. 22C). By 5 weeks post-challenge, neutralizing antibody to 89.6P had been generated (geometric mean titers of 200 in the high-dose i.d. and 126 in the high-dose i.m. group) (FIG. 22D) and neutralizing antibody to 89.6 had started to decline. Thus, priming of an antibody response to 89.6 did not prevent a B cell response leading to neutralizing antibody for 52SHIV-89.6P. By 16 to 20 weeks post-challenge, antibodies to Gag and Env had fallen in most animals, as shown in FIGS. 22A and 22B, consistent with the control of the virus infection.

T Cells Correlate with Protection:

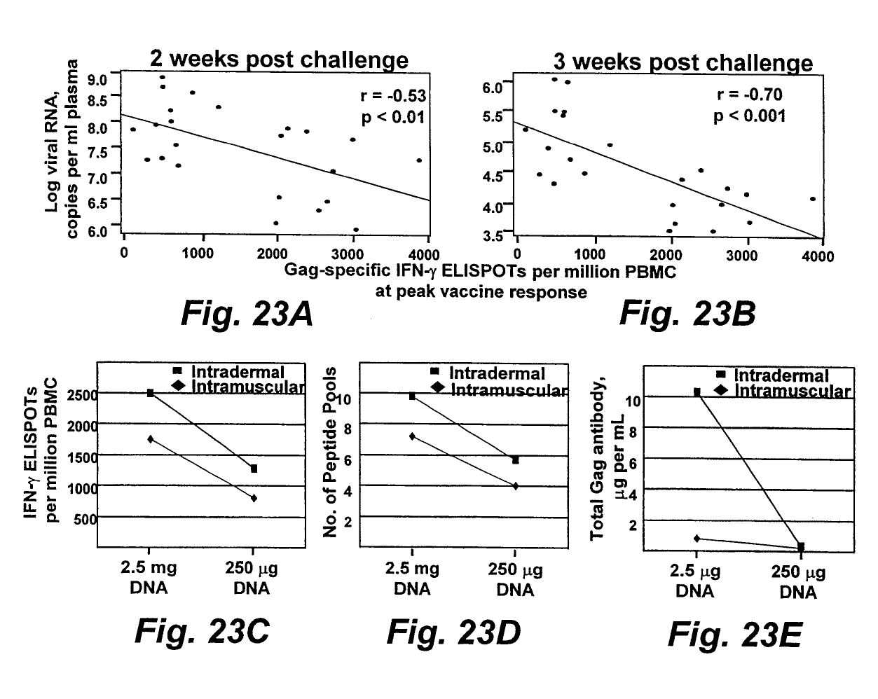

The levels of plasma viral RNA at both two and three weeks post-challenge correlated inversely with the peak pre-challenge frequencies of DNA/MVA-raised IFN-γ ELISPOTs (r=−0.53, P=0.008 and r=−0.70, P=0.0002 respectively) [(FIG. 23A)], as shown in FIGS. 23A and 23B. These correlations were observed during the time the immune response was actively reducing the levels of viremia. At later times post-challenge, the clustering of viral loads at or below the level of detection precluded correlations. Correlations also were sought between viral load and post-challenge ELISPOT, proliferative, and neutralizing antibody responses. The levels of IFN-γ ELISPOTS at two weeks post-challenge correlated with the viral load at 3 weeks post-challenge (r=−0.51, P=0.009). Post-challenge proliferative and neutralizing antibody responses did not correlate with viral loads.

Dose and Route:

The dose of DNA had significant effects on both cellular and humoral responses (P<0.05) while the route of DNA administration had a significant effect only on humoral responses, as illustrated in FIGS. 23C-23E. The intradermal route of DNA delivery was about 10 times more effective than the intramuscular route for generating antibody to Gag (P=0.02) (FIG. 23E). Intradermal DNA injections were about 3 times more effective than intramuscular DNA injections at priming the height and breadth of virus-specific T cells, as shown in FIGS. 23C and 23D. However, these differences were not significant (height, P=0.2; breadth, P=0.08).

The route and dose of DNA had no significant effect on the level of protection. At 20 weeks post-challenge, the high-dose DNA-primed animals had slightly lower geometric mean levels of viral RNA (7×10² and 5×10²) than the low-dose DNA-primedanimals (9×10² and 1×10³). The animal with the highest intermittent viral loads (macaque 22) was in the low dose i.m.-primed group, shown in FIG. 19D. Thus, the low dose i.m.-primed group, which was slow to control viremia, as shown in FIG. 19A, may have poorer long term protection. The breadth of the response did not have an immediate effect on the containment of viral loads, but may ultimately affect the frequency of viral escape.

These results show that a multiprotein DNA/MY A vaccine can raise a memory immune response capable of controlling a highly virulent mucosal immunodeficiency virus challenge. The levels of viral control are more favorable than have been achieved using only DNA or rMVA vaccines (Egan et al., (2000); Ourmanov et al., (2000)) and comparable to those obtained for DNA immunizations adjuvanted with interleukin-2(Barouch et al., Science 290:486-492, 2000). The previous studies have used more than three vaccine inoculations. None have used mucosal challenges, and most have challenged at peak effector responses and not allowed a prolonged post vaccination period to test for “long term” efficacy as were done in our study. The results described in the above Examples 1-15 demonstrate that vaccine-raised T cells, as measured by IFN-γ ELISPOTs, are a correlate for the control of viremia. This relatively simple assay is useful for the preclinical evaluation of DNA and MVA immunogens for HIV-1, and can be used as a marker for the efficacy of clinical trials in humans. The DNA/MVA vaccine did not prevent infection. Rather, the vaccine controlled the infection, rapidly reducing viral loads to near or below 1000 copies of viral RNA per ml of blood. Containment, rather than prevention of infection, affords the virus the opportunity to establish a chronic infection (Chun et al., Proc. Natl. Acad. Sci USA 95:8869-8873, 1998). Nevertheless, by rapidly reducing viral loads, a multiprotein DNA/MVA vaccine will extend the prospect for long-term non-progression and limit HIV transmission.

### Example 16

### Gag-Pol Vaccine Trial

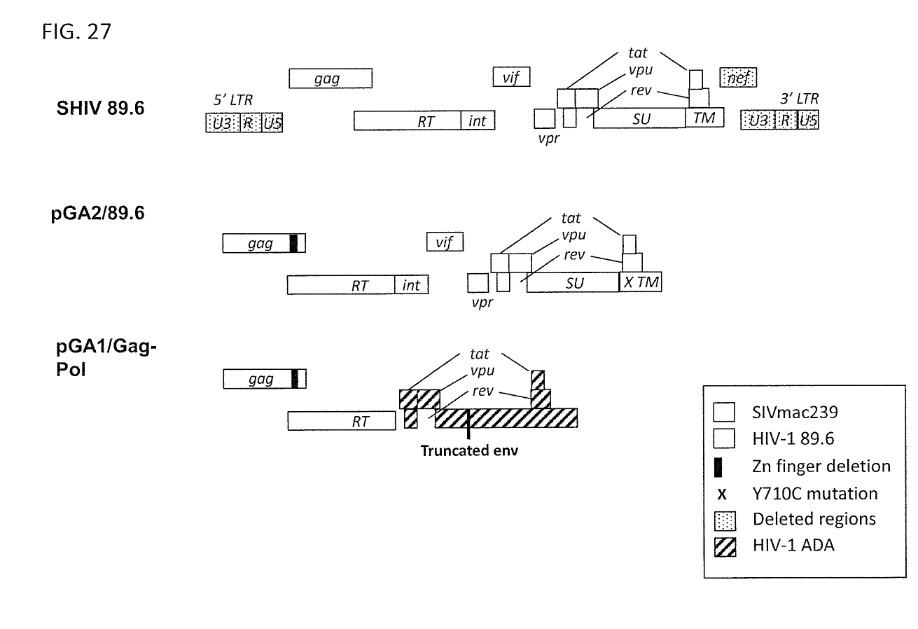

A trial using Gag-Pol rather than Gag-Pol-Env expressing immunogens was conducted to determine the importance of including Env in the vaccine. Constructs used in this study are shown in FIG. 27. A vaccine not having Env offers certain advantages in the field, such as allowing the screening for anti-Env antibody as a marker for infection. This trial used pGA1/Gag-Pol and a rMVA expressing the Gag-Pol sequences of SIY239 (MVA/Gag-Pol) supplied by Dr. Bernard Moss (NIH-NIAID).

The “Gag-Pol” immunogens pGA2/89.6 and MVA/89.6 were administered using the schedule described in Example 13 above (see Table 4, Groups 5 and 6). Doses of DNA, 2.5 mg and 250 μg, were used to prime a high dose and a low dose group respectively and administration was via an intradermal route. As in the vaccine trial described in Examples 13-15, two or three Mamu A*01 macaques were included in each trial group. T cell responses were followed for those specific for the pl lc-m epitopeusing the pl lc-m tetramers and using ELISPOTs stimulated by pools of overlapping peptides, as described in the above Examples 13-15.

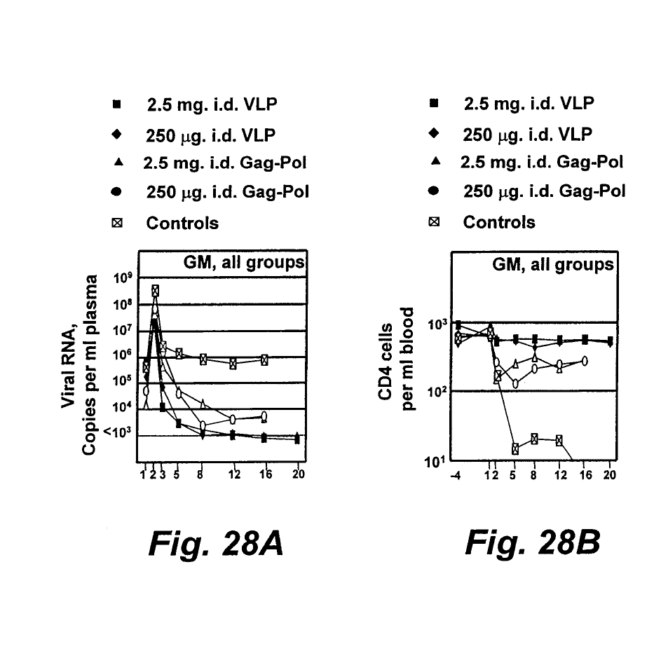

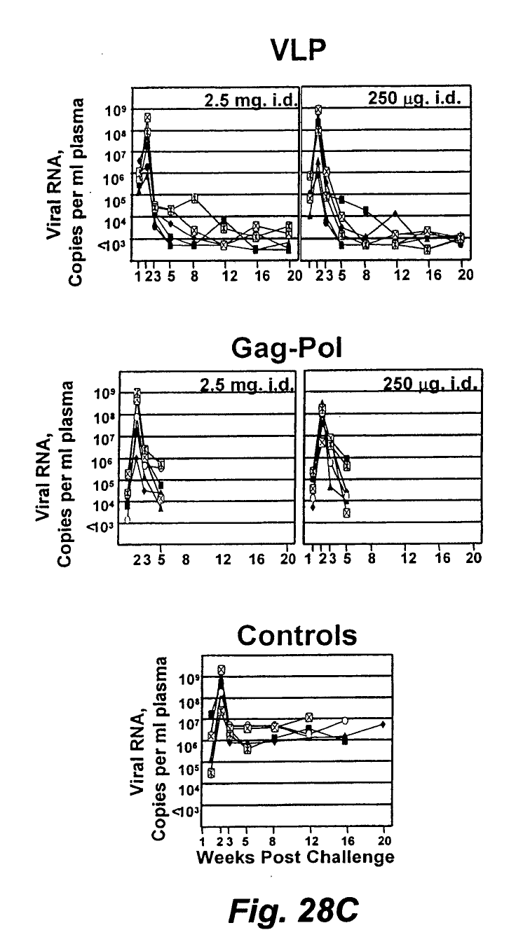

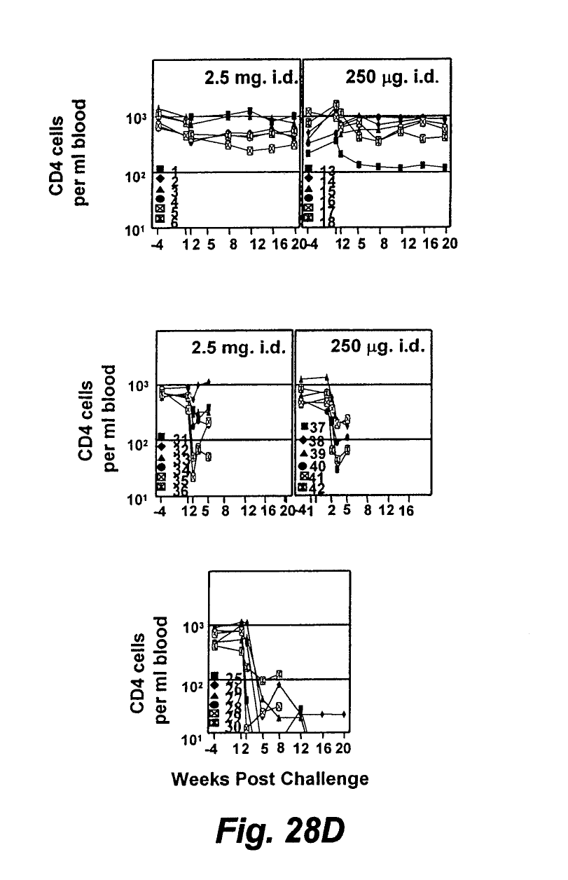

Following immunization, vaccine recipients showed anti-Gag T cell responses similar to those observed in the Gag-Pol-Env vaccine trial, as shown in FIGS. 28A-28E. Animals were challenged intrarectally with SHIV-89.6P at 7.5 months following the rMVA booster. In contrast to the Gag-Pol-Env vaccine protocol, which protected animals against the rapid loss of CD4 cells, the Gag-Pol animals uniformly lost CD4 cells (FIGS. 28B and 28E). This loss was most pronounced in the group receiving the low dose i.d.DNA prime. Consistent with the loss of CD4 cells, the Gag-Pol DNA-immunized groups were also less effective at reducing their viral loads than the Gag-Pol-Env groups (FIGS. 28A and 28D). Geometric mean viral loads for these groups were 10-100-fold higher at 3 weeks post challenge and 10 fold higher at 5 weeks post challenge. These results demonstrate that the Env gene plays an important role in protecting CD4 cells and reducing the levels of viral RNA in challenged animals. The results also show that GagPol-Env DNA/MVA vaccines function more effectively than Gag-Pol DNA/MVA vaccines in protecting recipients against a virulent challenge.

### Example 17

### Measles Inserts

Previous studies showed that antibody could be raised to intracellular but not the plasma membrane protein. Review of the literature suggests that some plasma membrane proteins are like intracellular proteins in being able to support the raising of antibody in the presence of maternal antibody. Thus it will be possible to engineer the measles hemagglutinin to be able to raise antibody in the presence of maternal antibody. Measles hemagglutinin, fusion and nucleoprotein genes will be expressed in the pGA plasmid. These compositions will, therefore, be suitable for a human vaccine.

### Example 18

### Influenza inserts with and without C3d

Plasmid vector construction and purification procedures have been previously described for JW4303 (Pertmer et al., Vaccine U.13:1427-1430, 1995; Feltquate et al., J. Immunol. 158:2278-2284, 1997). In brief, influenza hemagglutinin (HA) sequences from NPR/8134 (H1N1) were cloned into either the pJW 4303 or pGA eukaryotic expression vector using unique restriction sites.

Two versions of HA, a secreted(s) and a transmembrane (tm) associated, have been previously described (Torres et al., Vaccine 18:805-814, 1999; Feltquate et al., J. Immunol. 158:2278-2284, 1997).

Vectors expressing sHA or tmHA in pJW4303 were designated pJW/sHA and pJW/tmHA respectively and the vectors expressing sHA, tmHA, or sHA-3C3d in pGA were designated pGA5/sHA, pGA3/tmHA, and pGA6/sHA-3C3d respectively. Vectors expressing HA-C3d fusion proteins were generated by cloning three tandem repeats of the mouse homo log of C3d and placing the three tandem repeats in framewith the secreted HA gene. The construct designed was based upon Dempsey et al. (Science 271:348-350, 1996). Linkers composed of two repeats of 4 glycines and a serine were fused at the joints of each C3d repeat. The pGA6/sHA-3C3d plasmid expressed approximately 50% of the protein expressed by the pGA5/sHA vector. However, the ratio of sHA-3C3d found in the supernatant vs. the cell lysate was similar to the ratio of antigen expressed by pGA5/sHA. More than 80% of the protein was secreted into the supernatant. In western analysis, a higher molecular weight band was detected at 120 kDa and represented the sHA-3C3d fusion protein. Therefore, thesHA-3C3d fusion protein is secreted into the supernatant as efficiently as the sHAantigen.

Mice and DNA Immunizations:

Six to 8 week old BALB/c mice (Harlan SpragueDawley, Indianapolis, Ind.) were used for inoculations. Mice, housed in microisolator units and allowed free access to food and water, were cared for under USDA guidelines for laboratory animals. Mice were anesthetized with 0.03-0.04 ml of a mixture of 5 ml ketamine HCl (100 mg/ml) and 1 ml xylazine (20 mg/ml). Gene gun immunizations wereperformed on shaved abdominal skin using the hand held Accell gene delivery system and immunized with two gene gun doses containing 0.5 μg of DNA per 0.5 mg of approximately 1-μm gold beads (DeGussa-Huls Corp., Ridgefield Park, NJ) at a helium pressure setting of 400 psi.

Influenza Virus Challenge:

Challenge with live, mouse-adapted, influenza virus (NPR/8/34) was performed by intranasal instillation of 50 μl allantoic fluid, diluted in PBS to contain 3 lethal doses of virus, into the nares of ketamine-anesthetized mice. This method leads to rapid lung infections and is lethal to 100% of non-immunized mice. Individual mice were challenge at either 8 or 14 weeks after vaccination and monitored for both weight loss and survival. Data were plotted as the average individual weight in a group, as a percentage of pre-challenge weight, versus days after challenge.

Antibody Response to the HA DNA Immunization Protocol:

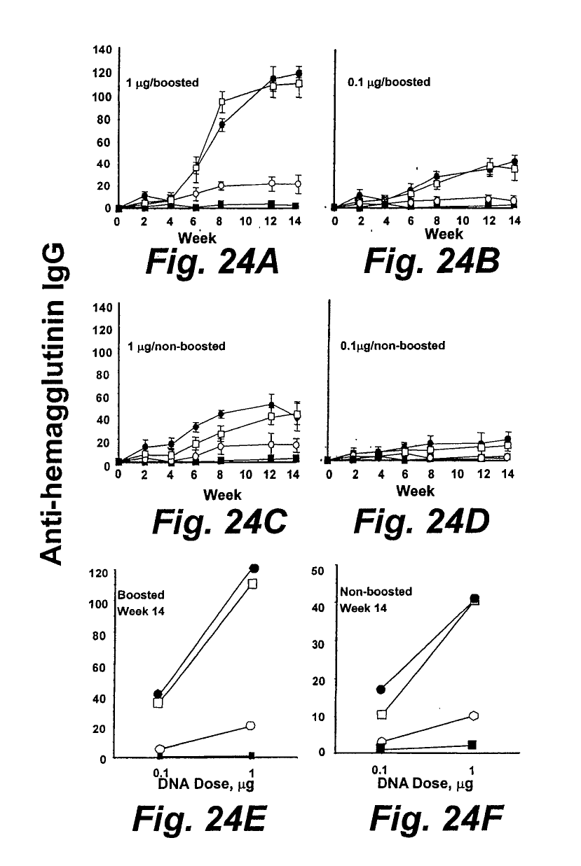

The tmHA and sHA-3C3d expressing DNA plasmids raised higher titers of ELISA antibody than the sHADNA. BALB/c mice were vaccinated by DNA coated gold particles via gene gun with either a 0.1 μg or 1 μg dose inoculum. At 4 weeks post vaccination, half of the mice in each group were boosted with the same dose of DNA given in the first immunization. Total anti-HA IgG induced by the sHA-3C3d- and tmHA-expressing plasmids were similar in the different experimental mouse groups and 3-5 times higher then the amount raised by the sHA expressing plasmids, as shown in FIGS. 24A-24D. In addition, the amount of anti-HA antibody elicited increased relative to the amount of DNA used for vaccination in a dose dependent manner (FIGS. 24E-24F). Overall, the dose response curves and temporal pattern for the appearance of anti-HA antibody were similar in the mice vaccinated with tmHA-DNA or sHA-3C3d-DNA, but lower and slower, in the mice vaccinated with sHA-DNA. As expected, the booster immunization both accelerated and increased the titers of antibodies to HA.

Avidity of Mouse HA Antiserum:

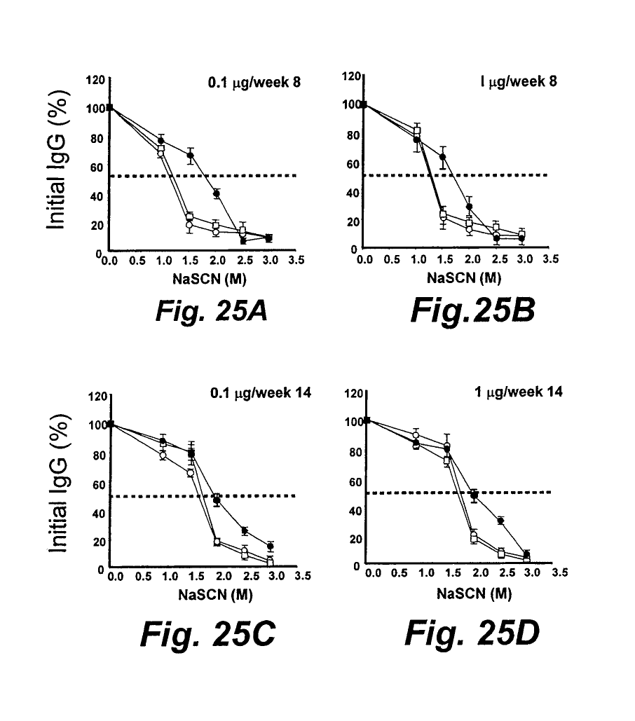

Sodium thiocyanate (NaSCN) displacement ELISAs demonstrated that the avidity of the HA-specific antibody generated with sHA-3C3d expressing DNA was consistently higher than antibodies from sHA-DNA or tmHA-DNA vaccinated mice, as shown in FIGS. 25A-25D. The avidity of specific antibodies to HA was compared by using graded concentrations NaSCN, a chaotropic agent, to disrupt antigen-antibody interactions. The binding of antibodies with less avidity to the antigen is disrupted at lower concentrations of NaSCN than that of antibodies with greater avidity to the antigen. The effective concentration of NaSCN required to release 50% of antiserum (ED50) collected at 8 weeks after vaccination from sHA-DNA or tmHA-DNA boosted mice (0.1 μg dose or 1 μg dose) was approximately 571.20 M (FIG. 25A). In contrast, antiserum from mice vaccinated and boosted with sHA-3C3d-DNA had an ED50 of about 1. 75 M (FIG. 25B). At the time of challenge (14 weeks after vaccination), the ED50 had increased to about 1.8 M for antibodies from both sHADNA and tmHA-DNA vaccinated mice (FIG. 25C). Antibodies from mice vaccinated with sHA-3C3d-DNA had increased to an ED so of about 2.0 M (FIG. 25D). These results suggest that the antibody from sHA-3C3d-DNA vaccinated mice had undergone more rapid affinity maturation than antibody from either sHA-DNA or tmHA-DNA vaccinated mice. The difference between the temporal avidity maturation of antibody for sHA-3C3d and tmHA was independent of the level of the raised antibody. Both of these plasmids had similar temporal patterns for the appearance of antibody and dose response curves for the ability to raise antibody (FIGS. 25A-25D).

Hemagglutinin-Inhibition (HI) Titers:

Hemagglutination-inhibition assays (HI) were performed to evaluate the ability of the raised antibody to block binding of A/PR/8/34 (H1N1) to sialic acid. The HI titers were measured from serum samples harvested from mice at 8 and 14 weeks after vaccination. All boosted mice had measurable HI titers at week 14 regardless of the dose or vaccine given. The highest titers (up to 1:1200) were recorded for the sHA-3C3d-DNA vaccinated mice. Nonboosted mice showed more variation in HI titers. Nonboosted mice vaccinated with a 0.1 μg dose of either sHA-DNA or tmHA-DNA expressing plasmids had low HI titers of 1:10. In contrast, mice vaccinated with sHA-3C3d-DNA had titers greater than 1:640. The only vaccinated mice that had a measurable HI titer (1:160) at week 8 were boosted mice vaccinated with 1 μg dose sHA-3C3d-DNA. These results indicate that C3d, when fused to sHA, is able to stimulate specific B cells to increase the avidity maturation of antibody and thus the production of neutralizing antibodies to HA.

Protective Efficacy to Influenza Challenge:

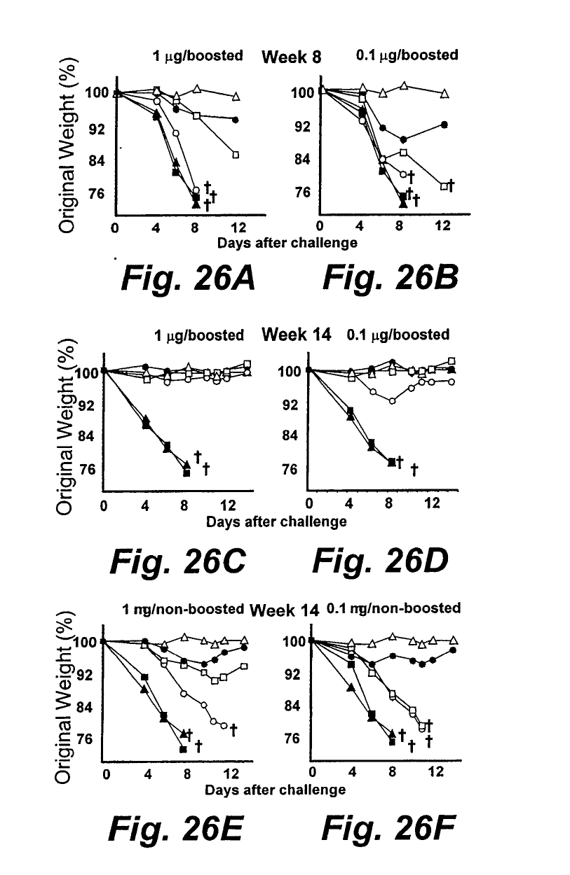

Consistent with Eliciting the Highest titers of HI antibody, the sHA-3C3d DNA raised more effective protection than the sHA or tmHA DNAs. To test the protective efficacy of the various HA-DNA vaccines, mice were challenged with a lethal dose of A/PR/8/34 influenza virus (H1N1) and monitored daily for morbidity (as measured by weight loss) and mortality. Weight loss for each animal was plotted as a percentage of the average pre-challenge weight versus days after challenge, as shown in FIGS. 26A-26F. Virus-challenged naive mice and pGA vector-only vaccinated mice showed rapid weight loss with all the mice losing >20% of their body weight by 8 days post-challenge (FIGS. 26A-26D). In contrast, PBS mice showed no weight loss over the 14 days of observation. All boosted mice survived challenge, 14 weeks after vaccination, regardless of the dose of DNA plasmid administered. However, boosted mice vaccinated with a 0.1 μg dose of sHADNA did drop to 92% of their initial body weight at 8 days post-challenge before recovering (FIG. 26D). In contrast, when 1 μg dose, boosted mice were challenged at 8 weeks after vaccination, the only mice to survive challenge were sHA-3C3d- and tmHADNA vaccinated mice, albeit with greater weight loss than was observed from mice challenged at 14 weeks after vaccination. The only 0.1 μg dose, boosted mice to survive challenge at 8 weeks after vaccination were the sHA-3C3d vaccinated mice (FIG. 26B).

Among the non-boosted, 0.1 μg dose immunizations, only the sHA-3C3d-DNA vaccinated mice survived challenge at 14 weeks after vaccination (FIG. 26F). All mice administered a single DNA vaccination lost weight. However, of these, the sHA-3C3d DNA vaccinated mice lost the least weight and these mice were the only mice to survive the lethal challenge. These results demonstrate the that 3C3d protein, when fused to HA, increased the efficiency of a DNA vaccine, allowing for the reduction in dose of DNA and the number of vaccinations needed to afford protection to a lethal influenza virus challenge.

### Example 19

### HIV gp120-C3d Fusion Constructs

In this study, an approach similar to that described in Example 18 was used to fuse three copies of murine C3d to the carboxyl terminus of HIV Env gp120 subunit. Using DNA vaccination, BALB/c mice were inoculated and assayed for enhanced immune responses. The fusion constructs induced higher antibody responses to Env and a faster onset of avidity maturation than did the respective wild-type gp120 sequences. Thus, the efficacy of DNA vaccines for raising antibody can be significantly improved by fusing proteins with C3d.

Plasmid DNA:

A pGA vaccine vector was constructed as described in Example 1 to contain the cytomegalovirus immediate-early promoter (CMV-IE) plus intron A (IA) for initiating transcription of eukaryotic inserts, and the bovine growth hormone polyadenylation signal (BGH poly A) for termination of transcription. HIV envelope sequences from the isolates HIV-ADA, HIV-IIIB and 89.6, encoding almost the entire gp120 region, and C3d sequences were cloned into the pGA vaccine vector using unique restriction endonuclease sites. The gp120 segment encoded a region from amino acid 32 to amino acid 465 and ended with the amino acid sequence V APTRA (SEQ ID NO: ______). The first 32 amino acids were deleted from the N-terminus of each sgp 120 and replaced with a leader sequenced from the tissue plasminogen activator (tpA). The vectors expressing sgp120-C3d fusion proteins were generated by cloning three tandem repeats of the mouse homologue of C3d in frame with the sgp120 expressing DNA. The construct design was based upon Dempsey et al. (Science 271:348-350, 1996). Linkers composed of two repeats of four glycine residues and a serine were fused at the junctures of HA and C3d and between each C3d repeat. Potential proteolytic cleavage sites between the junctions of C3d and the junction of 3C3d were mutated by ligating Barn HI and Bgl II restriction endonuclease sites to mutate an Arg codon to a Gly codon.

The plasmids were amplified in Escherichia coli strain-DH5a, purified using anion-exchange resin columns (Qiagen, Valencia, Calif.) and stored at −20° C. in dH20. Plasmids were verified by appropriate restriction enzyme digestion and gel electrophoresis. Purity of DNA preparations was determined by optical density reading at 260 nm and 280 nm.

Mice and DNA Immunizations:

Six to 8 week old BALB/c mice (Harlan Sprague Dawley, Indianapolis, Ind.) were vaccinated. Briefly, mice were immunized with two gene gun doses containing 0.5 μg of DNA per 0.5 mg of approximately 1 μm gold beads (DeGussa-Huls Corp., Ridgefield Park, NJ) at a helium pressure setting of 400 psi. The human embryonic kidney cell line 293T (5×10⁵ cells/transfection) was transfected with 2 μg of DNA using 12% lipofectamine according to the manufacturer's guidelines (Life Technologies, Grand Island, N.Y.). Supernatants were collected and stored at −20° C. Quantitative antigen capture ELISAs for H were conducted as previously described (Cardoso et al., Virology 225:293-299, 1998).

For western hybridization analysis, 15 μl of supernatant or cell lysate was diluted 1:2 in SDS sample buffer (Bio-Rad, Hercules, Calif.) and loaded onto a 10% polyacrylamide/SDS gel. The resolved proteins were transferred onto a nitrocellulose membrane (Bio-Rad, Hercules, Calif.) and incubated with a 1:1000 dilution of polyclonal human HIV-infected patient antisera in PBS containing 0.1% Tween 20 and 1% nonfat dry milk. After extensive washing, bound rabbit antibodies were detected using a 1:2000 dilution of horseradish peroxidase-conjugated goat anti-rabbit antiserum and enhanced chemiluminescence (Amersham, Buckinghamshire, UK).

ELISA and Avidity Assays:

An end point ELISA was performed to assess the titers of anti-Env 1gG in immune serum using purified HIV-1-IIIB gp120 CHO-expressed protein (Intracell) to coat plates as described (Richmond et al., J. Viral. 72:9092-9100,1998). Alternatively, plates were coated with sheep anti-Env antibody (International Enzymes Inc., Fallbrook, Calif.) and used to capture sgp120 produced in 293T cells that were transiently transfected with sgp120 expression vectors. Mouse sera from vaccinated mice was allowed to bind and subsequently detected by anti-mouse IgG conjugated to horseradish peroxidase. Endpoint titers were considered positive that were two-fold higher than background. Avidity ELISAs were performed similarly to serum antibody determination ELISAs up to the addition of samples and standards. Samples were diluted to give similar concentrations of specific IgG as determined by O.D. measurements. Plates were washed three times with 0.05% PBS-Tween 20. Different concentrations of the chaotropic agent sodium thiocyanate (NaSCN), in PBS (0 M, 1M, 1.5 M, 2 M, 2.5 M, and 3M NaSCN), were then added. Plates were allowed to stand at room temperature for 15 minutes and then washed six times with PBS-Tween 20. Subsequent steps were performed similarly to the serum antibody determination ELISA and percent of initial IgG calculated as a percent of the initial O.D. All assays were done in triplicate. Neutralizing antibody assays: Antibody-mediated neutralization of HIV-1-IIIB and 89.6 was measured in an MT-2 cell-killing assay as described previously (Montefiori et al., J. Clin. Microbial. 26:231-237, 1988). Briefly, cell-free virus (50 pl containing 10⁸ TCID₅₀ of virus) was added to multiple dilutions of serum samples in 100 pl of growth medium in triplicate wells of 96-well microtiter plates coated with poly-L-lysine and incubated at 37° C. for one hour before MT-2 cells were added (105 cells in 100 pi added per well). Cell densities were reduced and the medium was replaced after 3 days of incubation when necessary. Neutralization was measured by staining viable cells with Pinter's neutral red when cytopathic effects in control wells were >70% but less than 100%. The percentage protection was determined by calculating the difference in absorption (As40) between test wells (cells+ virus) and dividing this result by the difference in absorption between cell control wells (cells only) and virus control wells (virus only). Neutralizing titers are expressed as the reciprocal of the plasma dilution required to protect at least 50% of cells from virus-induced killing.

Results:

Env was expressed at overall similar levels by plasmids containing either the secreted form of the antigen, but at a two-four-fold lower level by the sgp120-C3d expressing plasmids. Human 293T cells were transiently transfected with 2 μg of plasmid and both supernatants and cell lysates were assayed for gp120 using an antigen capture ELISA. The sgp120 constructs expressed from 450 to 800 ng per ml, whereas the 3C3d fusions expressed from 140 to 250 ng per ml. Approximately 90% of the Env protein was present in the supernatant for both sgp120 and sgp120-3C3d-DNA transfected cells. The approximately 2-fold differences in the levels of expression of the different sgp120s is likely to reflect differences in the Env genes as well as differences in the efficiency that the capture and detection antibodies recognized the different Envs.

Western blot analyses revealed sgp120 and sgp120-3C3d proteins of the expected sizes. Using human patient polyclonal antisera, Western blot analysis showed the expected broad band of 115-120 kD corresponding to gp120. A higher molecular weight band at about 240 kD was consistent with the projected size of the sgp 120-3C3d fusion protein. Consistent with the antigen-capture assay, intense protein bands were present in the supernatants of cells transfected with sgp120-DNA, whereas less intense bands were present in the supernatants of cells transfected with sgp120-3C3d-DNA. No evidence for the proteolytic cleavage of the sgp120-C3d fusion protein was seen by Western analysis.

Antibody Response to Env Gp120 DNA Immunizations:

The sgp 120-3C3d expressing DNA plasmids raised higher titers of ELISA antibody than the sgp120 DNA.BALB/c mice were vaccinated by DNA coated gold particles via gene gun with a 1 μg dose inoculum. Mice were vaccinated at day 1 and then boosted at 4, 14, and 26 weeks with the same DNA given in the first immunization. When sera were assayed on gp120-IIIB-coated plates, mice vaccinated with the DNAs expressing the C3d fusion proteins had anti-Env antibodies 3-7 times higher then the amount of antibody raised by the counterpart sgp120 expressing plasmids. Among the C3d constructs, mice vaccinated with sgp120-(IIIB)-3C3d had the highest levels of antibody and mice vaccinated with sgp 120-(ADA)-3C3d expressing DNA had the lowest levels of anti-Env antibodies. The temporal pattern for the appearance of anti-Env antibody revealed titers being boosted at each of the inoculations for all constructs tested.

Differences in the levels of the antibody raised by the different Envs appeared to be determined by the specificity of the raised antibody. Using an alternative ELISA protocol, in which antibody was captured on the homologous Env, all of the C3d-fusions appeared to raise similar levels of antibody. In this assay, sheep anti-Env antibody was used to capture transiently produced sgp 120 proteins. This assay revealed low, but similar levels of antibody raised by each of the sgp120-3C3d constructs. The lower levels of antibody detected in this assay are likely to reflect the levels of transfection produced Env used to capture antibody being lower than in the assays using commercially produced IIIB gp120 to coat plates. As expected using either ELISA method, booster immunizations were necessary to achieve even the most modest antibody response.

Avidity of Mouse Env Antiserum:

Sodium thiocyanate (NaSCN) displacement ELISAs demonstrated that the avidity of the antibody generated with sgp120-3C3d expressing DNA was consistently higher than that from sgp 120-DNA vaccinated mice. Avidity assays were conducted on sera raised by sgp120-(IIIB) and sgp120-(IIIB)-3C3d because of the type specificity of the raised antisera and the commercial availability of the IIIB protein (but not the other proteins) for use as capture antigen. The avidity of specific antibodies to Env was compared by using graded concentrations NaSCN, a chaotropic agent, to disrupt antigen-antibody interaction. Results indicated that the antibody from sgp120-3C3d-DNA vaccinated mice underwent more rapid affinity maturation than antibody from sgp 120-DNA vaccinated mice.

Env-3C3d Expressing Plasmids Elicit Modest Neutralizing Antibody:

Neutralizing antibody studies performed on MT-2 cells detected higher titers of neutralizing activity in the sera generated by the gp120-3C3d constructs than in the sera generated by the sgp120 constructs. Sera were tested against two syncytium-inducing, IIIB (X4) and 89.6 (X4R5) viruses. Mice vaccinated with sgp 120-3C3d expressing plasmids had very modest levels of neutralizing antibody to the homologous strain of HIV tested by the protection of MT-2 cells from virus-induced killing as measured by neutral red uptake. Titers of neutralizing antibody raised by the gp 120-expressing DNAs were at the background of the assay.

The results of this study showed that fusions of HIV-1 Env to three copies of murine C3d enhanced the antibody response to Env in vaccinated mice. Mice vaccinated with any of the three DNA plasmids expressing sgp120 sequence had low or undetectable levels of antibody after 4 vaccinations (28 weeks post-prime).

In contrast, mice vaccinated with DNA expressing the fusion of sgp 120 and 3C3d proteins elicited a faster onset of antibody (3 vaccinations), as well as higher levels of antibodies. In contrast to the enhancement of antibody titers and avidity maturation of antibodies to Env, the amount of neutralizing antibody elicited in the vaccinated mice was low. Mice vaccinated with plasmids expressing sgp120 had low levels of neutralizing antibody that were only modestly increased in mice vaccinated with sgp 120-3C3d expressing plasmids. However, the levels of neutralizing antibodies did apparently increase after the fourth immunization. The poor titers of neutralizing antibody could have reflected an inherent poor ability of the sgp120-3C3d fusion protein to raise neutralizing antibody because of the failure to adequately expose neutralizing epitopes to responding B cells. The intrinsic high backgrounds for HIV-1 neutralization assays in mouse sera also may have contributed to the poor neutralization titers.

The results demonstrate the effectiveness of C3d-fusions as a molecular adjuvant in enhancing antibody production and enhancing antibody maturation. In addition, the neutralizing antibody response to Env was modestly increased in mice vaccinated with C3d-fusion vaccines. Similar to results seen in Example 18, using secreted versions of HA from the influenza virus, C3d-enhanced antibody responses were achieved with plasmids expressing only half as much protein as plasmids expressing non-fused sgp 120.

### Example 20

### An MVA “only” vaccine

The studies that follow were conducted to evaluate the ability of the MVA component of a vaccine to serve as both a prime and a boost (in, for example, an AIDS or smallpox vaccine). The same immunization schedule, MV A dose, and challenge conditions are used as in the DNA/MVA vaccine trial described above. As shown below, the MVA-only vaccine raised less than one-tenth of the number of vaccine-specific T cells but ten-times higher titers of binding antibody for Env than the DNA/MVA-vaccine. Post challenge, the MVA-only vaccinated animals expanded their CD8 cells to levels that were similar to those in DNA/MVA vaccinated animals. However, they underwent a slower emergence and contraction of anti-viral CDS T cells and were slower to generate neutralizing antibodies than the DNA/MVA vaccinated animals. Despite this, by 5 weeks post challenge, the MVA-only vaccinated animals had achieved a level of control of the viral infection that was as good as that seen in the DNA/MVA group, a situation that has held up to the current time in the trial (48 weeks post challenge).

Immunogens, immunizations and challenge: Immunogens were constructed and produced as described in Amara et al. (Science 292:69-74, 2001; see also, above). Young adult rhesus macaques from the Yerkes breeding colony were cared for under guidelines established by the Animal Welfare Act and the NIH “Guide for the Care and Use of Laboratory Animals” using protocols approved by the Emory University Institutional Animal Care and Use Committee. Macaques were typed for the Mamu-A*01 allele using PCR analyses (Knapp et al., Tissue Antigens 50:657-661, 1997). The DNA/MVA group used as an example of DNA/MVA immunizations received 2.5 mg of DNA intradermally at 0 and 8 weeks and MVA at 24 weeks (group 1 in Amara et al., and as above). Recombinant MVA immunizations were administered both intradermally and intramuscularly with a needle for a total dose of 2×10⁸ pfu as previously described at 0,8, and 24 weeks. Control animals received vector DNA as well as MVA without inserts at 0, 8 and 14 weeks (Amara et al., Science 292:69-74, 2001). Seven months after the rMVA booster, animals received an intrarectal challenge with SHIV-89.6P using a pediatric feeding tube to introduce 20 intrarectal infectious units (1.2×10¹⁰ copies of SHIV89.6P viral RNA) 15 to 20 cm into the rectum. Animal numbers are as follows: 1,RBr-5*; 2, RIm-5*; 3, RQf-5*; 4, RZe-5; 5, ROm-5; 6, RDm-5; 25, RMb-5*; 26, RGy-5*; 27, RUs-4; 28, RPm-5; 29, RPs-4; 30, RKj-5; 43, RMr-4*; 44, RZt-4*; 45, RPk-5; 46, RRk-5; 47, RK1-5; 48, RGh-5. Rhesus with the A*01 allele are indicated with asterisks.

T cell responses: For tetramer analyses, approximately 1×10⁶ PBMC were surface stained with antibodies to CD3 (FN-18, Biosource International, Camarillo, Calif.),65CDS (SK1, Becton Dickinson, San Jose, Calif.), and Gag-CM9 (CTPYDINQM)-Mamu-A *O 1 tetramer conjugated to different fluorochromes (for details, see Amara et al., and the Examples above). For IFN-γ ELISPOTs, anti-human IFN-γ antibody (Clone B27, Pharmingen, San Diego, Calif.) was used for capture and biotinylated anti-human IFN-γ antibody (clone 7-B6-1, Diapharma Group Inc., West Chester, Ohio) followed by AvidinHRP (Vector Laboratories Inc, Burlingame, Calif.) for detection. The frequencies of ELISPOTs are approximate because different dilutions of cells have different efficiencies of spot formation in the absence of feeder layers (Power et al., J. Immunol. Methods 227:99-107, 1999).

Quantitation of SHIV Copy Number:

SHIV copy number was determined using a quantitative real time PCR as described by Amara et al. (Science 292:69-74, 2001) and Hofinann-Lehmann et al. (AIDS Res. Hum. Retroviruses 16:1247-1257, 2000). All specimens were extracted and amplified in duplicate, with the mean result reported.

Intracellular p27 Staining:

Approximately 1×10⁶ PBMC were fixed and permeabilized with Cytofix/Cytoperm solution (Pharmingen, Inc.), and stained sequentially with anti-SIV gag Ab (clone F A-2, obtained from NIH AIDS reagent program) and PE-conjugated anti-mouse 1 g (Pharmingen, Inc.) in perm wash for 30 minutes at 4° C. Cells were washed twice with perm wash and incubated with antibodies to human CD3 (clone FN-18, Bio source International, Camarillo, Calif.) and CDS (clone SK1, Becton Dickinson) conjugated to FITC and PerCP respectively in Permwash solution. Approximately 150,000 lymphocytes were acquired on the FACScaliber and analyzed using FloJo™ software

Gag and Env ELISAs:

ELISAs for total anti-Gag antibody and anti-Env antibody were carried out as described by Amara et al. (Science 292:69-74, 2001; and see above). Standard curves for Gag and Env ELISAs were produced using serum from a SHIV-89.6-infected macaque with known amounts of anti-Gag or anti-Env IgG. Sera were assayed at 3-fold dilutions in duplicate wells. Standard curves were fitted and sample concentrations were interpolated as μg of antibody per ml of serum using SOFTmax™2.3 software (Molecular Devices, Sunnyvale, Calif.). Avidity of the Env-specific antibodies was measured using NaSCN displacement ELISAs as described by Amara et al. (Science 292:69-74, 2001; and see above). Briefly, plates were coated overnight with 0.5 μg per ml of recombinant gp120 89.6. The remaining steps were similar to that of anti-Env ELISAs except for an incubation (15 minutes) with different concentrations of NaSCN prior to the addition of anti-monkey 1 gG-HRP conjugate. All samples were assayed in duplicate over a range of dilutions, and results were expressed as the percentage of antibody bound in the absence of NaSCN.

Statistical Analysis:

To examine the effect of dose and immunogen over time on parameters such as viral load, CD4 level, antibody and T cell responses, linear mixed effects models were applied to log-transformed values (Pinheiro and Bates, Mixed Effects Models in Sand S-PLUS, Springer, New York, N.Y.). In these analyses, a difference in the level of a parameter for different groups was indicated by a significant main effect. A difference in the rate of change over time (slope) of a parameter for different groups was indicated by a significant group x week interaction. For determining differences in a parameter at a specific time, the t-test was performed on log-transformed values.

Results:

The MVA vaccine expressed SIV mac239 Gag-Pol and SHIV-89.6 Env within a single recombinant MV A termed MV A/89.6 (Amara et al., Science 292:69-74, 2001). Inoculations of 2×10⁸ pfu of MV A/89.6, one half administered intramuscularly and one half intradermally, were given at 0, 8, and 24 weeks. For the DNA/MVA vaccine, various doses of a Gag-Pol-Env expressing DNA (DNA/89.6) were administered at 0 and 8 weeks and the 2×10⁸ pfu of MV A/89.6 at 24 weeks (Amara et al., Science 292:69-74, 2001). For comparisons with the MVA-only group, we present data from the DNA/MVA group with the highest T cell responses. This group was primed with 2.5 mg of DNA/89.6 intradermally. An intrarectal challenge with SHIV-89.6P was administered at seven months after the final immunization. The 89.6 immunogen and the 89.6P challenge virus do not raise cross-neutralizing activity early after infection (Montefiori et al., J Viral. 72:3427-3431, 1998). Thus, the choice of immunogen and challenge approached the real world situation in which an HIV-1 immunogen is unlikely to raise neutralizing antibody for the challenge virus.

Different Patterns of Vaccine Raised Responses.

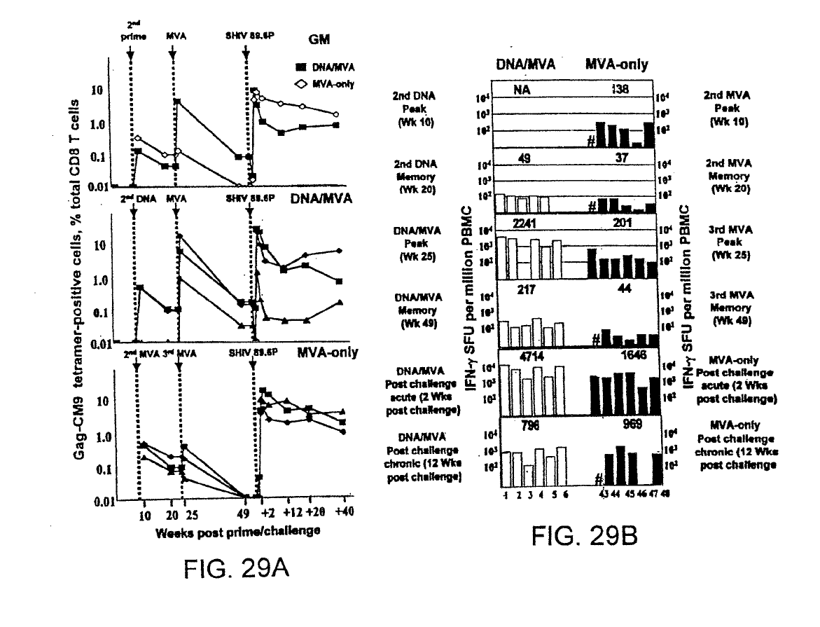

Much lower frequencies of Gag specific T cells were raised in the MVA-only than in the DNA/MVA-vaccinated macaques (FIGS. 29A and 29B). The frequencies of responding T cells were measured using Gag-CM9 tetramer-analyses (Allen et al., J Immunol. 164:4968-4978, 2000) and pools of overlapping Gag peptides and an enzyme linked immunospot (ELISPOT) assay (Kem et al., J. Virol. 73:8179-8184, 1999; Power et al., J. Immunol. Methods 227:99-107, 1999). The tetrarner analyses were restricted to macaques that expressed the MamuA*01 histocompatibility type, whereas ELISPOT responses did not depend on a specific histocompatibility type. Two weeks after the second MV A inoculation, the frequencies of CD8 cells for the Gag-CM9 epitope in A *01 macaques had a geometric mean frequency of 0.35%, which was slightly higher than had been achieved post DNA priming in the DNA/MVA group (FIG. 29A). The third MVA inoculation did not further boost the CD8 response in the MVA-only group. This was in sharp contrast to the DNA/MVA-vaccinated animals, where the MVA booster increased the frequency of tetramer-specific cells by 60 to 200 fold, achieving frequencies as high as 22% of total CD8+ T cells. These frequencies were at least 20 times higher than those observed in MVA-only vaccinated animals at any time prior to SHN challenge. A similar temporal pattern of T cell responses was observed using IFN-γ-ELISPOT analyses (FIG. 29B). In these analyses, DNA/MVA-vaccinated animals had 10 times higher frequencies of IFN-γ producing cells following the MVA booster than the MVA-only group (P=0.001, t test). At the time of challenge IFN-γ ELISPOTs had contracted into memory and were barely detectable in MVA-vaccinated animals as compared with geometric mean frequencies of 217 in the DNA/MVA-vaccinated group (P=0.009, t test).

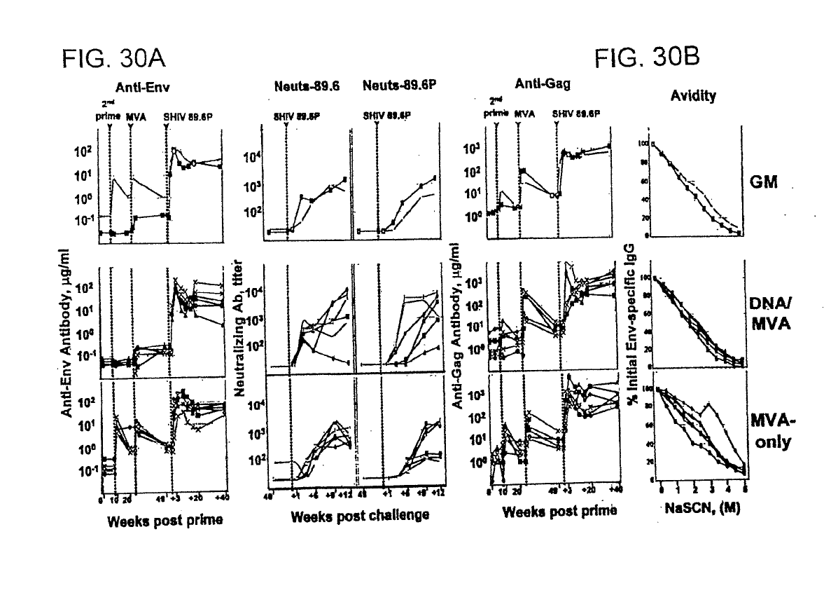

In contrast to the T cell responses, vaccine-raised antibody responses to Env were much higher in the MV A-only than in the DNA/MV A-group (FIGS. 30A and 30B). The second MVA immunization raised good titers of binding antibodies for both Env and Gag (˜10 and 30 μg of specific antibody per ml of serum respectively). These titers were only marginally increased by the third MVA immunization (FIGS. 30A and 30B). In contrast, the DNA/MVA immunizations raised very low levels of anti-Env binding antibody that could be detected only after the MVA booster (FIGS. 30A and 30B). Following the MVA booster, the DNA/MVA animals had good titers of anti-Gag antibody, slightly higher than in the MVA-only animals (FIGS. 30A and 30B). These differences were not significant (t test). Prior to challenge, none of the groups scored for neutralizing antibodies to SHN-89.6 or SHN-89.6P (FIGS. 30A and 30B).

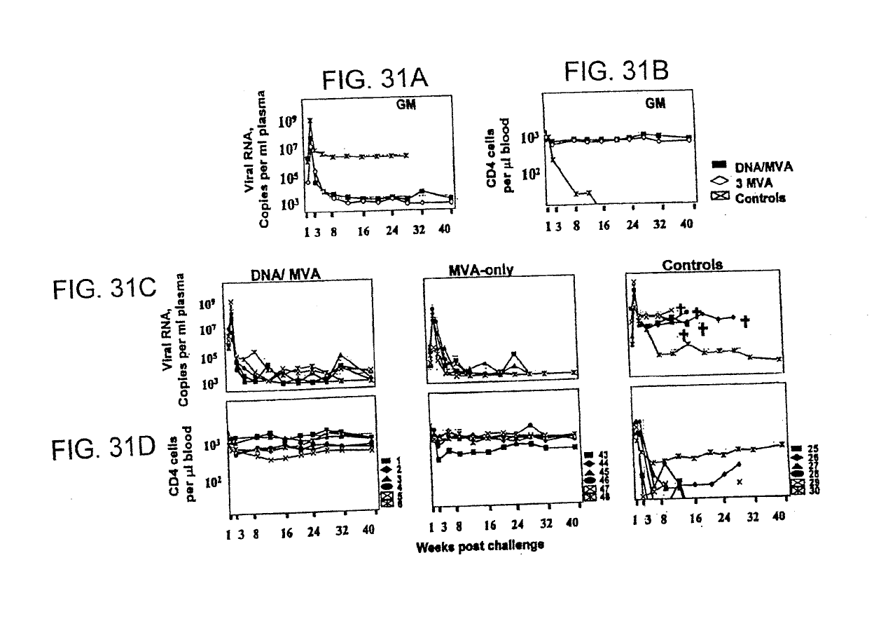

Comparable control of the SHIV 89.6P challenge. All six of the MVA-vaccinated animals controlled their post challenge infections to the limit of detection and protected their CD4 cells (FIG. 31A-31D). At two weeks post challenge, the geometric mean for the peak titers of plasma viral RNA in the MVA-only vaccinated animals (5×10⁶) was about 4 times less than that in DNA/MVA-vaccinated animals (2×10⁷) and 100 times less than that in control animals (3.3×108) (FIG. 31A). The rate and magnitude of virus control between weeks two and three post-challenge in the MVA-only vaccinated animals was slower than in the DNA/MVA-vaccinated animals. These differences between the two vaccine groups did not reach statistical significance. By 5 weeks postchallenge the two groups had similar levels of viremia (FIGS. 31A, 31C). By 40 weekspost challenge, five out of the six control animals had succumbed to AIDS, whereas all of the MVA-only as well as all of the DNA/MVA-vaccinated animals were healthy and maintaining their plasma viral RNA levels at or below the level of detection (FIGS. 31A, 31C).

Slower kinetics of T cell expansion and contraction. Interestingly, the control of the viral challenge in the MVA-only vaccinated animals was associated with both a slower expansion and contraction of the anti-viral T cell response than in the DNA/MVA-vaccinated animals (FIG. 29A). In contrast to the DNA/MVA animals, where the peak expansion of tetramer-positive cells in peripheral blood was observed two weeks post challenge, in the MVA-only animals, the peak expansion occurred three weeks post challenge. Frequencies at the peak response were very similar in the two groups (geometric means of ˜10% of total CD8 cells). The frequencies of IFN-γ ELISPOTs at two weeks post challenge were consistent with this slower expansion (1646 spots per million PBMC in the MVA-only group as opposed to 4714 spots permillion PBMC in the DNA/MVA group) (FIG. 29B). Provocatively, the decline of the tetramer-specific CDS+ cells between weeks 2 and 5 in the MV A-only animals was significantly slower than in the DNA/MVA vaccinated animals (P=0.01, linear mixed effects model) (FIG. 29A). At 12 weeks post challenge, both groups had controlled their levels of plasma viral RNA to similar levels. However, tetramer-positive cells had fallen by only 2-fold in the MVA-only group as opposed to 10-fold in the DNA/MVA group. This slow contraction in the MVA-only group has continued out to the current time in the trial (48 weeks). The slow contraction was also evident in the IFN-γ ELISPOT response (FIG. 29B); by 12 weeks post challenge, the geometric mean frequencies of IFN-γ ELISPOTs in MVA-vaccinated animals had fallen less than 2-fold (from 1646 to 969),whereas in DNA/MVA vaccinated animals ELISPOT frequencies had fallen 6-fold (4714 to 796).

Slower emergence of anti-Env antibody. Despite the priming of much higher titers of binding antibody for Env in the MVA-only group, binding antibodies as well as measurable neutralizing antibodies for both 89.6 and 89.6P emerged more slowly in this group than in the DNA/MVA group (FIG. 30A). Binding antibodies for Env peaked at 5 to 9 weeks post challenge in MVA-only animals, whereas they had peaked by 5 weeks post challenge in DNA/MVA vaccinated animals. The appearance of neutralizing antibodies also was about 4 weeks slower for both 89.6 and 89.6P in the MVA-only than in the DNA/MVA-vaccinated animals. The titers of binding and neutralizing antibody for 89.6 reached similar heights in both groups. However, the titers of neutralizing antibody for 89.6P remained about 5-fold lower in the MVA-only group than in the DNA/MVA group for as long as 12 weeks post challenge. The slower appearance of neutralizing antibodies in the MVA-only animals was not due to differences in the avidity of the binding antibody; indeed, MV A vaccinated animals had slightly higher avidity antibody compared to DNA/MVA vaccinated animals (FIG. 30B). As with the T cell responses, the contraction of the binding antibody response between 5 and 12 weeks postchallenge was significantly slower in the MVA-only than in the DNA/MVA vaccinated group (P=0.01, linear mixed-effects model)(FIG. 3A). Post challenge, both groups had similar anamnestic responses to Gag that peaked at close to 1 mg of anti-Gag antibody per ml of serum (FIG. 31A).

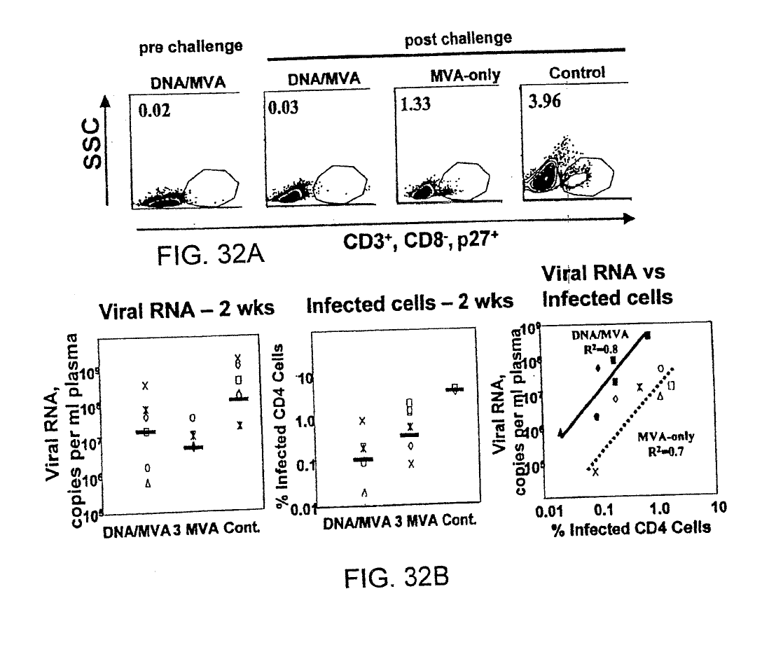

Despite lower levels of plasma viral RNA, the frequencies of infected CD4 cells were higher in the MVA-only than in the DNA-vaccinated group (FIGS. 32A and 32B). At two weeks post challenge, the frequencies of infected cells had a geometric mean of 4% in the control group, 0.5% in the MVA-only group, and 0.1% in the DNA/MVA group. Similar frequencies were observed in lymph nodes. These frequencies and differences in frequencies were also seen in co-cultivation assays. This rank order did not agree with the rank order for the geometric mean titers for plasma viral RNA where the MVA-vaccinated group had the lowest value (FIG. 32B). When levels of plasma viral RNA were compared to levels of infected cells in the DNA/MVA and MVA-only groups, both showed direct but distinct correlations (FIG. 32B, third panel). Presumably this reflected the differences in the antiviral T cell and antibody responses in these two sets of animals.

The MVA-only vaccine controlled plasma viremia and protected CD4+ cells as a DNA/MVA vaccine (FIGS. 31A-31D). Despite similar viral control and CD4 protection, patterns of immune responses in the two vaccine groups were strikingly different before challenge (FIGS. 29A, 29B, 30A, and 30B). During the immunization phase of the trial, priming of anti-Env antibody was much higher for the MVA-only group, whereas MVA priming of MVA T cells was much higher for the DNA/MVA group. Seven months after the MVA final immunization, at the time of challenge, the MVA-only group had undetectable levels of specific T cells whereas the DNA/MVA group had easily detected levels in the peripheral blood. At the same time the MVA-group had 10-times higher levels of binding antibody for Env than the DNA/MVA group. Surprisingly, these differences in vaccine-raised responses did not have major effects on post challenge anamnestic responses and viral control, which were similar except for slower kinetics in the MVA only group (FIGS. 29A, 29B, 30A, and 30B). Immune cell trafficking may have been different in MVA-only and DNA/MVA groups. This could have accounted for the slower kinetics of post challenge T cell as well as neutralizing antibody responses in the peripheral blood for the MVA-only group.

A notable difference between the two immunization paradigms has been the slower contraction of immune responses in the MV A-only-treated animals. Even 48 weeks post challenge, both humoral and cellular responses remain higher in the MVA only group than in the DNA-MVA group (FIGS. 29A, 29B, 30A, and 30B). This phenomenon occurred despite viremia being even more tightly controlled between 12 and 24 weeks post challenge in the MVA-only group than in the DNA/MVA group (P=0.02, linear mixed-effects model). The higher levels of persisting immune responses in the MVA-only group could be a marker for higher levels of sequestered and persisting virus in this group.

This trial achieved better and more consistent protection than has been achieved in prior MVA-only trials (Barouch et al., J. Viral. 75:5151-5158, 2001; Ourmanov et al., J. Viral. 74:2740-2751, 2000). A factor contributing to this difference may have been the use of an intrarectal challenge. The intrarectal, as opposed to an intravenous challenge, allows the immune system added time to respond to an infection that is at least transiently sequestered in the gut (Benson et al., J. Viral. 72:4170-4182, 1998). An intrarectal challenge is also relevant to the current AIDS pandemic in which the vast majority of infections are spread by mucosal routes during sexual intercourse. Another potentially important difference between this trial and the less protective trial using SIVSmE660 was the much slower appearance of neutralizing antibodies following challenge with E660 virus (Ourmanov et al., J. Viral. 74:2960-2965, 2000). Differences in the virulence of SIVsmE660 and SHIV-89.6P also could have contributed to the present success.

The success of the MVA-only vaccine, despite its not having raised the highest T cell responses, highlights the importance of testing for protective efficacy as well as immunogenicity during vaccine development. These results demonstrate that different vaccine modalities can have similar post-challenge control of infection despite very different patterns of pre-challenge immune responses.

### Example 21

### Vaccination Against Smallpox

One of the possible limitations of live-vectored vaccines is pre-existing immunity to the vector. About 45% of the U.S. population currently has neutralizing antibodies against adenovirus. Older people, who were vaccinated for smallpox, will have preexisting immunity for MVA; an immunity that would become universal if vaccinations for smallpox became routine to counter the threat of bioterrorism. However, rMVA vaccines can serve a dual purpose: immunization against smallpox as well as HIV-1. The dual vaccine would have the practical as well as cost advantages of achieving two immunizations with one vaccine and could provide a smallpox vaccine with a lower incidence of adverse events than the current vaccine. Pre-existing immunity can be overcome by higher doses of vaccines and by heterologous prime/boost protocols. Higher doses of vaccine represent a brute force approach to immunizing in the presence of pre-existing immunity. Priming with an agent for which there is not pre-existing immunity, such as DNA, establishes memory cells that require the booster to achieve only sufficient infection to augment the primed immune response. Nevertheless, for both r MVA and Ad5 vaccines, a vector-naive population is the simplest and preferred population for vaccination.

Comparative Immunogenicity of MVA and MVAIHIV-1-48:

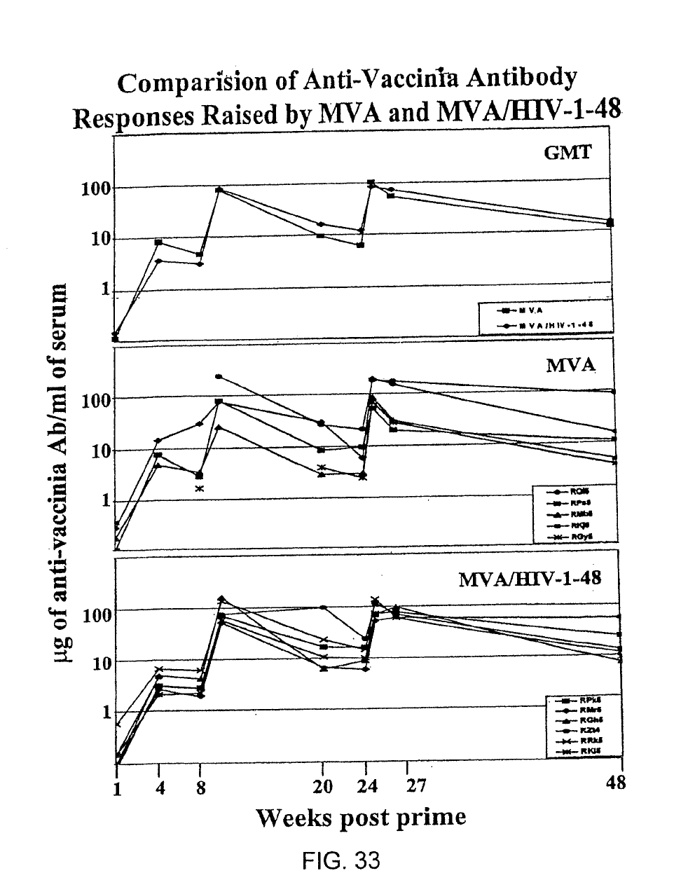

In a pre-clinical trial in macaques, MVA and MVA/HIV-1-48 were found to raise similar titers of antivaccinia antibody. The ability of MV A and MVA/HIV-1-48 to raise antibody to vaccinia, were compared in macaques that had been inoculated with 2×10⁸ pfu of the MVA respective MVA viruses at 0, 8 and 24 weeks. One half of the inoculum was delivered intradermally and the second half was delivered intramuscularly. Sera were harvested a t0, 4, 8, 10, 20, 24, 25, and 27 days and assayed for antibody to vaccinia virus using an ELISA (see the method described below) (known amounts of macaque IgG was used as a standard). The results of these assays revealed that the recombinant MVA raised indistinguishable titers of anti-vaccinia antibody from the wild type MVA. FIG. 33 (uppermost panel) shows the geometric mean titers (GMT) for antibody raised by recombinant and wild type MVA; the middle panel shows the titers for anti-vaccinia antibody for the five individual monkeys used to test the wild type MVA for the ability to raise anti-vaccinia antibody; and the lower panel shows the titers of vaccinia virus antibody for the six individual macaques used to test the MVA/HIV-48 for the ability to raise anti-vaccinia antibody.

ELISA:

The materials required include bicarbonate buffer, WR stock, titer 2×10¹⁰ dilution buffer, 4% whey buffer, 2% paraformaldehyde (recommended storage at 4° C.), goat anti-monkey IgG-UNLB (stock at 10 mg/ml), Rhesus monkey IgG (stock at 5 mg/ml), goat anti-monkey IgG-PO, phosphate/citrate buffer, TMB substrate tablets, and 4N H₂SO₄.

On Day One:
Coat the first vertical columns of each plate with Goat anti-monkey IgG-UNLB at 4 ug/ml in bicarbonate buffer for standard use;
Coat the rest of the plate with WR Vaccinia stock at 0.5 ul/ml in bicarbonate buffer;
Incubate the plates in 37c 5% CO2 incubator over night.

On Day Two: 73
Pour off the liquid, and fill the first two columns of the plate with dilution buffer;
Put 100 μl of 2% parafonnaldehyde per well to the rest of the well, which were coated with Vaccinia stock;
Incubate 10 minutes at 4° C.;
Wash the plates in 1×PBS Triton X-100, 3 times;
Block the plates with 5% milk in dilution buffer for 1 hour at room temperature;
Repeat wash 3 times.
Prepare the samples by:
Diluting the standard Rhesus monkey IgG with dilution to 100 ng/ml, 200 μl per well for the first well of the first 2 columns (perform 2 fold serial dilution vertically);
Dilute the samples at desired dilution, perform serial dilution if necessary;•Incubate the plates at room temperature for 1 hour;
Wash 3 times.
Make goat anti-monkey IgG-PO at 1:4000 in dilution buffer, 100 μl per well, 1 hour incubation at room temperature;
Wash 3 times
Add TMB tablets in phosphate/citrate buffer, 100 μl per well, let develop for 5-15 minutes;
Stop the reaction by adding 4N H2S04 25 μl per well;
Read plates at 450 nm.

### Example 22

### Clade AG Vaccine Inserts

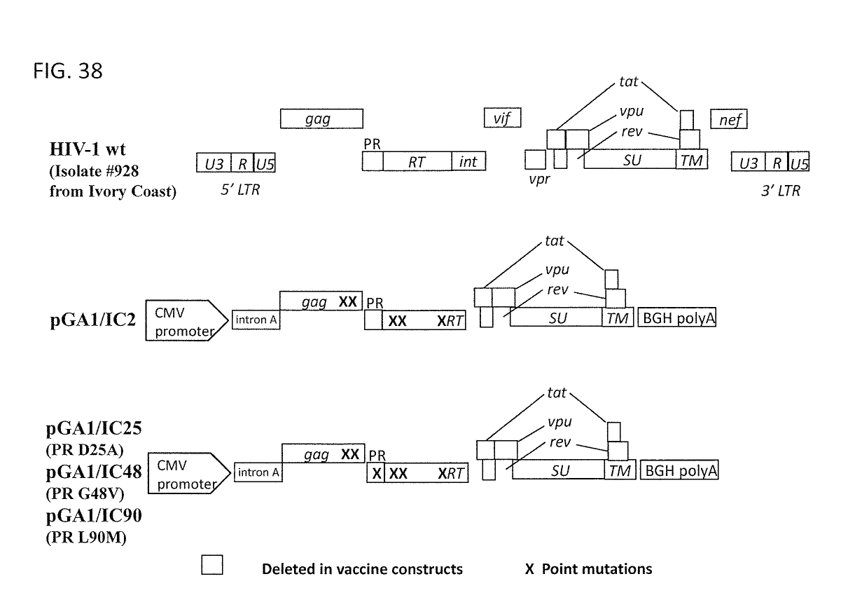

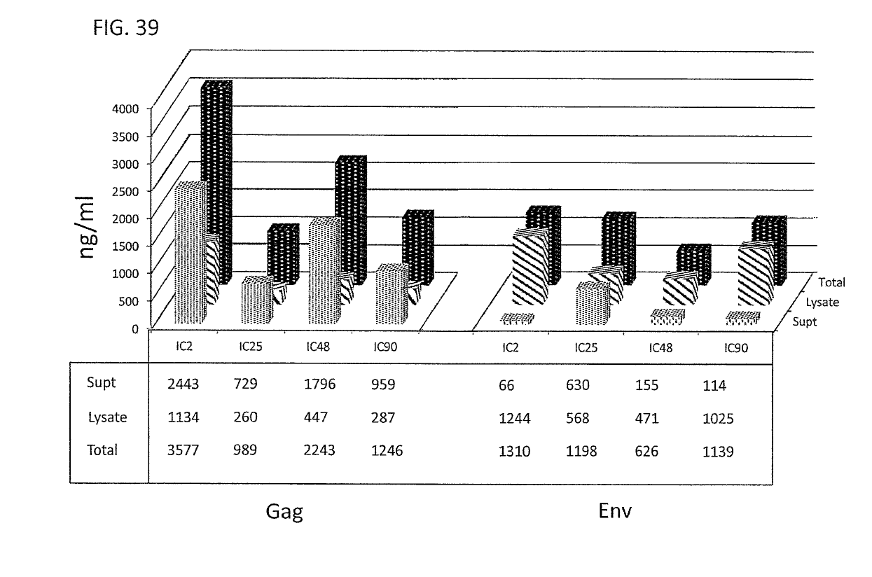

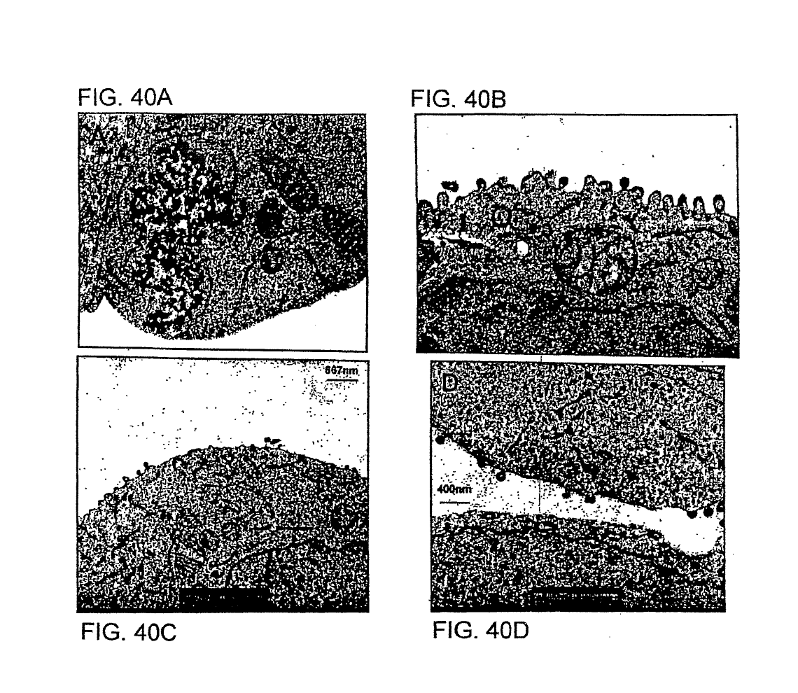

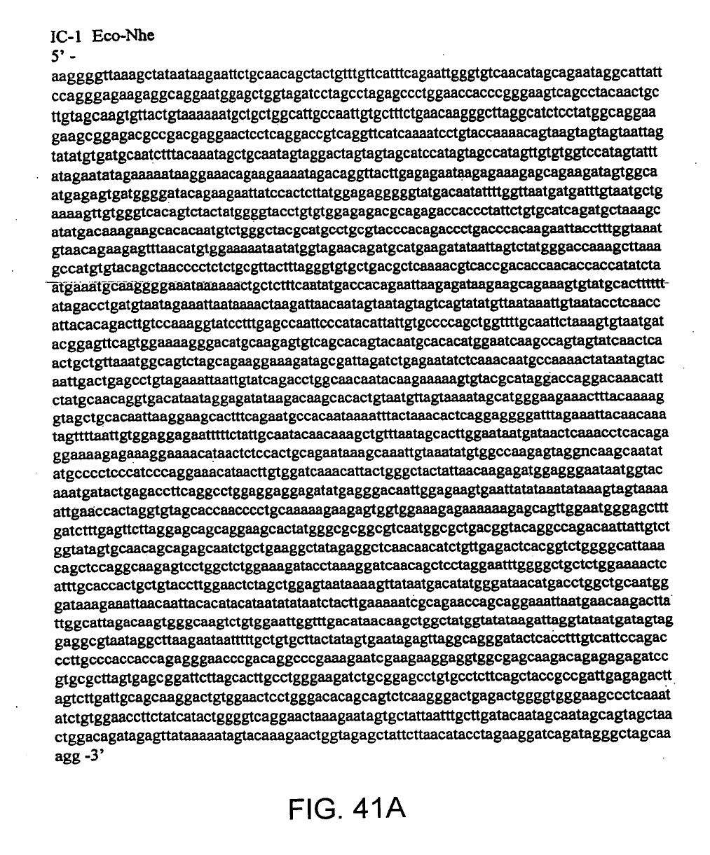

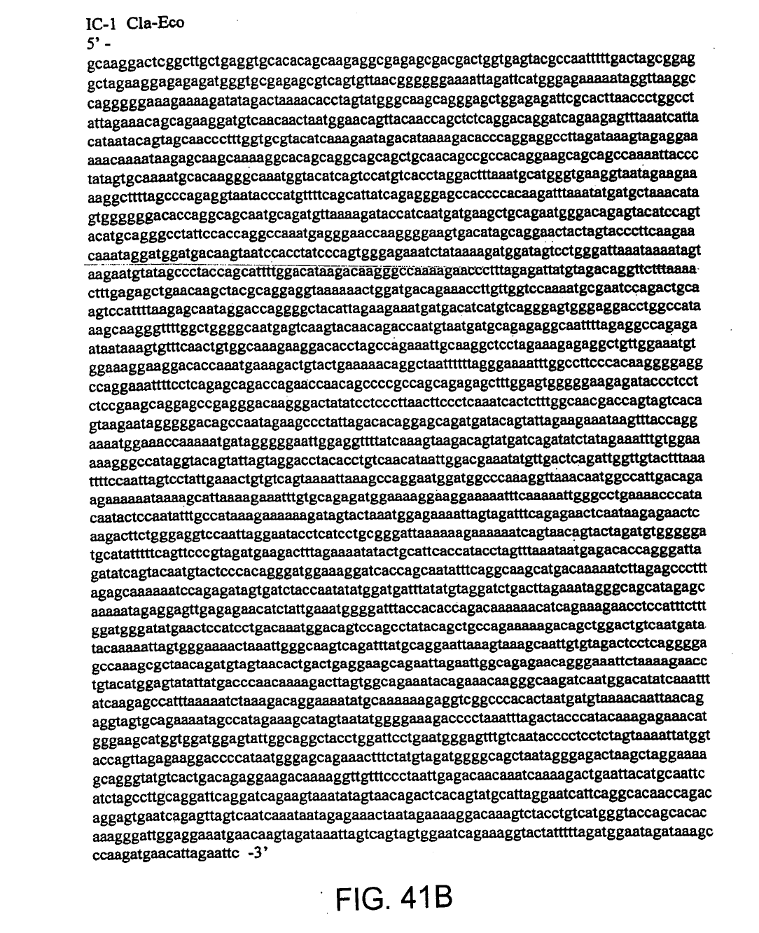

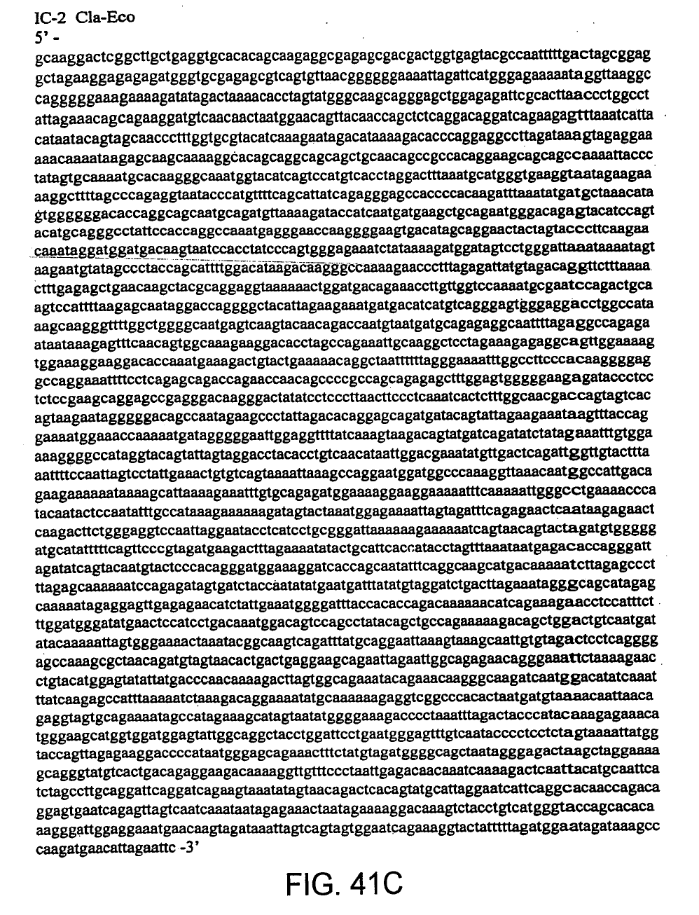

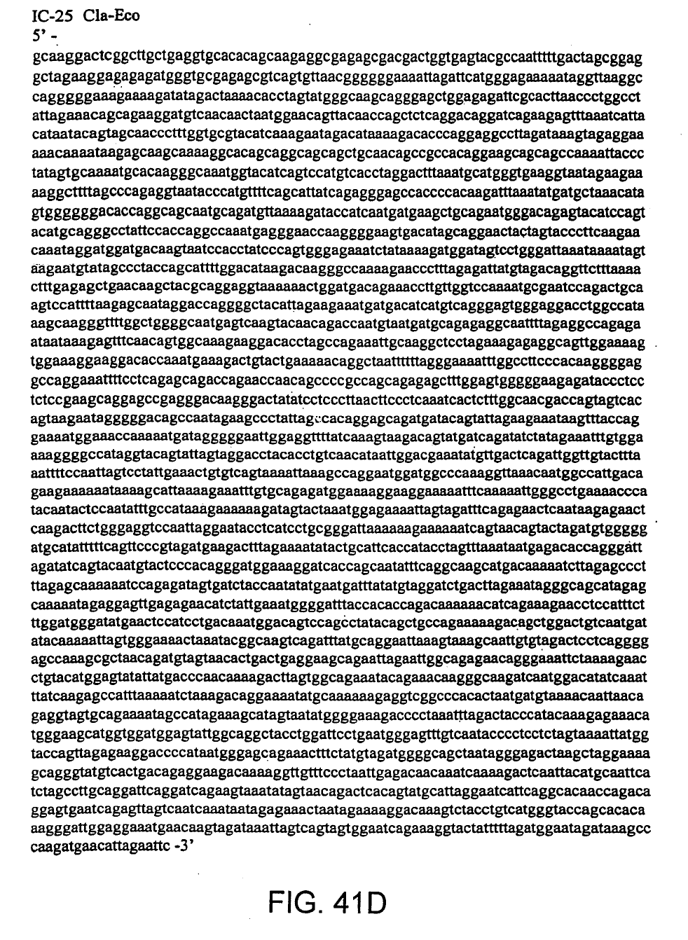

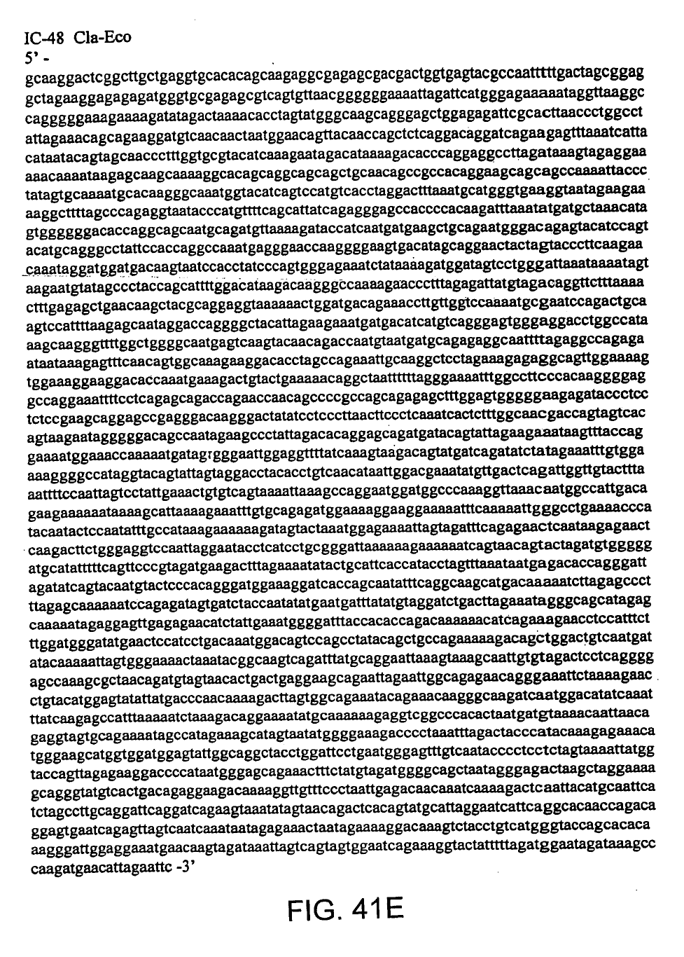

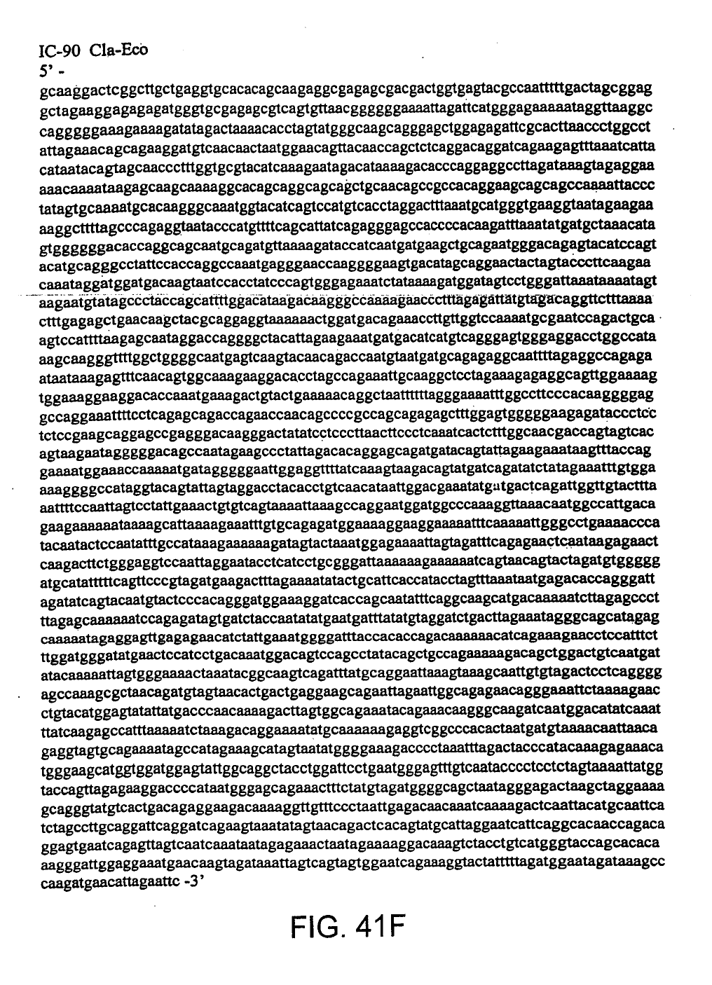

A patient isolate (#928, from the Ivory Coast) was isolated, characterized, and cloned at the Centers for Disease Control (Atlanta, Ga.). The clone was then used as the basis for several new clones, which can be used to generate vaccines, as described herein, against HIV clade AG. The first clone constructed is referred to herein as IC-1. The strategy used to construct IC-2 from IC-1 was the same as that used to construct pGA2/JS2 (a clade B isolate). The zinc finger and RT mutations are the same at the amino acid level. Additional clones were constructed with mutations in the viral protease gene. This was done to mimic the successful production of true VLPs observed with pGA2/JS7. Three different mutations were made in separate clones: D25A (IC-25), G48V (IC-48), and L90M (IC-90). A schematic representation of clade AG vaccine inserts (pGANC1/IC2, pGAV1/IC25, pGA1/IC48 and pGA1/IC90 are shown in FIG. 38. Each mutation differs in the overall effect of protease function. While characterization is still ongoing (see the expression data in FIG. 39), all mutations were successful in promoting particle formation (see the electron micrographs shown in FIGS. 40A-40D. The sequences of IC1 (Cla-Eco and Eco-Nhe), IC2, IC25, IC48, and IC90 are shown in FIGS. 41A-41F.

What is claimed is:

1. A pharmaceutically acceptable composition comprising a pox viral vector that encodes at least two antigens and, when administered to a patient, induces or enhances a first immune response directed against an antigen of a pathogen, provided the pathogen is not a pox virus, and a second immune response directed against an antigen that is obtained or derived from the pox viral vector.

2. The composition of claim 1, wherein the pox viral vector is a recombinant vaccinia Ankara (rMV A) virus.

3. The composition of claim 1, further comprising a second vector comprising the nucleotide sequence SEQ ID NO:1, SEQ ID NO:2, or SEQ ID NO:3 or variants thereof that retain substantially all of the biological activity of the vector.

4. The composition of claim 1, wherein the first antigen expressed by the poxviral vector is selected from the group consisting of HIV Gag, HIV gp120, HIV Pol, HIVEnv, HIV Tat, HIV Rev, HIV Vpu, HIV Nef, HIV Vif, HIV Vpr, HIV VLP, measles fusion protein, measles nucleoprotein, and a viral hemagglutinin, or biologically active mutants or fragments thereof.

5. The composition of claim 4, wherein the viral hemagglutinin is a measles virus hemagglutinin or an influenza viral hemagglutinin.

6. The composition of claim 1, further comprising a physiologically acceptable carrier, diluent, or excipient.

7. The composition of claim 1, further comprising a physiologically acceptable adjuvant.

8. The composition of claim 1, formulated for administration by a mucosal route, a parenteral route, or a transcutaneous route.

9. The composition of claim 1, wherein the first antigen expressed by the poxviral vector is further selected from the group consisting of HIV Gag, HIV gp120, HIVPol, HIV En, HIV Tat, HIV Rev, HIV Vpu, HIV Nef, HIV Vif, HIV Vpr, and HIV VLP, or mutants or fragments thereof.

10. The composition of claim 1, wherein the first antigen expressed by the poxviral vector is a polypeptide derived from an HIV VLP.

11. The composition of claim 1, wherein the first antigen expressed by the poxviral vector is derived from an Env-defective HIV VLP.

12. The vaccine of claim 1, wherein the second immune response is directed to an antigen of a variola virus.


SEQUENCE LISTING (52 sequences)

SEQ ID NO: 1 - DNA, 3894 bp, Artificial Sequence
  Vaccine Vector pGA1
cgacaatattggctattggccattgcatacgttgtatctatatcataatatgtacatttatattggctcatgtccaatatgaccgccatgttgacattgattattgactagttattaatagtaatcaattacgggttcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagactctataggcacacccctttggctcttatgcatgctatactgtttttggcttggggcctatacacccccgcttccttatgctataggtgatggtatagcttagcctataggtgtgggttattgaccattattgaccactcccctattggtgacgatactttccattactaatccataacatggctctttgccacaactatctctattggctatatgccaatactctgtccttcagagactgacacggactctgtatttttacaggatggggtcccatttattatttacaaattcacatatacaacaacgccgtcccccgtgcccgcagtttttattaaacatagcgtgggatctccacgcgaatctcgggtacctgttccggacatgggytcttctccggtagcggcggagcttccacatccgagccctggtcccatgcctccagcggctcatggtcgctcggcagctccttgctcctaacagtggaggccagacttaggcacagcacaatgcccaccaccaccagtgtgccgcacaaggccgtggcggtagggtatgtgtctgaaaatgagctcggagattgggctcgcaccgctgacgcagatggaagacttaaggcagcggcagaagaagatgcaggcagctgagttgttgtattctgataagagtcagaggtaactcccgttgcggtgctgttaacggtggagggcagtgtagtctgagcagtactcgttgctgccgcgcgcgccaccagacataatagctgacagactaacagactgttcctttccatgggtcttttctgcagtcaccatcgatgcttgcaatcatggatgcaatgaagagagggctctgctgtgtgctgctgctgtgtggagcagtcttcgtttcggctagccccgggtgataaacggaccgcgcaatccctaggctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatataaaaaacgcccggcggcaaccgagcgttctgaacgctagagtcgacaaattcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtctgccacacccagccggccacagtcgatgaatccagaaaagcggccattttccaccatgatattcggcaagcaggcatcgccatgggtcacgacgagatcctcgccgtcgggcatgctcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcttgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagatcttgatcccctgcgccatcagatccttggcggcaagaaagccatccagtttactttgcagggcttcccaaccttaccagagggcgccccagctggcaattccggttcgcttgctgtccataaaaccgcccagtctagctatcgccatgtaagcccactgcaagctacctgctttctctttgcgcttgcgttttcccttgtccagatagcccagtagctgacattcatccggggtcagcaccgtttctgcggactggctttctacgtgaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcccttttacggttcctggccttttgctggccttttgctcacatgttgt

SEQ ID NO: 2 - DNA, 2947 bp, Artificial Sequence
  Vaccine vector pGA2
cgacaatattggctattggccattgcatacgttgtatctatatcataatatgtacatttatattggctcatgtccaatatgaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaactcattctatcgatgcttgcaatcatggatgcaatgaagagagggctctgctgtgtgctgctgctgtgtggagcagtcttcgtttcggctagccccgggtgataaacggaccgcgcaatccctaggctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatataaaaaacgcccggcggcaaccgagcgttctgaacgctagagtcgacaaattcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtctgccacacccagccggccacagtcgatgaatccagaaaagcggccattttccaccatgatattcggcaagcaggcatcgccatgggtcacgacgagatcctcgccgtcgggcatgctcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcttgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagatcttgatcccctgcgccatcagatccttggcggcaagaaagccatccagtttactttgcagggcttcccaaccttaccagagggcgccccagctggcaattccggttcgcttgctgtccataaaaccgcccagtctagctatcgccatgtaagcccactgcaagctacctgctttctctttgcgcttgcgttttcccttgtccagatagcccagtagctgacattcatccggggtcagcaccgtttctgcggactggctttctacgtgaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcccttttacggttcctggccttttgctggccttttgctcacatgttgt

SEQ ID NO: 3 - DNA, 3893 bp, Artificial Sequence
  Vaccine Vector pGA3
cgacaatattggctattggccattgcatacgttgtatctatatcataatatgtacatttatattggctcatgtccaatatgaccgccatgttgacattgattattgactagttattaatagtaatcaattacggggtcattagttcatagcccatatatggagttccgcgttacataacttacggtaaatggcccgcctggctgaccccccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagactctataggcacacccctttggctcttatgcatgctatactgtttttggcttggggcctatacacccccgcttccttatgctataggtgatggtatagcttagcctataggtgtgggttattgaccattattgaccactcccctattggtgacgatactttccattactaatccataacatggctctttgccacaactatctctattggctatatgccaatactctgtccttcagagactgacacggactctgtatttttacaggatggggtcccatttattatttacaaattcacatatacaacaacgccgtcccccgtgcccgcagtttttattaaacatagcgtgggatctccacgcgaatctcgggtacgtgttccggacatgggctcttctccggtagcggcggagcttccacatccgagccctggtcccatgcctccagcggctcatggtcgctcggcagctccttgctcctaacagtggaggccagacttaggcacagcacaatgcccaccaccaccagtgtgccgcacaaggccgtggcggtagggtatgtgtctgaaaatgagctcggagattgggctcgcaccgctgacgcagatggaagacttaaggcagcggcagaagaagatgcaggcagctgagttgttgtattctgataagagtcagaggtaactcccgttgcggtgctgttaacggtggagggcagtgtagtctgagcagtactcgttgctgccgcgcgcgccaccagacataatagctgacagactaacagactgttcctttccatgggtcttttctgcagtcaccgtccaagcttgcaatcatggatgcaatgaagagagggctctgctgtgtgctgctgctgtgtggagcagtcttcgtttcggctagccccgggtgataaggatcctcgcaatccctaggctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatataaaaaacgcccggcggcaaccgagcgttctgaacgctagagtcgacaaattcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtctgccacacccagccggccacagtcgatgaatccagaaaagcggccattttccaccatgatattcggcaagcaggcatcgccatgggtcacgacgagatcctcgccgtcgggcatgctcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcttgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagatcttgatcccctgcgccatcagatccttggcggcaagaaagccatccagtttactttgcagggcttcccaaccttaccagagggcgccccagctggcaattccggttcgcttgctgtccataaaaccgcccagtctagctatcgccatgtaagcccactgcaagctacctgctttctctttgcgcttgcgttttcccttgtccagatagcccagtagctgacattcatccggggtcagcaccgtttctgcggactggctttctacgtgaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcccttttacggttcctggccttttgctggccttttgctcacatgttgt

SEQ ID NO: 4 - DNA, 9545 bp, Artificial Sequence
  Construct of vaccine vector pGA2 and insert JS2 expressing clade HIV-1 VL
atcgatgcaggactcggcttgctgaagcgcgcacggcaagaggcgaggggcggcgactggtgggtacgccaaaaattttgactagcggaggctagaaggagagagatgggtgcgagagcgtcagtattaagcgggggagaattagatcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaaaaaagcacagcaagcagcagctgacacaggacacagcagtcaggtcagccaaaattaccctatagtgcagaacatccaggggcaaatggtacatcaggccatatcacctagaactttaaatgcatgggtaaaagtagtagaagagaaggctttcagcccagaagtaatacccatgttttcagcattatcagaaggagccaccccacaagatttaaacaccatgctaaacacagtggggggacatcaagcagccatgcaaatgttaaaagagaccatcaatgaggaagctgcagaatgggatagagtacatccagtgcatgcagggcctattgcaccaggccagatgagagaaccaaggggaagtgacatagcaggaactactagtacccttcaggaacaaataggatggatgacaaataatccacctatcccagtaggagaaatttataaaagatggataatcctgggattaaataaaatagtaagaatgtatagccctaccagcattctggacataagacaaggaccaaaagaaccttttagagactatgtagaccggttctataaaactctaagagccgagcaagcttcacaggaggtaaaaaattggatgacagaaaccttgttggtccaaaatgcgaacccagattgtaagactattttaaaagcattgggaccagcggctacactagaagaaatgatgacagcatgtcagggagtaggaggacccggccataaggcaagagttttggctgaagcaatgagccaagtaacaaatacagctaccataatgatgcagagaggcaattttaggaaccaaagaaagatggttaagagcttcaatagcggcaaagaagggcacacagccagaaattgcagggcccctaggaaaaagggcagctggaaaagcggaaaggaaggacaccaaatgaaagattgtactgagagacaggctaattttttagggaagatctggccttcctacaagggaaggccagggaattttcttcagagcagaccagagccaacagccccaccatttcttcagagcagaccagagccaacagccccaccagaagagagcttcaggtctggggtagagacaacaactccccctcagaagcaggagccgatagacaaggaactgtatcctttaacttccctcagatcactctttggcaacgacccctcgtcacaataaagataggggggcaactaaaggaagctctattagatacaggagcagatgatacagtattagaagaaatgagtttgccaggaagatggaaaccaaaaatgatagggggaattggaggttttatcaaagtaagacagtatgatcagatactcatagaaatctgtggacataaagctataggtacagtattagtaggacctacacctgtcaacataattggaagaaatctgttgactcagattggttgcactttaaattttcccattagccctattgagactgtaccagtaaaattaaagccaggaatggatggcccaaaagttaaacaatggccattgacagaagaaaaaataaaagcattagtagaaatttgtacagaaatggaaaaggaagggaaaatttcaaaaattgggcctgagaatccatacaatactccagtatttgccataaagaaaaaagacagtactaaatggagaaaattagtagatttcagagaacttaataagagaactcaagacttctgggaagttcaattaggaataccacatcccgcagggttaaaaaagaaaaaatcagtaacagtactggatgtgggtgatgcatatttttcagttcccttagatgaagacttcaggaagtatactgcatttaccatacctagtataaacaatgagacaccagggattagatatcagtacaatgtgcttccacagggatggaaaggatcaccagcaatattccaaagtagcatgacaaaaatcttagagccttttaaaaaacaaaatccagacatagttatctatcaatacatgaacgatttgtatgtaggatctgacttagaaatagggcagcatagaacaaaaatagaggagctgagacaacatctgttgaggtggggacttaccacaccagacaaaaaacatcagaaagaacctccattcctttggatgggttatgaactccatcctgataaatggacagtacagcctatagtgctgccagaaaaagacagctggactgtcaatgacatacagaagttagtggggaaattgaataccgcaagtcagatttacccagggattaaagtaaggcaattatgtaaactccttagaggaaccaaagcactaacagaagtaataccactaacagaagaagcagagctagaactggcagaaaacagagagattctaaaagaaccagtacatggagtgtattatgacccatcaaaagacttaatagcagaaatacagaagcaggggcaaggccaatggacatatcaaatttatcaagagccatttaaaaatctgaaaacaggaaaatatgcaagaatgaggggtgcccacactaatgatgtaaaacaattaacagaggcagtgcaaaaaataaccacagaaagcatagtaatatggggaaagactcctaaatttaaactacccatacaaaaggaaacatgggaaacatggtggacagagtattggcaagccacctggattcctgagtgggagtttgttaatacccctcctttagtgaaattatggtaccagttagagaaagaacccatagtaggagcagaaaccttctatgtagatggggcagctaacagggagactaaattaggaaaagcaggatatgttactaacaaaggaagacaaaaggttgtccccctaactaacacaacaaatcagaaaactcagttacaagcaatttatctagctttgcaggattcaggattagaagtaaacatagtaacagactcacaatatgcattaggaatcattcaagcacaaccagataaaagtgaatcagagttagtcaatcaaataatagagcagttaataaaaaaggaaaaggtctatctggcatgggtaccagcacacaaaggaattggaggaaatgaacaagtagataaattagtcagtgctggaatcaggaaaatactatttttagatggaatagataaggcccaagatgaacattagaattctgcaacaactgctgtttatccattttcagaattgggtgtcgacatagcagaataggcgttactcgacagaggagagcaagaaatggagccagtagatcctagactagagccctggaagcatccaggaagtcagcctaaaactgcttgtaccaattgctattgtaaaaagtgttgctttcattgccaagtttgtttcataacaaaagccttaggcatctcctatggcaggaagaagcggagacagcgacgaagacctcctcaagacagtcagactcatcaagtttctctatcaaagcagtaagtagtaaatgtaatgcaacctttacaaatattagcaatagtagcattagtagtagcagcaataatagcaatagttgtgtggaccatagtattcatagaatataggaaaatattaagacaaagaaaaatagacaggttaattgataggataacagaaagagcagaagacagtggcaatgaaagtgaaggggatcaggaagaattatcagcacttgtggaaatggggcatcatgctccttgggatgttgatgatctgtagtgctgtagaaaatttgtgggtcacagtttattatggggtacctgtgtggaaagaagcaaccaccactctattttgtgcatcagatgctaaagcatatgatacagaggtacataatgtttgggccacacatgcctgtgtacccacagaccccaacccacaagaagtagtattggaaaatgtgacagaaaattttaacatgtggaaaaataacatggtagaacagatgcatgaggatataatcagtttatgggatcaaagcctaaagccatgtgtaaaattaaccccactctgtgttactttaaattgcactgatttgaggaatgttactaatatcaataatagtagtgagggaatgagaggagaaataaaaaactgctctttcaatatcaccacaagcataagagataaggtgaagaaagactatgcacttttttatagacttgatgtagtaccaatagataatgataatactagctataggttgataaattgtaatacctcaaccattacacaggcctgtccaaaggtatcctttgagccaattcccatacattattgtaccccggctggttttgcgattctaaagtgtaaagacaagaagttcaatggaacagggccatgtaaaaatgtcagcacagtacaatgtacacatggaattaggccagtagtgtcaactcaactgctgttaaatggcagtctagcagaagaagaggtagtaattagatctagtaatttcacagacaatgcaaaaaacataatagtacagttgaaagaatctgtagaaattaattgtacaagacccaacaacaatacaaggaaaagtatacatataggaccaggaagagcattttatacaacaggagaaataataggagatataagacaagcacattgcaacattagtagaacaaaatggaataacactttaaatcaaatagctacaaaattaaaagaacaatttgggaataataaaacaatagtctttaatcaatcctcaggaggggacccagaaattgtaatgcacagttttaattgtggaggggaatttttctactgtaattcaacacaactgtttaatagtacttggaattttaatggtacttggaatttaacacaatcgaatggtactgaaggaaatgacactatcacactcccatgtagaataaaacaaattataaatatgtggcaggaagtaggaaaagcaatgtatgcccctcccatcagaggacaaattagatgctcatcaaatattacagggctaatattaacaagagatggtggaactaacagtagtgggtccgagatcttcagacctgggggaggagatatgagggacaattggagaagtgaattatataaatataaagtagtaaaaattgaaccattaggagtagcacccaccaaggcaaaaagaagagtggtgcagagagaaaaaagagcagtgggaacgataggagctatgttccttgggttcttgggagcagcaggaagcactatgggcgcagcgtcaataacgctgacggtacaggccagactattattgtctggtatagtgcaacagcagaacaatttgctgagggctattgaggcgcaacagcatctgttgcaactcacagtctggggcatcaagcagctccaggcaagagtcctggctctggaaagatacctaagggatcaacagctcctagggatttggggttgctctggaaaactcatctgcaccactgctgtgccttggaatgctagttggagtaataaaactctggatatgatttgggataacatgacctggatggagtgggaaagagaaatcgaaaattacacaggcttaatatacaccttaattgaagaatcgcagaaccaacaagaaaagaatgaacaagacttattagcattagataagtgggcaagtttgtggaattggtttgacatatcaaattggctgtggtgtataaaaatcttcataatgatagtaggaggcttgataggtttaagaatagtttttactgtactttctatagtaaatagagttaggcagggatactcaccattgtcatttcagacccacctcccagccccgaggggacccgacaggcccgaaggaatcgaagaagaaggtggagacagagacagagacagatccgtgcgattagtggatggatccttagcacttatctgggacgatctgcggagcctgtgcctcttcagctaccaccgcttgagagacttactcttgattgtaacgaggattgtggaacttctgggacgcagggggtgggaagccctcaaatattggtggaatctcctacagtattggagtcaggagctaaagaatagtgctgttagcttgctcaatgccacagctatagcagtagctgaggggacagatagggttatagaagtagtacaaggagcttatagagctattcgccacatacctagaagaataagacagggcttggaaaggattttgctataagatgggtggctagccccgggtgataaacggaccgcgcaatccctaggctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatataaaaaacgcccggcggcaaccgagcgttctgaacgctagagtcgacaaattcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtctgccacacccagccggccacagtcgatgaatccagaaaagcggccattttccaccatgatattcggcaagcaggcatcgccatgggtcacgacgagatcctcgccgtcgggcatgctcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcttgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagatcttgatcccctgcgccatcagatccttggcggcgagaaagccatccagtttactttgcagggcttcccaaccttaccagagggcgccccagctggcaattccggttcgcttgctgtccataaaaccgcccagtctagctatcgccatgtaagcccactgcaagctacctgctttctctttgcgcttgcgttttcccttgtccagatagcccagtagctgacattcatccggggtcagcaccgtttctgcggactggctttctacgtgaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctgggcttttgctggccttttgctcacatgttgtcgaccgacaatattggctattggccattgcatacgttgtatctatatcataatatgtacatttatattggctcatgtccaatatgaccgccatgttgacattgattattgactagttattaatagtaatcaattacggggtcattagttcatagcccatatatggagttccgcgttacataacttacggtaaatggcccgcctcgtgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgcccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatcgc

SEQ ID NO: 5 - DNA, 9918 bp, Artificial Sequence
  Construct of vaccine vector pGA1 and vaccine insert expressing clade B HIV-1 Gag-Po
atcgatgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaaaaaagcacagcaagcagcagctgacacaggacacagcagtcaggtcagccaaaattaccctatagtgcagaacatccaggggcaaatggtacatcaggccatatcacctagaactttaaatgcatgggtaaaagtagtagaagagaaggctttcagcccagaagtaatacccatgttttcagcattatcagaaggagccaccccacaagatttaaacaccatgctaaacacagtggggggacatcaagcagccatgcaaatgttaaaagagaccatcaatgaggaagctgcagaatgggatagagtacatccagtgcatgcagggcctattgcaccaggccagatgagagaaccaaggggaagtgacatagcaggaactactagtacccttcaggaacaaataggatggatgacaaataatccacctatcccagtaggagaaatttataaaagatggataatcctgggattaaataaaatagtaagaatgtatagccctaccagcattctggacataagacaaggaccaaaagaaccttttagagactatgtagaccggttctataaaactctaagagccgagcaagcttcacaggaggtaaaaaattggatgacagaaaccttgttggtccaaaatgcgaacccagattgtaagactattttaaaagcattgggaccagcggctacactagaagaaatgatgacagcatgtcagggagtaggaggacccggccataaggcaagagttttggctgaagcaatgagccaagtaacaaatacagctaccataatgatgcagagaggcaattttaggaaccaaagaaagatggttaagagcttcaatagcggcaaagaagggcacacagccagaaattgcagggcccctaggaaaaagggcagctggaaaagcggaaaggaaggacaccaaatgaaagattgtactgagagacaggctaattttttagggaagatctggccttcctacaagggaaggccagggaattttcttcagagcagaccagagccaacagccccaccatttcttcagagcagaccagagccaacagccccaccagaagagagcttcaggtctggggtagagacaacaactccccctcagaagcaggagccgatagacaaggaactgtatcctttaacttccctcagatcactctttggcaacgacccctcgtcacaataaagataggggggcaactaaaggaagctctattagatacaggagcagatgatacagtattagaagaaatgagtttgccaggaagatggaaaccaaaaatgatagggggaattggaggttttatcaaagtaagacagtatgatcagatactcatagaaatctgtggacataaagctataggtacagtattagtaggacctacacctgtcaacataattggaagaaatctgttgactcagattggttgcactttaaattttcccattagccctattgagactgtaccagtaaaattaaagccaggaatggatggcccaaaagttaaacaatggccattgacagaagaaaaaataaaagcattagtagaaatttgtacagaaatggaaaaggaagggaaaatttcaaaaattgggcctgagaatccatacaatactccagtatttgccataaagaaaaaagacagtactaaatggagaaaattagtagatttcagagaacttaataagagaactcaagacttctgggaagttcaattaggaataccacatcccgcagggttaaaaaagaaaaaatcagtaacagtactggatgtgggtgatgcatatttttcagttcccttagatgaagacttcaggaagtatactgcatttaccatacctagtataaacaatgagacaccagggattagatatcagtacaatgtgcttccacagggatggaaaggatcaccagcaatattccaaagtagcatgacaaaaatcttagagccttttaaaaaacaaaatccagacatagttatctatcaatacatgaacgatttgtatgtaggatctgacttagaaatagggcagcatagaacaaaaatagaggagctgagacaacatctgttgaggtggggacttaccacaccagacaaaaaacatcagaaagaacctccattcctttggatgggttatgaactccatcctgataaatggacagtacagcctatagtgctgccagaaaaagacagctggactgtcaatgacatacagaagttagtggggaaattgaataccgcaagtcagatttacccagggattaaagtaaggcaattatgtaaactccttagaggaaccaaagcactaacagaagtaataccactaacagaagaagcagagctagaactggcagaaaacagagagattctaaaagaaccagtacatggagtgtattatgacccatcaaaagacttaatagcagaaatacagaagcaggggcaaggccaatggacatatcaaatttatcaagagccatttaaaaatctgaaaacaggaaaatatgcaagaatgaggggtgcccacactaatgatgtaaaacaattaacagaggcagtgcaaaaaataaccacagaaagcatagtaatatggggaaagactcctaaatttaaactacccatacaaaaggaaacatgggaaacatggtggacagagtattggcaagccacctggattcctgagtgggagtttgttaatacccctcctttagtgaaattatggtaccagttagagaaagaacccatagtaggagcagaaaccttctatgtagatggggcagctaacagggagactaaattaggaaaagcaggatatgttactaacaaaggaagacaaaaggttgtccccctaactaacacaacaaatcagaaaactcagttacaagcaatttatctagctttgcaggattcaggattagaagtaaacatagtaacagactcacaatatgcattaggaatcattcaagcacaaccagataaaagtgaatcagagttagtcaatcaaataatagagcagttaataaaaaaggaaaaggtctatctggcatgggtaccagcacacaaaggaattggaggaaatgaacaagtagataaattagtcagtgctggaatcaggaaaatactatttttagatggaatagataaggcccaagatgaacattagaattctgcaacaactgctgtttatccattttcagaattgggtgtcgacatagcagaataggcgttactcgacagaggagagcaagaaatggagccagtagatcctagactagagccctggaagcatccaggaagtcagcctaaaactgcttgtaccaattgctattgtaaaaagtgttgctttcattgccaagtttgtttcataacaaaagccttaggcatctcctatggcaggaagaagcggagacagcgacgaagacctcctcaaggcagtcagactcatcaagtttctctatcaaagcagtaagtagtacatgtaatgcaacctatacaaatagcaatagtagcattagtagtagcaataataatagcaatagttgtgtggtccatagtaatcatagaatataggaaaatattaagacaaagaaaaatagacaggttaattgatagactaatagaaagagcagaagacagtggcaatgagagtgaaggagaaatatcagcacttgtggagatgggggtggagatggggcaccatgctccttgggatgttgatgatctgtagtgctacagaaaaattgtgggtcacagtctattatggggtacctgtgtggaaggaagcaaccaccactctattttgtgcatcagatgctaaagcatatgatacagaggtacataatgtttgggccacacatgcctgtgtacccacagaccccaacccacaagaagtagtattggtaaatgtgacagaaaattttaacatgtggaaaaatgacatggtagaacagatgcatgaggatataatcagtttatgggatcaaagcctaaagccatgtgtaaaattaaccccactctgtgttagtttaaagtgcactgatttgaagaatgatactaataccaatagtagtagcgggagaatgataatggagaaaggagagataaaaaactgctctttcaatatcagcacaagcataagaggtaaggtgcagaaagaatatgcatttttttataaacttgatataataccaatagataatgatactaccagctatacgttgacaagttgtaacacctcagtcattacacaggcctgtccaaaggtatcctttgagccaattcccatacattattgtgccccggctggttttgcgattctaaaatgtaataataagacgttcaatggaacaggaccatgtacaaatgtcagcacagtacaatgtacacatggaattaggccagtagtatcaactcaactgctgttaaatggcagtctggcagaagaagaggtagtaattagatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaataacatgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctgtagtgaatagagttaggcagggatattcaccattatcgtttcagacccacctcccaatcccgaggggacccgacaggcccgaaggaatagaagaagaaggtggagagagagacagagacagatccattcgattagtgaacggatccttagcacttatctgggacgatctgcggagcctgtgcctcttcagctaccaccgcttgagagacttactcttgattgtaacgaggattgtggaacttctgggacgcagggggtgggaagccctcaaatattggtggaatctcctacagtattggagtcaggagctaaagaatagtgctgttagcttgctcaatgccacagctatagcagtagctgaggggacagatagggttatagaagtagtacaaggagcttatagagctattcgccacatacctagaagaataagacagggcttggaaaggattttgctataagatgggtggctagccccgggtgataaacggaccgcgcaatccctaggctgtgccttctagttgccagccaaactgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatataaaaaacgcccggcggcaaccgagcgttctgaacgctagagtcgacaaattcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgctcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcttgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagatcttgatcccctgcgccatcagatccttggcggcgagaaagccatccagtttactttgcagggcttcccaaccttaccagagggcgccccagctggcaattccggttcgcttgctgtccataaaaccgcccagtctagctatcgccatgtaagcccactgcaagctacctgctttctctttgcgcttgcgttttcccttgtccagatagcccagtagctgacattcatccggggtcagcaccgtttctgcggactggctttctacgtgaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctgggcttttgctggccttttgctcacatgttgtcgaccgacaatattggctattggccattgcatacgttgtatctatatcataatatgtacatttatattggctcatgtccaatatgaccgccatgttgacattgattattgactagttattaatagtaatcaattacggggtcattagttcatagcccatatatggagttccgcgttacataacttacggtaaatggcccgcctcgtgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgcccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagactctataggcacacccctttggctcttatgcatgctatactgtttttggcttggggcctatacacccccgctccttatgctataggtgatggtatagcttagcctataggtgtgggttattgaccattattgaccactcccctattggtgacgatactttccattactaatccataacatggctctttgccacaactatctctattggctatatgccaatactctgtccttcagagactgacacggactctgtatttttacaggatggggtcccatttattatttacaaattcacatatacaacaacgccgtcccccgtgcccgcagtttttattaaacatagcgtgggatctccacgcgaatctcgggtacgtgttccggacatgggctcttctccggtagcggcggagcttccacatccgagccctggtcccatgcctccagcggctcatggtcgctcggcagctccttgctcctaacagtggaggccagacttaggcacagcacaatgcccaccaccaccagtgtgccgcacaaggccgtggcggtagggtatgtgtctgaaaatgagctcggagattgggctcgcaccgtgacgcagatggaagacttaaggcagcggcagaagaagatgcaggcagctgagttgttgtattctgataagagtcagaggtaactcccgttgcggtgctgttaacggtggagggcagtgtagtctgagcagtactcgttgctgccgcgcgcgccaccagacataatagctgacagactaacagactgttcctttccatgggtcttttctgcagtcaccgtcca

SEQ ID NO: 6 - DNA, 35 bp, Artificial Sequence
  introduced lambda T0 terminator sequence
ataaaaaacgcccggcggcaaccgagcgttctgaa

SEQ ID NO: 7 - DNA, 30 bp, Artificial Sequence
  primer for site-directed mutagenesis for introducing Cla I site
ccgtcagatcgcatcgatacgccatccacg

SEQ ID NO: 8 - DNA, 30 bp, Artificial Sequence
  primer for site-directed mutagenesis to introduce Cla I site
cgtggatggcgtatcgatgcgatctgacgg

SEQ ID NO: 9 - DNA, 29 bp, Artificial Sequence
  Sense primer
gagctctatcgatgcaggactcggcttgc

SEQ ID NO: 10 - DNA, 31 bp, Artificial Sequence
  antisense primer
ggcaggttttaatcgctagcctatgctctcc

SEQ ID NO: 11 - DNA, 17 bp, Artificial Sequence
  Sense Primer
gggcaggagtgctagcc

SEQ ID NO: 12 - DNA, 29 bp, Artificial Sequence
  Antisense Primer
ccacactactttcggaccgctagccaccc

SEQ ID NO: 13 - DNA, 32 bp, Artificial Sequence
  C15S ZN1 primer
ggttaagagcttcaatagcggcaaagaagggc

SEQ ID NO: 14 - DNA, 32 bp, Artificial Sequence
  C15S ZN2 primer
gcccttctttgccgctattgaagctcttaacc

SEQ ID NO: 15 - DNA, 27 bp, Artificial Sequence
  C36S ZN3 primer
gggcagctggaaaagcggaaaggaagg

SEQ ID NO: 16 - DNA, 27 bp, Artificial Sequence
  C36S ZN4 primer
ccttcctttccgcttttccagctgccc

SEQ ID NO: 17 - DNA, 44 bp, Artificial Sequence
  D185N RT1 primer
ccagacatagttatctatcaatacatgaacgatttgtatgtagg

SEQ ID NO: 18 - DNA, 44 bp, Artificial Sequence
  D185N RT2 primer
cctacatacaaatcgttcatgtattgatagataactatgtctgg

SEQ ID NO: 19 - DNA, 33 bp, Artificial Sequence
  W266T RT3 primer
ggggaaattgaataccgcaagtcagatttaccc

SEQ ID NO: 20 - DNA, 33 bp, Artificial Sequence
  W266T RT4 primer
gggtaaatctgacttgcggtattcaatttcccc

SEQ ID NO: 21 - DNA, 40 bp, Artificial Sequence
  E478Q RT5 primer
ccctaactaacacaacaaatcagaaaactcagttacaagc

SEQ ID NO: 22 - DNA, 40 bp, Artificial Sequence
  E478Q RT6 primer
gcttgtaactgagttttctgatttgttgtgttagttaggg

SEQ ID NO: 23 - DNA, 34 bp, Artificial Sequence
  D25A Prt1 primer
ggcaactaaaggaagctctattagccacaggagc

SEQ ID NO: 24 - DNA, 34 bp, Artificial Sequence
  D25 Aprt2 primer
gctcctgtggctaatagagcttcctttagttgcc

SEQ ID NO: 25 - protein, 512 aa, Artificial Sequence
  Synthetically generated peptide
MGARASVLSGGELDRWEKIRLRPGGKKKYKLKHIVWASRELERFAVNPGLLETSEGCRQILGQLQPSLQTGSEELRSLYNTVATLYCVHQRIEIKDTKEALDKIEEEQNKSKKKAQQAAADTGHSSQVSQNYPIVQNIQGQMVHQAISPRTLNAWVKVVEEKAFSPEVIPMFSALSEGATPQDLNTMLNTVGGHQAAMQMLKETINEEAAEWDRVHPVHAGPIAPGQMREPRGSDIAGTTSTLQEQIGWMTNNPPIPVGEIYKRWIILGLNKIVRMYSPTSILDIRQGPKEPFRDYVDRFYKTLRAEQASQEVKNWMTETLLVQNANPDCKTILKALGPAATLEEMMTACQGVGGPGHKARVLAEAMSQVTNTATIMMQRGNFRNQRKMVKSFNSGKEGHTARNCRAPRKKGSWKSGKEGHQMKDCTERQANFLGKIWPSYKGRPGNFLQSRPEPTAPPFLQSRPEPTAPPEESFRSGVETTTPPQKQEPIDKELYPLTSLRSLFGNDPSSQ

SEQ ID NO: 26 - protein, 739 aa, Artificial Sequence
  Synthetically generated peptide
FFREDLAFLQGKAREFSSEQTRANSPTISSEQTRANSPTRRELQVWGRDNNSPSEAGADRQGTVSFNFPQITLWQRPLVTIKIGGQLKEALLDTGADDTVLEEMSLPGRWKPKMIGGIGGFIKVRQYDQILIEICGHKAIGTVLVGPTPVNIIGRNLLTQIGCTLNFPISPIETVPVKLKPGMDGPKVKQWPLTEEKIKALVEICTEMEKEGKISKIGPENPYNTPVFAIKKKDSTKWRKLVDFRELNKRTQDFWEVQLGIPHPAGLKKKKSVTVLDVGDAYFSVPLDEDFRKYTAFTIPSINNETPGIRYQYNVLPQGWKGSPAIFQSSMTKILEPFKKQNPDIVIYQYMNDLYVGSDLEIGQHRTKIEELRQHLLRWGLTTPDKKHQKEPPFLWMGYELHPDKWTVQPIVLPEKDSWTVNDIQKLVGKLNTASQIYPGIKVRQLCKLLRGTKALTEVIPLTEEAELELAENREILKEPVHGVYYDPSKDLIAEIQKQGQGQWTYQIYQEPFKNLKTGKYARMRGAHTNDVKQLTEAVQKITTESIVIWGKTPKFKLPIQKETWETWWTEYWQATWIPEWEFVNTPPLVKLWYQLEKEPIVGAETFYVDGAANRETKLGKAGYVTNKGRQKVVPLTNTTNQKTQLQAIYLALQDSGLEVNIVTDSQYALGIIQAQPDKSESELVNQIIEQLIKKEKVYLAWVPAHKGIGGNEQVDKLVSAGIRKILFLDGIDKAQDEH

SEQ ID NO: 27 - protein, 72 aa, Artificial Sequence
  Synthetically generated peptide
MEPVDPRLEPWKHPGSQPKTACTNCYCKKCCFHCQVCFITKALGISYGRKKRRQRRRPPQDSQTHQVSLSKQ

SEQ ID NO: 28 - protein, 25 aa, Artificial Sequence
  Synthetically generated peptide
MAGRSGDSDEDLLKTVRLIKFLYQS

SEQ ID NO: 29 - protein, 853 aa, Artificial Sequence
  Synthetically generated peptide
MKVKGIRKNYQHLWKWGIMLLGMLMICSAVENLWVTVYYGVPVWKEATTTLFCASDAKAYDTEVHNVWATHACVPTDPNPQEVVLENVTENFNMWKNNMVEQMHEDIISLWDQSLKPCVKLTPLCVTLNCTDLRNVTNINNSSEGMRGEIKNCSFNITTSIRDKVKKDYALFYRLDVVPIDNDNTSYRLINCNTSTITQACPKVSFEPIPIHYCTPAGFAILKCKDKKFNGTGPCKNVSTVQCTHGIRPVVSTQLLLNGSLAEEEVVIRSSNFTDNAKNIIVQLKESVEINCTRPNNNTRKSIHIGPGRAFYTTGEIIGDIRQAHCNISRTKWNNTLNQIATKLKEQFGNNKTIVFNQSSGGDPEIVMHSFNCGGEFFYCNSTQLFNSTWNFNGTWNLTQSNGTEGNDTITLPCRIKQIINMWQEVGKAMYAPPIRGQIRCSSNITGLILTRDGGTNSSGSEIFRPGGGDMRDNWRSELYKYKVVKIEPLGVAPTKAKRRVVQREKRAVGTIGAMFLGFLGAAGSTMGAASITLTVQARLLLSGIVQQQNNLLRAIEAQQHLLQLTVWGIKQLQARVLALERYLRDQQLLGIWGCSGKLICTTAVPWNASWSNKTLDMIWDNMTWMEWEREIENYTGLIYTLIEESQNQQEKNEQDLLALDKWASLWNWFDISNWLWCIKIFIMIVGGLIGLRIVFTVLSIVNRVRQGYSPLSFQTHLPAPRGPDRPEGIEEEGGDRDRDRSVRLVDGSLALIWDDLRSLCLFSYHRLRDLLLIVTRIVELLGRRGWEALKYWWNLLQYWSQELKNSAVSLLNATAIAVAEGTDRVIEVVQGAYRAIRHIPRRIRQGLERILL

SEQ ID NO: 30 - protein, 512 aa, Artificial Sequence; identical to SEQ ID NO: 25
  Synthetically generated peptide

SEQ ID NO: 31 - protein, 739 aa, Artificial Sequence; identical to SEQ ID NO: 26
  Synthetically generated peptide

SEQ ID NO: 32 - protein, 72 aa, Artificial Sequence
  Synthetically generated peptide
MEPVDPRLEPWKHPGSQPKTACTNCYCKKCCFHCQVCFITKALGISYGRKKRRQRRRPPQGSQTHQVSLSKQ

SEQ ID NO: 33 - protein, 25 aa, Artificial Sequence
  Synthetically generated peptide
MAGRSGDSDEDLLKAVRLIKFLYQS

SEQ ID NO: 34 - protein, 81 aa, Homo sapiens
MQPIQIAIVALVVAIIIAIVVWSIVIIEYRKILRQRKIDRLIDRLIERAEDSGNESEGEISALVEMGVEMGHHAPWDVDDL

SEQ ID NO: 35 - protein, 281 aa, Artificial Sequence
  Synthetically generated peptide
MRVKEKYQHLWRWGWRWGTMLLGMLMICSATEKLWVTVYYGVPVWKEATTTLFCASDAKAYDTEVHNVWATHACVPTDPNPQEVVLVNVTENFNMWKNDMVEQMHEDIISLWDQSLKPCVKLTPLCVSLKCTDLKNDTNTNSSSGRMIMEKGEIKNCSFNISTSIRGKVQKEYAFFYKLDIIPIDNDTTSYTLTSCNTSVITQACPKVSFEPIPIHYCAPAGFAILKCNNKTFNGTGPCTNVSTVQCTHGIRPVVSTQLLLNGSLAEEEVVIRSSDLEEEI

SEQ ID NO: 36 - protein, 21 aa, Artificial Sequence
  Synthetically generated peptide
MDAMKRGLCCVLLLCGAVFVS

SEQ ID NO: 37 - DNA, 3894 bp, Artificial Sequence
  Vaccine vector pGA1
acaacatgtgagcaaaaggccagcaaaaggccaggaaccgtaaaagggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttcacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcagaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaatttgtcgactctagcgttcagaacgctcggttgccgccgggcgttttttatatagagcccaccgcatccccagcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagcctagggattgcgcggtccgtttatcacccggggctagccgaaacgaagactgctccacacagcagcagcacacagcagagccctctcttcattgcatccatgattgcaagcatcgatggtgactgcagaaaagacccatggaaaggaacagtctgttagtctgtcagctattatgtctggtggcgcgcgcggcagcaacgagtactgctcagactacactgccctccaccgttaacagcaccgcaacgggagttacctctgactcttatcagaatacaacaactcagctgcctgcatcttcttctgccgctgccttaagtcttccatctgcgtcagcggtgcgagcccaatctccgagctcattttcagacacataccctaccgccacggccttgtgcggcacactggtggtggtgggcattgtgctgtgcctaagtctggcctccactgttaggagcaaggagctgccgagcgaccatgagccgctggaggcatgggaccagggctcggatgtggaagctccgccgctaccggagaagaycccatgtccggaacaggtacccgagattcgcgtggagatcccacgctatgtttaataaaaactgcgggcacgggggacggcgttgttgtatatgtgaatttgtaaataataaatgggaccccatcctgtaaaaatacagagtccgtgtcagtctctgaaggacagagtattggcatatagccaatagagatagttgtggcaaagagccatgttatggattagtaatggaaagtatcgtcaccaataggggagtggtcaataatggtcaataacccacacctataggctaagctataccatcacctatagcataaggaagcgggggtgtataggccccaagccaaaaacagtatagcatgcataagagccaaaggggtgtgcctatagagtctataggcggtacttacgtcactcttggcacggggaatccgcgttccaatgcaccgttcccggccgcggaggctggatcggtcccggtgtcttctatggaggtcaaaacagcgtggatggcgtctccaggcgatctgacggttcactaaacgagctctgcttatatagacctcccaccgtacacgcctaccgcccatttgcgtcaacggggcggggttattacgacattttggaaagtcccgttgattttggtgccaaaacaaactcccattgacgtcaatggggtggagacttggaaatccccgtgagtcaaaccgctatccacgcccattggtgtactgccaaaaccgcatcaccatggtaatagcgatgactaatacgtagatgtactgccaagtaggaaagtcccgtaaggtcatgtactgggcataatgccaggcgggccatttaccgtcattgacgtcaatagggggcggacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcggaactccatatatgggctatgaactaatgaacccgtaattgattactattaataactagtcaataatcaatgtcaacatggcggtcatattggacatgagccaatataaatgtacatattatgatatagatacaacgtatgcaatggccaatagccaatattgtcg

SEQ ID NO: 38 - DNA, 2947 bp, Artificial Sequence
  Vaccine vector pGA2
acaacatgtgagcaaaaggccagcaaaaggccaggaaccgtaaaagggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttcacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcagaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaatttgtcgactctagcgttcagaacgctcggttgccgccgggcgttttttatatagagcccaccgcatccccagcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagcctagggattgcgcggtccgtttatcacccggggctagccgaaacgaagactgctccacacagcagcagcacacagcagagccctctcttcattgcatccatgattgcaagcatcgatagaatgagttcactaaacgagctctgcttatatagacctcccaccgtacacgcctaccgcccatttgcgtcaacggggcggggttattacgacattttggaaagtcccgttgattttggtgccaaaacaaactcccattgacgtcaatggggtggagacttggaaatccccgtgagtcaaaccgctatccacgcccattggtgtactgccaaaaccgcatcaccatggtaatagcgatgactaatacgtagatgtactgccaagtaggaaagtcccgtaaggtcatgtactgggcataatgccaggcgggccatttaccgtcattgacgtcaatagggggcggacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcggaactccatatatgggctatgaactaatgaccccgtaattgattactattaataactagtcaataatcaatgtcaacatggcggtcatattggacatgagccaatataaatgtacatattatgatatagatacaacgtatgcaatggccaatagccaatattgtcg

SEQ ID NO: 39 - DNA, 3893 bp, Artificial Sequence
  Vaccine vector pGA3
acaacatgtgagcaaaaggccagcaaaaggccaggaaccgtaaaagggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttcacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcagaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaatttgtcgactctagcgttcagaacgctcggttgccgccgggcgttttttatatagagcccaccgcatccccagcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagcctagggattgcgaggatccttatcacccggggctagccgaaacgaagactgctccacacagcagcagcacacagcagagccctctcttcattgcatccatgattgcaagcttggacggtgactgcagaaaagacccatggaaaggaacagtctgttagtctgtcagctattatgtctggtggcgcgcgcggcagcaacgagtactgctcagactacactgccctccaccgttaacagcaccgcaacgggagttacctctgactcttatcagaatacaacaactcagctgcctgcatcttcttctgccgctgccttaagtcttccatctgcgtcagcggtgcgagcccaatctccgagctcattttcagacacataccctaccgccacggccttgtgcggcacactggtggtggtgggcattgtgctgtgcctaagtctggcctccactgttaggagcaaggagctgccgagcgaccatgagccgctggaggcatgggaccagggctcggatgtggaagctccgccgctaccggagaagagcccatgtccggaacacgtacccgagattcgcgtggagatcccacgctatgtttaataaaaactgcgggcacgggggacggcgttgttgtatatgtgaatttgtaaataataaatgggaccccatcctgtaaaaatacagagtccgtgtcagtctctgaaggacagagtattggcatatagccaatagagatagttgtggcaaagagccatgttatggattagtaatggaaagtatcgtcaccaataggggagtggtcaataatggtcaataacccacacctataggctaagctataccatcacctatagcataaggaagcgggggtgtataggccccaagccaaaaacagtatagcatgcataagagccaaaggggtgtgcctatagagtctataggcggtacttacgtcactcttggcacggggaatccgcgttccaatgcaccgttcccggccgcggaggctggatcggtcccggtgtcttctatggaggtcaaaacagcgtggatggcgtctccaggcgatctgacggttcactaaacgagctctgcttatatagacctcccaccgtacacgcctaccgcccatttgcgtcaacggggcggggttattacgacattttggaaagtcccgttgattttggtgccaaaacaaactcccattgacgtcaatggggtggagacttggaaatccccgtgagtcaaaccgctatccacgcccattggtgtactgccaaaaccgcatcaccatggtaatagcgatgactaatacgtagatgtactgccaagtaggaaagtcccgtaaggtcatgtactgggcataatgccaggcgggccatttaccgtcattgacgtcaatagggggcggacttggcatatgatacacttgatgtactgccaagtgggcagtttaccgtaaatactccacccattgacgtcaatggaaagtccctattggcgttactatgggaacatacgtcattattgacgtcaatgggcgggggtcgttggggggtcagccaggcgggccatttaccgtaagttatgtaacgcggaactccatatatgggctatgaactaatgaccccgtaattgattactattaataactagtcaataatcaatgtcaacatggcggtcatattggacatgagccaatataaatgtacatattatgatatagatacaacgtatgcaatggccaatagccaatattgtcg

SEQ ID NO: 40 - DNA, 3086 bp, Artificial Sequence
  Synthetically generated oligonucleotide
aaggggttaaagctataataagaattctgcaacagctactgtttgttcatttcagaattgggtgtcaacatagcagaataggcattattccagggagaagaggcaggaatggagctggtagatcctagcctagagccctggaaccacccgggaagtcagcctacaactgcttgtagcaagtgttactgtaaaaaatgctgctggcattgccaattgtgctttctgaacaagggcttaggcatctcctatggcaggaagaagcggagacgccgacgaggaactcctcaggaccgtcaggttcatcaaaatcctgtaccaaaacagtaagtagtagtaattagtatatgtgatgcaatctttacaaatagctgcaatagtaggactagtagtagcatccatagtagccatagttgtgtggtccatagtatttatagaatatagaaaaataaggaaacagaagaaaatagacaggttacttgagagaataagagaaagagcagaagatagtggcaatgagagtgatggggatacagaagaattatccactcttatggagagggggtatgacaatattttggttaatgatgatttgtaatgctgaaaagttgtgggtcacagtctactatggggtacctgtgtggagagacgcagagaccaccctattctgtgcatcagatgctaaagcatatgacaaagaagcacacaatgtctgggctacgcatgcctgcgtacccacagaccctgacccacaagaattacctttggtaaatgtaacagaagagtttaacatgtggaaaaataatatggtagaacagatgcatgaagatataattagtctatgggaccaaagcttaaagccatgtgtacagctaacccctctctgcgttactttagggtgtgctgacgctcaaaacgtcaccgacaccaacaccaccatatctaatgaaatgcaaggggaaataaaaaactgctctttcaatatgaccacagaattaagagataagaagcagaaagtgtatgcacttttttatagacctgatgtaatagaaattaataaaactaagattaacaatagtaatagtagtcagtatatgttaataaattgtaatacctcaaccattacacagacttgtccaaaggtatcctttgagccaattcccatacattattgtgccccagctggttttgcaattctaaagtgtaatgatacggagttcagtggaaaagggacatgcaagagtgtcagcacagtacaatgcacacatggaatcaagccagtagtatcaactcaactgctgttaaatggcagtctagcagaaggaaagatagcgattagatctgagaatatctcaaacaatgccaaaactataatagtacaattgactgagcctgtagaaattaattgtatcagacctggcaacaatacaagaaaaagtgtacgcataggaccaggacaaacattctatgcaacaggtgacataataggagatataagacaagcacactgtaatgttagtaaaatagcatgggaagaaactttacaaaaggtagctgcacaattaaggaagcactttcagaatgccacaataaaatttactaaacactcaggaggggatttagaaattacaacaaatagttttaattgtggaggagaatttttctattgcaatacaacaaagctgtttaatagcacttggaataatgataactcaaacctcacagaggaaaagagaaaggaaaacataactctccactgcagaataaagcaaattgtaaatatgtggccaagagtaggncaagcaatatatgcccctcccatcccaggaaacataacttgtggatcaaacattactgggctactattaacaagagatggagggaataatggtacaaatgatactgagaccttcaggcctggaggaggagatatgagggacaattggagaagtgaattatataaatataaagtagtaaaaattgaaccactaggtgtagcaccaacccctgcaaaaagaagagtggtggaaagagaaaaaagagcagttggaatgggagctttgatctttgagttcttaggagcagcaggaagcactatgggcgcggcgtcaatggcgctgacggtacaggccagacaattattgtctggtatagtgcaacagcagagcaatctgctgaaggctatagaggctcaacaacatctgttgagactcacggtctggggcattaaacagctccaggcaagagtcctggctctggaaagatacctaaaggatcaacagctcctaggaatttggggctgctctggaaaactcatttgcaccactgctgtaccttggaactctagctggagtaataaaagttataatgacatatgggataacatgacctggctgcaatgggataaagaaattaacaattacacatacataatatataatctacttgaaaaatcgcagaaccagcaggaaattaatgaacaagacttattggcattagacaagtgggcaagtctgtggaattggtttgacataacaagctggctatggtatataagattaggtataatgatagtaggaggcgtaataggcttaagaataatttttgctgtgcttactatagtgaatagagttaggcagggatactcacctttgtcattccagacccttgcccaccaccagagggaacccgacaggcccgaaagaatcgaagaaggaggtggcgagcaagacagagagagatccgtgcgcttagtgagcggattcttagcacttgcctgggaagatctgcggagcctgtgcctcttcagctaccgccgattgagagacttagtcttgattgcagcaaggactgtggaactcctgggacacagcagtctcaagggactgagactggggtgggaagccctcaaatatctgtggaaccttctatcatactggggtcaggaactaaagaatagtgctattaatttgcttgatacaatagcaatagcagtagctaactggacagatagagttataaaaatagtacaaagaactggtagagctattcttaacatacctagaaggatcagatagggctagcaaagg

SEQ ID NO: 41 - DNA, 3575 bp, Artificial Sequence
  Synthetically generated oligonucleotide
gcaaggactcggcttgctgaggtgcacacagcaagaggcgagagcgacgactggtgagtacgccaatttttgactagcggaggctagaaggagagagatgggtgcgagagcgtcagtgttaacggggggaaaattagattcatgggagaaaaataggttaaggccagggggaaagaaaagatatagactaaaacacctagtatgggcaagcagggagctggagagattcgcacttaaccctggcctattagaaacagcagaaggatgtcaacaactaatggaacagttacaaccagctctcaggacaggatcagaagagtttaaatcattacataatacagtagcaaccctttggtgcgtacatcaaagaatagacataaaagacacccaggaggccttagataaagtagaggaaaaacaaaataagagcaagcaaaaggcacagcaggcagcagctgcaacagccgccacaggaagcagcagccaaaattaccctatagtgcaaaatgcacaagggcaaatggtacatcagtccatgtcacctaggactttaaatgcatgggtgaaggtaatagaagaaaaggcttttagcccagaggtaatacccatgttttcagcattatcagagggagccaccccacaagatttaaatatgatgctaaacatagtggggggacaccaggcagcaatgcagatgttaaaagataccatcaatgatgaagctgcagaatgggacagagtacatccagtacatgcagggcctattccaccaggccaaatgagggaaccaaggggaagtgacatagcaggaactactagtacccttcaagaacaaataggatggatgacaagtaatccacctatcccagtgggagaaatctataaaagatggatagtcctgggattaaataaaatagtaagaatgtatagccctaccagcattttggacataagacaagggccaaaagaaccctttagagattatgtagacaggttctttaaaactttgagagctgaacaagctacgcaggaggtaaaaaactggatgacagaaaccttgttggtccaaaatgcgaatccagactgcaagtccattttaagagcaataggaccaggggctacattagaagaaatgatgacatcatgtcagggagtgggaggacctggccataaagcaagggttttggctggggcaatgagtcaagtacaacagaccaatgtaatgatgcagagaggcaattttagaggccagagaataataaagtgtttcaactgtggcaaagaaggacacctagccagaaattgcaaggctcctagaaagagaggctgttggaaatgtggaaaggaaggacaccaaatgaaagactgtactgaaaaacaggctaattttttagggaaaatttggccttcccacaaggggaggccaggaaattttcctcagagcagaccagaaccaacagccccgccagcagagagctttggagtgggggaagagataccctcctctccgaagcaggagccgagggacaagggactatatcctcccttaacttccctcaaatcactctttggcaacgaccagtagtcacagtaagaatagggggacagccaatagaagccctattagacacaggagcagatgatacagtattagaagaaataagtttaccaggaaaatggaaaccaaaaatgatagggggaattggaggttttatcaaagtaagacagtatgatcagatatctatagaaatttgtggaaaaagggccataggtacagtattagtaggacctacacctgtcaacataattggacgaaatatgttgactcagattggttgtactttaaattttccaattagtcctattgaaactgtgtcagtaaaattaaagccaggaatggatggcccaaaggttaaacaatggccattgacagaagaaaaaataaaagcattaaaagaaatttgtgcagagatggaaaaggaaggaaaaatttcaaaaattgggcctgaaaacccatacaatactccaatatttgccataaagaaaaaagatagtactaaatggagaaaattagtagatttcagagaactcaataagagaactcaagacttctgggaggtccaattaggaatacctcatcctgcgggattaaaaaagaaaaaatcagtaacagtactagatgtgggggatgcatatttttcagttcccgtagatgaagactttagaaaatatactgcattcaccatacctagtttaaataatgagacaccagggattagatatcagtacaatgtactcccacagggatggaaaggatcaccagcaatatttcaggcaagcatgacaaaaatcttagagccctttagagcaaaaaatccagagatagtgatctaccaatatatggatgatttatatgtaggatctgacttagaaatagggcagcatagagcaaaaatagaggagttgagagaacatctattgaaatggggatttaccacaccagacaaaaaacatcagaaagaacctccatttctttggatgggatatgaactccatcctgacaaatggacagtccagcctatacagctgccagaaaaagacagctggactgtcaatgatatacaaaaattagtgggaaaactaaattgggcaagtcagatttatgcaggaattaaagtaaagcaattgtgtagactcctcaggggagccaaagcgctaacagatgtagtaacactgactgaggaagcagaattagaattggcagagaacagggaaattctaaaagaacctgtacatggagtatattatgacccaacaaaagacttagtggcagaaatacagaaacaagggcaagatcaatggacatatcaaatttatcaagagccatttaaaaatctaaagacaggaaaatatgcaaaaaagaggtcggcccacactaatgatgtaaaacaattaacagaggtagtgcagaaaatagccatagaaagcatagtaatatggggaaagacccctaaatttagactacccatacaaagagaaacatgggaagcatggtggatggagtattggcaggctacctggattcctgaatgggagtttgtcaatacccctcctctagtaaaattatggtaccagttagagaaggaccccataatgggagcagaaactttctatgtagatggggcagctaatagggagactaagctaggaaaagcagggtatgtcactgacagaggaagacaaaaggttgtttccctaattgagacaacaaatcaaaagactgaattacatgcaattcatctagccttgcaggattcaggatcagaagtaaatatagtaacagactcacagtatgcattaggaatcattcaggcacaaccagacaggagtgaatcagagttagtcaatcaaataatagagaaactaatagaaaaggacaaagtctacctgtcatgggtaccagcacacaaagggattggaggaaatgaacaagtagataaattagtcagtagtggaatcagaaaggtactatttttagatggaatagataaagcccaagatgaacattagaattc

SEQ ID NO: 42 - DNA, 3575 bp, Artificial Sequence
  Synthetically generated oligonucleotide
gcaaggactcggcttgctgaggtgcacacagcaagaggcgagagcgacgactggtgagtacgccaatttttgactagcggaggctagaaggagagagatgggtgcgagagcgtcagtgttaacggggggaaaattagattcatgggagaaaaataggttaaggccagggggaaagaaaagatatagactaaaacacctagtatgggcaagcagggagctggagagattcgcacttaaccctggcctattagaaacagcagaaggatgtcaacaactaatggaacagttacaaccagctctcaggacaggatcagaagagtttaaatcattacataatacagtagcaaccctttggtgcgtacatcaaagaatagacataaaagacacccaggaggccttagataaagtagaggaaaaacaaaataagagcaagcaaaaggcacagcaggcagcagctgcaacagccgccacaggaagcagcagccaaaattaccctatagtgcaaaatgcacaagggcaaatggtacatcagtccatgtcacctaggactttaaatgcatgggtgaaggtaatagaagaaaaggcttttagcccagaggtaatacccatgttttcagcattatcagagggagccaccccacaagatttaaatatgatgctaaacatagtggggggacaccaggcagcaatgcagatgttaaaagataccatcaatgatgaagctgcagaatgggacagagtacatccagtacatgcagggcctattccaccaggccaaatgagggaaccaaggggaagtgacatagcaggaactactagtacccttcaagaacaaataggatggatgacaagtaatccacctatcccagtgggagaaatctataaaagatggatagtcctgggattaaataaaatagtaagaatgtatagccctaccagcattttggacataagacaagggccaaaagaaccctttagagattatgtagacaggttctttaaaactttgagagctgaacaagctacgcaggaggtaaaaaactggatgacagaaaccttgttggtccaaaatgcgaatccagactgcaagtccattttaagagcaataggaccaggggctacattagaagaaatgatgacatcatgtcagggagtgggaggacctggccataaagcaagggttttggctggggcaatgagtcaagtacaacagaccaatgtaatgatgcagagaggcaattttagaggccagagaataataaagagtttcaacagtggcaaagaaggacacctagccagaaattgcaaggctcctagaaagagaggcagttggaaaagtggaaaggaaggacaccaaatgaaagactgtactgaaaaacaggctaattttttagggaaaatttggccttcccacaaggggaggccaggaaattttcctcagagcagaccagaaccaacagccccgccagcagagagctttggagtgggggaagagataccctcctctccgaagcaggagccgagggacaagggactatatcctcccttaacttccctcaaatcactctttggcaacgaccagtagtcacagtaagaatagggggacagccaatagaagccctattagacacaggagcagatgatacagtattagaagaaataagtttaccaggaaaatggaaaccaaaaatgatagggggaattggaggttttatcaaagtaagacagtatgatcagatatctatagaaatttgtggaaaaggggccataggtacagtattagtaggacctacacctgtcaacataattggacgaaatatgttgactcagattggttgtactttaaattttccaattagtcctattgaaactgtgtcagtaaaattaaagccaggaatggatggcccaaaggttaaacaatggccattgacagaagaaaaaataaaagcattaaaagaaatttgtgcagagatggaaaaggaaggaaaaatttcaaaaattgggcctgaaaacccatacaatactccaatatttgccataaagaaaaaagatagtactaaatggagaaaattagtagatttcagagaactcaataagagaactcaagacttctgggaggtccaattaggaatacctcatcctgcgggattaaaaaagaaaaaatcagtaacagtactagatgtgggggatgcatatttttcagttcccgtagatgaagactttagaaaatatactgcattcaccatacctagtttaaataatgagacaccagggattagatatcagtacaatgtactcccacagggatggaaaggatcaccagcaatatttcaggcaagcatgacaaaaatcttagagccctttagagcaaaaaatccagagatagtgatctaccaatatatgaatgatttatatgtaggatctgacttagaaatagggcagcatagagcaaaaatagaggagttgagagaacatctattgaaatggggatttaccacaccagacaaaaaacatcagaaagaacctccatttctttggatgggatatgaactccatcctgacaaatggacagtccagcctatacagctgccagaaaaagacagctggactgtcaatgatatacaaaaattagtgggaaaactaaatacggcaagtcagatttatgcaggaattaaagtaaagcaattgtgtagactcctcaggggagccaaagcgctaacagatgtagtaacactgactgaggaagcagaattagaattggcagagaacagggaaattctaaaagaacctgtacatggagtatattatgacccaacaaaagacttagtggcagaaatacagaaacaagggcaagatcaatggacatatcaaatttatcaagagccatttaaaaatctaaagacaggaaaatatgcaaaaaagaggtcggcccacactaatgatgtaaaacaattaacagaggtagtgcagaaaatagccatagaaagcatagtaatatggggaaagacccctaaatttagactacccatacaaagagaaacatgggaagcatggtggatggagtattggcaggctacctggattcctgaatgggagtttgtcaatacccctcctctagtaaaattatggtaccagttagagaaggaccccataatgggagcagaaactttctatgtagatggggcagctaatagggagactaagctaggaaaagcagggtatgtcactgacagaggaagacaaaaggttgtttccctaattgagacaacaaatcaaaagactcaattacatgcaattcatctagccttgcaggattcaggatcagaagtaaatatagtaacagactcacagtatgcattaggaatcattcaggcacaaccagacaggagtgaatcagagttagtcaatcaaataatagagaaactaatagaaaaggacaaagtctacctgtcatgggtaccagcacacaaagggattggaggaaatgaacaagtagataaattagtcagtagtggaatcagaaaggtactatttttagatggaatagataaagcccaagatgaacattagaattc

SEQ ID NO: 43 - DNA, 3575 bp, Artificial Sequence
  Synthetically generated oligonucleotide
gcaaggactcggcttgctgaggtgcacacagcaagaggcgagagcgacgactggtgagtacgccaatttttgactagcggaggctagaaggagagagatgggtgcgagagcgtcagtgttaacggggggaaaattagattcatgggagaaaaataggttaaggccagggggaaagaaaagatatagactaaaacacctagtatgggcaagcagggagctggagagattcgcacttaaccctggcctattagaaacagcagaaggatgtcaacaactaatggaacagttacaaccagctctcaggacaggatcagaagagtttaaatcattacataatacagtagcaaccctttggtgcgtacatcaaagaatagacataaaagacacccaggaggccttagataaagtagaggaaaaacaaaataagagcaagcaaaaggcacagcaggcagcagctgcaacagccgccacaggaagcagcagccaaaattaccctatagtgcaaaatgcacaagggcaaatggtacatcagtccatgtcacctaggactttaaatgcatgggtgaaggtaatagaagaaaaggcttttagcccagaggtaatacccatgttttcagcattatcagagggagccaccccacaagatttaaatatgatgctaaacatagtggggggacaccaggcagcaatgcagatgttaaaagataccatcaatgatgaagctgcagaatgggacagagtacatccagtacatgcagggcctattccaccaggccaaatgagggaaccaaggggaagtgacatagcaggaactactagtacccttcaagaacaaataggatggatgacaagtaatccacctatcccagtgggagaaatctataaaagatggatagtcctgggattaaataaaatagtaagaatgtatagccctaccagcattttggacataagacaagggccaaaagaaccctttagagattatgtagacaggttctttaaaactttgagagctgaacaagctacgcaggaggtaaaaaactggatgacagaaaccttgttggtccaaaatgcgaatccagactgcaagtccattttaagagcaataggaccaggggctacattagaagaaatgatgacatcatgtcagggagtgggaggacctggccataaagcaagggttttggctggggcaatgagtcaagtacaacagaccaatgtaatgatgcagagaggcaattttagaggccagagaataataaagagtttcaacagtggcaaagaaggacacctagccagaaattgcaaggctcctagaaagagaggcagttggaaaagtggaaaggaaggacaccaaatgaaagactgtactgaaaaacaggctaattttttagggaaaatttggccttcccacaaggggaggccaggaaattttcctcagagcagaccagaaccaacagccccgccagcagagagctttggagtgggggaagagataccctcctctccgaagcaggagccgagggacaagggactatatcctcccttaacttccctcaaatcactctttggcaacgaccagtagtcacagtaagaatagggggacagccaatagaagccctattagccacaggagcagatgatacagtattagaagaaataagtttaccaggaaaatggaaaccaaaaatgatagggggaattggaggttttatcaaagtaagacagtatgatcagatatctatagaaatttgtggaaaaggggccataggtacagtattagtaggacctacacctgtcaacataattggacgaaatatgttgactcagattggttgtactttaaattttccaattagtcctattgaaactgtgtcagtaaaattaaagccaggaatggatggcccaaaggttaaacaatggccattgacagaagaaaaaataaaagcattaaaagaaatttgtgcagagatggaaaaggaaggaaaaatttcaaaaattgggcctgaaaacccatacaatactccaatatttgccataaagaaaaaagatagtactaaatggagaaaattagtagatttcagagaactcaataagagaactcaagacttctgggaggtccaattaggaatacctcatcctgcgggattaaaaaagaaaaaatcagtaacagtactagatgtgggggatgcatatttttcagttcccgtagatgaagactttagaaaatatactgcattcaccatacctagtttaaataatgagacaccagggattagatatcagtacaatgtactcccacagggatggaaaggatcaccagcaatatttcaggcaagcatgacaaaaatcttagagccctttagagcaaaaaatccagagatagtgatctaccaatatatgaatgatttatatgtaggatctgacttagaaatagggcagcatagagcaaaaatagaggagttgagagaacatctattgaaatggggatttaccacaccagacaaaaaacatcagaaagaacctccatttctttggatgggatatgaactccatcctgacaaatggacagtccagcctatacagctgccagaaaaagacagctggactgtcaatgatatacaaaaattagtgggaaaactaaatacggcaagtcagatttatgcaggaattaaagtaaagcaattgtgtagactcctcaggggagccaaagcgctaacagatgtagtaacactgactgaggaagcagaattagaattggcagagaacagggaaattctaaaagaacctgtacatggagtatattatgacccaacaaaagacttagtggcagaaatacagaaacaagggcaagatcaatggacatatcaaatttatcaagagccatttaaaaatctaaagacaggaaaatatgcaaaaaagaggtcggcccacactaatgatgtaaaacaattaacagaggtagtgcagaaaatagccatagaaagcatagtaatatggggaaagacccctaaatttagactacccatacaaagagaaacatgggaagcatggtggatggagtattggcaggctacctggattcctgaatgggagtttgtcaatacccctcctctagtaaaattatggtaccagttagagaaggaccccataatgggagcagaaactttctatgtagatggggcagctaatagggagactaagctaggaaaagcagggtatgtcactgacagaggaagacaaaaggttgtttccctaattgagacaacaaatcaaaagactcaattacatgcaattcatctagccttgcaggattcaggatcagaagtaaatatagtaacagactcacagtatgcattaggaatcattcaggcacaaccagacaggagtgaatcagagttagtcaatcaaataatagagaaactaatagaaaaggacaaagtctacctgtcatgggtaccagcacacaaagggattggaggaaatgaacaagtagataaattagtcagtagtggaatcagaaaggtactatttttagatggaatagataaagcccaagatgaacattagaattc

SEQ ID NO: 44 - DNA, 3575 bp, Artificial Sequence
  Synthetically generated oligonucleotide
gcaaggactcggcttgctgaggtgcacacagcaagaggcgagagcgacgactggtgagtacgccaatttttgactagcggaggctagaaggagagagatgggtgcgagagcgtcagtgttaacggggggaaaattagattcatgggagaaaaataggttaaggccagggggaaagaaaagatatagactaaaacacctagtatgggcaagcagggagctggagagattcgcacttaaccctggcctattagaaacagcagaaggatgtcaacaactaatggaacagttacaaccagctctcaggacaggatcagaagagtttaaatcattacataatacagtagcaaccctttggtgcgtacatcaaagaatagacataaaagacacccaggaggccttagataaagtagaggaaaaacaaaataagagcaagcaaaaggcacagcaggcagcagctgcaacagccgccacaggaagcagcagccaaaattaccctatagtgcaaaatgcacaagggcaaatggtacatcagtccatgtcacctaggactttaaatgcatgggtgaaggtaatagaagaaaaggcttttagcccagaggtaatacccatgttttcagcattatcagagggagccaccccacaagatttaaatatgatgctaaacatagtggggggacaccaggcagcaatgcagatgttaaaagataccatcaatgatgaagctgcagaatgggacagagtacatccagtacatgcagggcctattccaccaggccaaatgagggaaccaaggggaagtgacatagcaggaactactagtacccttcaagaacaaataggatggatgacaagtaatccacctatcccagtgggagaaatctataaaagatggatagtcctgggattaaataaaatagtaagaatgtatagccctaccagcattttggacataagacaagggccaaaagaaccctttagagattatgtagacaggttctttaaaactttgagagctgaacaagctacgcaggaggtaaaaaactggatgacagaaaccttgttggtccaaaatgcgaatccagactgcaagtccattttaagagcaataggaccaggggctacattagaagaaatgatgacatcatgtcagggagtgggaggacctggccataaagcaagggttttggctggggcaatgagtcaagtacaacagaccaatgtaatgatgcagagaggcaattttagaggccagagaataataaagagtttcaacagtggcaaagaaggacacctagccagaaattgcaaggctcctagaaagagaggcagttggaaaagtggaaaggaaggacaccaaatgaaagactgtactgaaaaacaggctaattttttagggaaaatttggccttcccacaaggggaggccaggaaattttcctcagagcagaccagaaccaacagccccgccagcagagagctttggagtgggggaagagataccctcctctccgaagcaggagccgagggacaagggactatatcctcccttaacttccctcaaatcactctttggcaacgaccagtagtcacagtaagaatagggggacagccaatagaagccctattagacacaggagcagatgatacagtattagaagaaataagtttaccaggaaaatggaaaccaaaaatgatagtgggaattggaggttttatcaaagtaagacagtatgatcagatatctatagaaatttgtggaaaaggggccataggtacagtattagtaggacctacacctgtcaacataattggacgaaatatgttgactcagattggttgtactttaaattttccaattagtcctattgaaactgtgtcagtaaaattaaagccaggaatggatggcccaaaggttaaacaatggccattgacagaagaaaaaataaaagcattaaaagaaatttgtgcagagatggaaaaggaaggaaaaatttcaaaaattgggcctgaaaacccatacaatactccaatatttgccataaagaaaaaagatagtactaaatggagaaaattagtagatttcagagaactcaataagagaactcaagacttctgggaggtccaattaggaatacctcatcctgcgggattaaaaaagaaaaaatcagtaacagtactagatgtgggggatgcatatttttcagttcccgtagatgaagactttagaaaatatactgcattcaccatacctagtttaaataatgagacaccagggattagatatcagtacaatgtactcccacagggatggaaaggatcaccagcaatatttcaggcaagcatgacaaaaatcttagagccctttagagcaaaaaatccagagatagtgatctaccaatatatgaatgatttatatgtaggatctgacttagaaatagggcagcatagagcaaaaatagaggagttgagagaacatctattgaaatggggatttaccacaccagacaaaaaacatcagaaagaacctccatttctttggatgggatatgaactccatcctgacaaatggacagtccagcctatacagctgccagaaaaagacagctggactgtcaatgatatacaaaaattagtgggaaaactaaatacggcaagtcagatttatgcaggaattaaagtaaagcaattgtgtagactcctcaggggagccaaagcgctaacagatgtagtaacactgactgaggaagcagaattagaattggcagagaacagggaaattctaaaagaacctgtacatggagtatattatgacccaacaaaagacttagtggcagaaatacagaaacaagggcaagatcaatggacatatcaaatttatcaagagccatttaaaaatctaaagacaggaaaatatgcaaaaaagaggtcggcccacactaatgatgtaaaacaattaacagaggtagtgcagaaaatagccatagaaagcatagtaatatggggaaagacccctaaatttagactacccatacaaagagaaacatgggaagcatggtggatggagtattggcaggctacctggattcctgaatgggagtttgtcaatacccctcctctagtaaaattatggtaccagttagagaaggaccccataatgggagcagaaactttctatgtagatggggcagctaatagggagactaagctaggaaaagcagggtatgtcactgacagaggaagacaaaaggttgtttccctaattgagacaacaaatcaaaagactcaattacatgcaattcatctagccttgcaggattcaggatcagaagtaaatatagtaacagactcacagtatgcattaggaatcattcaggcacaaccagacaggagtgaatcagagttagtcaatcaaataatagagaaactaatagaaaaggacaaagtctacctgtcatgggtaccagcacacaaagggattggaggaaatgaacaagtagataaattagtcagtagtggaatcagaaaggtactatttttagatggaatagataaagcccaagatgaacattagaattc

SEQ ID NO: 45 - DNA, 3575 bp, Artificial Sequence
  Synthetically generated oligonucleotide
gcaaggactcggcttgctgaggtgcacacagcaagaggcgagagcgacgactggtgagtacgccaatttttgactagcggaggctagaaggagagagatgggtgcgagagcgtcagtgttaacggggggaaaattagattcatgggagaaaaataggttaaggccagggggaaagaaaagatatagactaaaacacctagtatgggcaagcagggagctggagagattcgcacttaaccctggcctattagaaacagcagaaggatgtcaacaactaatggaacagttacaaccagctctcaggacaggatcagaagagtttaaatcattacataatacagtagcaaccctttggtgcgtacatcaaagaatagacataaaagacacccaggaggccttagataaagtagaggaaaaacaaaataagagcaagcaaaaggcacagcaggcagcagctgcaacagccgccacaggaagcagcagccaaaattaccctatagtgcaaaatgcacaagggcaaatggtacatcagtccatgtcacctaggactttaaatgcatgggtgaaggtaatagaagaaaaggcttttagcccagaggtaatacccatgttttcagcattatcagagggagccaccccacaagatttaaatatgatgctaaacatagtggggggacaccaggcagcaatgcagatgttaaaagataccatcaatgatgaagctgcagaatgggacagagtacatccagtacatgcagggcctattccaccaggccaaatgagggaaccaaggggaagtgacatagcaggaactactagtacccttcaagaacaaataggatggatgacaagtaatccacctatcccagtgggagaaatctataaaagatggatagtcctgggattaaataaaatagtaagaatgtatagccctaccagcattttggacataagacaagggccaaaagaaccctttagagattatgtagacaggttctttaaaactttgagagctgaacaagctacgcaggaggtaaaaaactggatgacagaaaccttgttggtccaaaatgcgaatccagactgcaagtccattttaagagcaataggaccaggggctacattagaagaaatgatgacatcatgtcagggagtgggaggacctggccataaagcaagggttttggctggggcaatgagtcaagtacaacagaccaatgtaatgatgcagagaggcaattttagaggccagagaataataaagagtttcaacagtggcaaagaaggacacctagccagaaattgcaaggctcctagaaagagaggcagttggaaaagtggaaaggaaggacaccaaatgaaagactgtactgaaaaacaggctaattttttagggaaaatttggccttcccacaaggggaggccaggaaattttcctcagagcagaccagaaccaacagccccgccagcagagagctttggagtgggggaagagataccctcctctccgaagcaggagccgagggacaagggactatatcctcccttaacttccctcaaatcactctttggcaacgaccagtagtcacagtaagaatagggggacagccaatagaagccctattagacacaggagcagatgatacagtattagaagaaataagtttaccaggaaaatggaaaccaaaaatgatagggggaattggaggttttatcaaagtaagacagtatgatcagatatctatagaaatttgtggaaaaggggccataggtacagtattagtaggacctacacctgtcaacataattggacgaaatatgatgactcagattggttgtactttaaattttccaattagtcctattgaaactgtgtcagtaaaattaaagccaggaatggatggcccaaaggttaaacaatggccattgacagaagaaaaaataaaagcattaaaagaaatttgtgcagagatggaaaaggaaggaaaaatttcaaaaattgggcctgaaaacccatacaatactccaatatttgccataaagaaaaaagatagtactaaatggagaaaattagtagatttcagagaactcaataagagaactcaagacttctgggaggtccaattaggaatacctcatcctgcgggattaaaaaagaaaaaatcagtaacagtactagatgtgggggatgcatatttttcagttcccgtagatgaagactttagaaaatatactgcattcaccatacctagtttaaataatgagacaccagggattagatatcagtacaatgtactcccacagggatggaaaggatcaccagcaatatttcaggcaagcatgacaaaaatcttagagccctttagagcaaaaaatccagagatagtgatctaccaatatatgaatgatttatatgtaggatctgacttagaaatagggcagcatagagcaaaaatagaggagttgagagaacatctattgaaatggggatttaccacaccagacaaaaaacatcagaaagaacctccatttctttggatgggatatgaactccatcctgacaaatggacagtccagcctatacagctgccagaaaaagacagctggactgtcaatgatatacaaaaattagtgggaaaactaaatacggcaagtcagatttatgcaggaattaaagtaaagcaattgtgtagactcctcaggggagccaaagcgctaacagatgtagtaacactgactgaggaagcagaattagaattggcagagaacagggaaattctaaaagaacctgtacatggagtatattatgacccaacaaaagacttagtggcagaaatacagaaacaagggcaagatcaatggacatatcaaatttatcaagagccatttaaaaatctaaagacaggaaaatatgcaaaaaagaggtcggcccacactaatgatgtaaaacaattaacagaggtagtgcagaaaatagccatagaaagcatagtaatatggggaaagacccctaaatttagactacccatacaaagagaaacatgggaagcatggtggatggagtattggcaggctacctggattcctgaatgggagtttgtcaatacccctcctctagtaaaattatggtaccagttagagaaggaccccataatgggagcagaaactttctatgtagatggggcagctaatagggagactaagctaggaaaagcagggtatgtcactgacagaggaagacaaaaggttgtttccctaattgagacaacaaatcaaaagactcaattacatgcaattcatctagccttgcaggattcaggatcagaagtaaatatagtaacagactcacagtatgcattaggaatcattcaggcacaaccagacaggagtgaatcagagttagtcaatcaaataatagagaaactaatagaaaaggacaaagtctacctgtcatgggtaccagcacacaaagggattggaggaaatgaacaagtagataaattagtcagtagtggaatcagaaaggtactatttttagatggaatagataaagcccaagatgaacattagaattc

SEQ ID NO: 46 - protein, 29 aa, Artificial Sequence
  Synthetically generated peptide
PTSQPRGDPTGPKESKKKVETETETDPCD

SEQ ID NO: 47 - protein, 91 aa, Artificial Sequence
  Synthetically generated peptide
NPPPSPEGTRQARRNRRRRWRQRQRQIRAISGWILSTYLGRSAEPVPLQLPPLERLTLDCNEDCGTSGTQGVGSPQILVESPTVLESGAKE

SEQ ID NO: 48 - protein, 14 aa, Artificial Sequence
  Synthetically generated peptide
PTSQSRGDPTGPKE

SEQ ID NO: 49 - protein, 91 aa, Artificial Sequence
  Synthetically generated peptide
NPPPNPEGTRQARRNRRRRWRERQRQIHSISERILSTYLGRSAEPVPLQLPPLERLTLDCNEDCGTSGTQGVGSPQILVESPTVLESGAKE

SEQ ID NO: 50 - protein, 9 aa, Artificial Sequence
  synthetically generated peptide
CTPYDINQM

SEQ ID NO: 51 - protein, 6 aa, HIV-1
VAPTRA

SEQ ID NO: 52 - protein, 18 aa, Artificial Sequence
  tpa leader sequence of pGA3
MKRGLCCVLLLCGAVFVS